SEQUENCE LISTING (71 sequences)

SEQ ID NO: 1 - protein, 136 aa, Artificial Sequence
  CA12760 GFP-stripper amino acid sequence
QVQLVESGGGLVQAGGSLRLSCAASGRTFSTAAMGWFRQAPGKERDFVAGIYWTVGSTYYADSAKGRFTISRDNAKNTVYLQMDSLKPEDTAVYYCAARRRGFTLAPTRANEYDYWGQGTQVTVSSHHHHHHEPEA

SEQ ID NO: 2 - protein, 136 aa, Artificial Sequence
  CA15816 T54A/V55A mutant GFP-trapper amino acid sequence
QVQLVESGGGLVQAGGSLRLSCAASGRTFSTAAMGWFRQAPGKERDFVAGIYWAAGSTYYADSAKGRFTISRDNAKNTVYLQMDSLKPEDTAVYYCAARRRGFTLAPTRANEYDYWGQGTQVTVSSHHHHHHEPEA

SEQ ID NO: 3 - protein, 129 aa, Artificial Sequence
  CA17440 FBA1 binder
QVQLVESGGGVVQPGESLKLSCAASGRTFSSAIMGWFRQAPGKEREFVARITWSGAGVFYADSVKGRFTISRDNAKNTVYLQMNSLKPEDTAVYYCAAQALSQGDFGSWGQGTQVTVSSHHHHHHEPEA

SEQ ID NO: 4 - protein, 141 aa, Artificial Sequence
  CA17441 FBA1 binder amino acid sequence
QVQLVESGGGLVQPGGSLRLSCAASGFTLDYYAIGWFRQAPGKEREGVSCISSSDGSTYYADSVKDRFTISRDNAKNTVYLQMNSLKPEDTAVYYCAAVTEGVGAGNCGVSDLDGYGMDYWGKGTQVTVSSHHHHHHEPEA

SEQ ID NO: 5 - protein, 129 aa, Artificial Sequence
  CA17442 FBA1 binder amino acid sequence
QVQLVESGGGLVQTGGSLRLSCAASGRTFSRTIMGWFRQASGKEREFLARITWSGAGTFYADSVKGRFTISRDNAKNTVYLQMNSLKPEDTAVYYCAAQGLDQRDFSSWGQGTQVTVSSHHHHHHEPEA

SEQ ID NO: 6 - protein, 128 aa, Artificial Sequence
  CA17443 FBA1 binder amino acid sequence
QVQLVESGGGSVQPGGSLRLSCAASGNILRIYSMGWSRQAPGKQRELVASITRAGSTRYADSVKGRFTISRDSTNNMVYLQMNSLEPEDTAVYFCNANPVYDGSAGYWGQGTQVTVSSHHHHHHEPEA

SEQ ID NO: 7 - protein, 123 aa, Artificial Sequence
  CA17451 PDC1 binder amino acid sequence
QVQLVESGGGLVQPGGSLRLSCAASGISFSINTLGWYRQAPGKQRELVATVTTGGSTYYVDSAKGRFTVSRDNAKNTVYLQMNSLNSEDTAVYYCKLERYGVWGQGTQVTVSSHHHHHHEPEA

SEQ ID NO: 8 - protein, 130 aa, Artificial Sequence
  CA17452 PDC1 binder amino acid sequence
QVQLVESGGGLVQAGASLTLSCAASGRTFNTYSMHWFRQAPGKEREFVAATNWGGNSKFYADSVRGRFTISRDNTKNMVYLQMNSLKPEDTAVYYCAAHRTVVGKTPDYWGQGTQVTVSSHHHHHHEPEA

SEQ ID NO: 9 - protein, 132 aa, Artificial Sequence
  CA17444 SIS1 binder amino acid sequence
QVQLVESGGGLVQPGGSLRLSCAASGFTLDYYAIGWFRQAPGKEREGVSCISSSDGSTVYADSVKGRFTISRDNAKNTVYLQMSNLKPEDTAVYYCATDQITSTGCPDFDSWGQGTQVTVSSHHHHHHEPEA

SEQ ID NO: 10 - protein, 124 aa, Artificial Sequence
  CA17453 ALD6 binder amino acid sequence
QVQLVESGGGVVQPGGSLRLSCAASGRIFIRSAWYRQAPGKQRALVASIFSGGTTNYADSVKGRFTISRDNAKNTVILEMNNLKPEDTAVYYCNVRVRSGTDYWGQGTQVTVSSHHHHHHEPEA

SEQ ID NO: 11 - protein, 124 aa, Artificial Sequence
  CA17454 ALD6 binder amino acid sequence
QVQLVESGGGLVQPGGSLRLSCAASGSIFSRNYIAWYRQAPGKQRELVARKTQSGATVYADSVKGRFTISTNNAKNTMYLQMNILKPEDTAVYYCNAGGLIGSWGQGTQVTVSSHHHHHHEPEA

SEQ ID NO: 12 - protein, 121 aa, Artificial Sequence
  CA17460 ALD6 binder amino acid sequence
QVQLVESGGGLVQPGDSLRLSCAASGFTVSTNTMTWVRQAPGKGLEWVSTKYSTGGTTYANSVKGRFTISRDNAKNTLYLQMNNLKPEDTAVYYCGGADYRGQGTQVTVSSHHHHHHEPEA

SEQ ID NO: 13 - protein, 125 aa, Artificial Sequence
  CA17458 BMH1 binder amino acid sequence
QVQLVESGGGLVQPGGSLRLSCAASGTTLSLRRVGWYRQAPGKQRDLVATLTSGGETSYADSVKGRFTISRDNAKNTVYLQMNSLKLEDTAVYYCNAINWPMDYWGQGTQVTVSSHHHHHHEPEA

SEQ ID NO: 14 - protein, 136 aa, Artificial Sequence
  CA17459 BMH1 binder amino acid sequence
QVQLVESGGGEVQTGGSLRLSCTASGSRLNFGSWNWYRKRGENYRELVAAMGPSTGHTHYGDSMKGRVTISLNAAKDTLFLQMNSLKPEDTAVYYCAADLDGLGFPRGSFYGMDYWGKGTQVTVSSHHHHHHEPEA

SEQ ID NO: 15 - protein, 128 aa, Artificial Sequence
  CA17455 PGI1 binder amino acid sequence
QVQLVESGGGLVQPGGSLRLSCAASGITFPSYRLSWYRQAPGKERELVAAITSDGGTTNYADFVKGRFTISRDNTKNTVYLQMNSLKPEDTAVYYCKAQYFSTPLTYWGQGTQVTVSSHHHHHHEPEA

SEQ ID NO: 16 - protein, 129 aa, Artificial Sequence
  CA17456 PGI1 binder amino acid sequence
QVQLVESGGGLVQPGGSLRLSCAASRSIVTINVMGWYRQAPGKEREFVAGATSGGSTDYADSVKGRFTISRDNAKNTVYLQMNSLKPEDTAVYYCNLRGTGYNYGENYWGKGTQVTVSSHHHHHHEPEA

SEQ ID NO: 17 - protein, 142 aa, Artificial Sequence
  CA17457 PGI1 binder amino acid sequence
QVQLVESGGGLVQTGGSLRLSCAASGGHFSNLAMGWFRQAPGKEREFVAAIGRSQFNLLYSNFVKGRFTISRDNAENTVYLQLNSLNSEDTAIYYCASSGGVSDWRGIMTTGARLARNYDYWGQGTQVTVSSHHHHHHEPEA

SEQ ID NO: 18 - protein, 133 aa, Artificial Sequence
  CA17530 SXM1 binder amino acid sequence
QVQLVESGGGLVQTGGSLRLSCVVSKTLFSINAMAWYRQSPGKPRELVAGWTSGGRTSYGDSVKGRFTISRDNAENTLYLQMNDVKPEDTAVYHCAADYLVVAGKPGYDYAYWGQGTQVTVSSHHHHHHEPEA

SEQ ID NO: 19 - protein, 137 aa, Artificial Sequence
  CA17560 SSA1 binder amino acid sequence
QVQLVESGGGLVHSGGSLRLSCVASGGAFNDYTMGWFRQAPGKERQFVAAITWSSGVTSYSDSVRGRFVISRDIAKNTVYLQMNSLNPEETAVYVCAARLGVSSSPNSQISHRYDYWGQGTQVTVSSHHHHHHEPEA

SEQ ID NO: 20 - protein, 137 aa, Artificial Sequence
  CA17561 SSA1 binder amino acid sequence
QVQLVESGGGLVQAGGSLRLSCAASTRTFSNYVMAWFRQAPGKEREFVGAINWSGGTEYYADSVKGRFTISRDNAKNTVYLQMNDLNPEDTANYYCAARRGTIARTALVKYLEYDYWGQGTQVTVSSHHHHHHEPEA

SEQ ID NO: 21 - protein, 120 aa, Artificial Sequence
  CA17562 SSA1 binder amino acid sequence
QVQLVESGGGLVQPGGSLRLSCAASGFDFSRNRLNWHRQAPGKQRELVATIGSNGPRYADSVKDRFTISRDNEKNTVYLEMNSVKPEDTAVYYCAARGYWGQGTQVTVSSHHHHHHEPEA

SEQ ID NO: 22 - protein, 131 aa, Artificial Sequence
  CA17563 SSA1 binder amino acid sequence
QVQLVESGGGTVQAGDSLRLSCSASGRALTPYTMAWFRQPPGKERELVAAIGRTGSGTRYAEAVKDRFTISRDNDKQELYLQMNSLKPEDTAVYYCAANRRLTTSLEYDYWGQGTQVTVSSHHHHHHEPEA

SEQ ID NO: 23 - protein, 134 aa, Artificial Sequence
  CA17564 SSA1 binder amino acid sequence
QVQLVESGGGLVQPGDSLRLSCVVSVQHFGNYIVGWFRQPPGKEREFVSAISRGLRTYYADSVKGRVTISRDNAKNSVYLQMNSLKPEDTAVYYCAGSRLLEGAPRDSRGYNNWGQGTQVTVSSHHHHHHEPEA

SEQ ID NO: 24 - protein, 126 aa, Artificial Sequence
  CA17565 SSA1 binder amino acid sequence
QVQLVESGGGRVQPGGSLRLSCVASGFTFTSYGMSWYRQTPGKEREFVAAISSESTWADYGDSVKGRFTISRDNAKKMVYLQMNSLKPDDTAVYYCNRDQVPWPTWGPGTQVTVSSHHHHHHEPEA

SEQ ID NO: 25 - protein, 238 aa, Artificial Sequence
  GFP protein
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 26 - protein, 139 aa, Artificial Sequence
  CA17517 GFP nanobody amino acid sequence
QVQLVESGGGLVQPGGSLRLSCAASGRTFSNYIMGWFRQPPGKEREFVATISWSGSDTVYASSVKGRFTISRDNAKSTVYLQMNSLKPEDTAIYYCAAKDAVGYWSSDVYSEAKEYDYWGQGTQVTVSSHHHHHHEPEA

SEQ ID NO: 27 - protein, 140 aa, Artificial Sequence
  CA17518 GFP nanobody amino acid sequence
QVQLVESGGGLVQAGGSLRLSCVTSGFTFDDYAIGWFRQAPGKEREGVSSISSSDGTTYYADSVKGRFTISSDNAKNTVYLQMNNLKPEDTAVYYCAADWAAWPSIRVGVGVPAYEYDYWGQGTQVTVSSHHHHHHEPEA

SEQ ID NO: 28 - protein, 131 aa, Artificial Sequence
  CA17519 GFP nanobody amino acid sequence
QVQLVESGGGLVQPGGSLRLSCAASGRIFSIYVMGWYRQAPGKQRELVAQITNSGRTKFADSVKGRFTISRDNAKNMVYLQMNSLKAEDTAVYYCYADLDWYGGTANNDFWGQGTQVTVSSHHHHHHEPEA

SEQ ID NO: 29 - protein, 139 aa, Artificial Sequence
  CA17520 GFP nanobody amino acid sequence
QVQLVESGGGLVQTGGSLRLSCAASVRTFSNYIMGWFRQAPGKEREFVATISWSGINTYYADNVKGRFTISRDNARNTVYLQMNSLKPEDTAIYYCAAKSAVGYYHDSYASRAEEYDYWGQGTQVTVSSHHHHHHEPEA

SEQ ID NO: 30 - protein, 139 aa, Artificial Sequence
  CA17673 GFP binder amino acid sequence
QVQLVESGGGLVQAGGSLRLSCAASARAFMNYIMGWFRQAPGKEREFVATISWSGVTTDYADNVKGRFTISRDNAKNTVSLQMNSLKPEDTAIYYCAAKSAVGYYSDIYNSRAKEYDYWGQGTQVTVSSHHHHHHEPEA

SEQ ID NO: 31 - protein, 139 aa, Artificial Sequence
  CA17674 GFP binder amino acid sequence
QVQLVESGGGLVQPGDFLRLSCAASDRTFSNYIMGWFRQAPGKEREFVATISWSGSDTVYASSVKGRFTISRDNAKSTVYLKMNSLKPEDTAIYYCAAKYAVGYWGNDVASEAKDYNYWGQGTQVTVSSHHHHHHEPEA

SEQ ID NO: 32 - protein, 131 aa, Artificial Sequence
  CA17675 GFP binder amino acid sequence
QVQLVESGGKLVPAGDSLRLSCAASGRTFSDYIIGWFRQVSGKERKFVAAIRRSDGETVYTDSVKGRFTISRDNAKNTVYLQMNSLKPEDTAVYYCYLNNRNSRGSMDAYWGQGTQVTVSSHHHHHHEPEA

SEQ ID NO: 33 - protein, 140 aa, Artificial Sequence
  CA17676 GFP binder amino acid sequence
QVQLVESGGGLVQAGGSLRLSCAGSGGTFSAYTMGWFRQGLGEERELVACISRSGASTFYTDPVKGRFTISRDNDKNTVYLQMNNLKPEDTAVYYCAADWTVIDEITEQRSKHQTSYDYWGQGTQVTVSSHHHHHHEPEA

SEQ ID NO: 34 - protein, 124 aa, Artificial Sequence
  CA8780 irrelevant binder amino acid sequence
QVQLQESGGGLVQPGGSLRLSCAPSGPFSPNSMGWYRQAPGKQRELVAVMTIDGRTNYQDSVKGRFTISRDYVKNTAYLQMNNLKPDDTAVYICNAETRGFMHWGQGTQVTVSSHHHHHHEPEA

SEQ ID NO: 35 - protein, 139 aa, Artificial Sequence
  CA17797 GR-LBD binder amino acid sequence
QVQLVESGGGLVQAGDSLRLSCTASGRSSSSDYAMGWFRQAPGKEREFVSAIRWDTALKYYGHSVKGRFAVSRDKATNTVYLQMNSLKPEDTAIYYCAAAATFLQTAAEATSATRYSYWGQGTQVTVSSHHHHHHEPEA

SEQ ID NO: 36 - protein, 139 aa, Artificial Sequence
  CA17798 GR-LBD Binder amino acid sequence
QVQLVESGGGVAQAGGSLRLSCAASGLSFFTRTMGWFRQAPGKEREFVAAIGDSRRTHYADPVKGRYTLSRDNAKNTATLQMNSLKPDDTAVYYCAAALITPETMVEPLSSEAWTFGSWGQGTQVTVSSHHHHHHEPEA

SEQ ID NO: 37 - protein, 136 aa, Artificial Sequence
  CA17799 GR-LBD Binder amino acid sequence
QVQLVESGGGLVQPGDSLKLSCAASGRTFTNYAIGWFRQAPGKERAFVSAISASGRSTYYADFVKGRFTVSRDNANKTVYLQMDNLQPEDTATYYCAHAQSIPQLKRKNPHDSDYWGQGTQVTVSSHHHHHHEPEA

SEQ ID NO: 38 - protein, 136 aa, Artificial Sequence
  CA17800 GR-LBD Binder amino acid sequence
QVQLVESGGGLAQVGGSLSVSCAASGRPFDNYGMGWFRRKPGLDYEFVSHITWDGEYTRYSDAVKGRFFVSRDNAKSTVTLKMNNLQPEDTAVYFCAAKAKTNTRPYYYEENYSYWGQGTQVTVSSHHHHHHEPEA

SEQ ID NO: 39 - protein, 130 aa, Artificial Sequence
  CA17801 GR-LBD Binder amino acid sequence
QVQLVESGGGLVQPGGSLRLSCVVPGSVRSNIDMAWFRQAQGKQRELVASITGDGTTNYVDSVRGRFIISRDNDKRTMYLQMNSLKPEDTAVYYCNANRMTGWGAKRDYWGQGTQVTVSSHHHHHHEPEA

SEQ ID NO: 40 - protein, 122 aa, Artificial Sequence
  CA18504 HSP104 Binder amino acid sequence
QVQLVESGGGLVQPGGSLTLSCAGSGRAFDSIAMGWFRQAPGKEREFVAAISWNGVSAYYADSVKGRFTISRDNAKNTVHLQMNSLQSDDTAVYYCHADNSWGQGTQVTVSSHHHHHHEPEA

SEQ ID NO: 41 - protein, 132 aa, Artificial Sequence
  CA18505 MET6 Binder amino acid sequence
QVQLVESGGGLVQPGGSLRLSCAASGSIFSINAWGWYRQAPGKERELVAVITSVGGRTNYADSVKGRFTISRDNAKDMVYLQMNSLKPEDTAVYYCNSRGSSWYFGTPQDYWGQGTQVTVSSHHHHHHEPEA

SEQ ID NO: 42 - protein, 134 aa, Artificial Sequence
  CA18508 SBA1 Binder amino acid sequence
QVQLVESGGGLVQPGGSLRLSCAASGFTFSNTAMSWYRQAPGKERELVAVSTSAGGGTDYADSVQGRFTISRDNAKNTVYLQMNDLKPEDTAVYYCYAVGRYRSSWRLLPFGSWGQGTQVTVSSHHHHHHEPEA

SEQ ID NO: 43 - protein, 128 aa, Artificial Sequence
  CA18509 SBA1 Binder amino acid sequence
QVQLVESGGGLVQPGGSLRLSCAAAGFTFRMLDMSWYRQAPGKERELVATITSVSGTTNYADSVKGRVTISRDNAKNTVYLQMNSLKPEDTAVYYCNARMAAWGLDYWGQGTQVTVSSHHHHHHEPEA

SEQ ID NO: 44 - protein, 134 aa, Artificial Sequence
  CA18510 SOD1 Binder amino acid sequence
QVQLVESGGGLVEAGGSLRLSCAASGRTFSSYAMGWFRQAPGKEREFVAAISWSGGSTYYADSVKGRFTISRDNAKNTVYLQMNSLKPEDTAVYYCAAPTAYHYSTTRDEYDSWGQGTQVTVSSHHHHHHEPEA

SEQ ID NO: 45 - protein, 131 aa, Artificial Sequence
  CA17938 ENO1 Binder amino acid sequence
QVQLVESGGGLVQPGGSLRLSCAASGFTLDTWAIGWFRQVPGKDREGAACISSEPSSTYYGEFVNGRFTISRDSAKSTVYLQMNDLKPEDTAVYYCSADRLSAYCSGSGGWGQGTQVTVSSHHHHHHEPEA

SEQ ID NO: 46 - protein, 127 aa, Artificial Sequence
  CA17791 PGI1 Binder amino acid sequence
QVQLVESGGGLVQPGGSLRLSCAASGITFSSYAMGWYRQAPGKERELVARITSGGSTDYAVSVKGRFTISRDNANNLVYLQMNSLNSEDTAVYYCHSQYSYGPLEYWGQGTQVTVSSHHHHHHEPEA

SEQ ID NO: 47 - protein, 128 aa, Artificial Sequence
  CA17792 PGI1 Binder amino acid sequence
QVQLVESGGGLVQPGGSLRLSCAASGISFGSYTMAWYRQAPGKQRDMVADITTGGRTNYADSVKGRFTISRDNAKNTVYLQMNSLKSEDTAVYYCKADFAYGRDLTYWGQGTQVTVSSHHHHHHEPEA

SEQ ID NO: 48 - protein, 127 aa, Artificial Sequence
  CA17793 PGI1 Binder amino acid sequence
QVQLVESGGGLVRPGGSLRLSCAASGITFSSYTMGWYRQAPGKQRELVATITTGGSTNYAVSAKGRFTISRDNANNRVTLQMNSLNSEDTAAYYCHSRFAYGPLEYWGQGTQVTVSSHHHHHHEPEA

SEQ ID NO: 49 - protein, 129 aa, Artificial Sequence
  CA17794 PGI1 Binder amino acid sequence
QVQLVESGGGLVQPGGSLRLSCTASGVTFSRYAMSWYRQAPGKERELVATITGAGSSTNYEDSVKGRFTISRDNAKNTVYLQMNSLKPEDTAVYYCYADHYGLGYLDYWGQGTQVTVSSHHHHHHEPEA

SEQ ID NO: 50 - protein, 132 aa, Artificial Sequence
  CA17795 PGI1 Binder amino acid sequence
QVQLVESGGGLVQPGGSLRLSCAASGSFFNINAMAWYRQAPGKQRELVAVIASSGRTNYADSVSGRFTISRDNAKNTVYLQMNSLKPEDTAVYYCNSKIRQASWNLVLSDHWGQGTQVTVSSHHHHHHEPEA

SEQ ID NO: 51 - protein, 127 aa, Artificial Sequence
  CA17796 PGI1 Binder amino acid sequence
QVQLVESGGGLVQPGGSLRLSCAASGISFHSYAMGWYRQAPGKQRELVSTITTGGSTDYAVSVKGRFTISRDNAKNTVYLQMNNLNSEDTAVYYCHSQYAYGPLEYWGQGTQVTVSSHHHHHHEPEA

SEQ ID NO: 52 - protein, 132 aa, Artificial Sequence
  CA17875 VGLUT1 Binder amino acid sequence
QVQLQESGGGLVQAGGSLRLSCAASGRSVRTWSMGWFRQPPGKERELVAGISWSGSGTYHADSVKGRFTISRDNAKNMVYLQMNSLKPEDTAVYYCAAGQTGWNPGSDFHRWGKGTQVTVSSHHHHHHEPEA

SEQ ID NO: 53 - protein, 134 aa, Artificial Sequence
  CA18425 VGLUT1 Binder amino acid sequence
QVQLQESGGGLVQAGDSLRLSCAASGRTFSTTGMGWFRQAPGKEREFVAAVSWRSGNTYYADSVKGRFTISRDNAKTTVFLQMNSLKPEDTAVYYCAANPTGSYGAATSRYNYWGQGTQVTVSSHHHHHHEPEA

SEQ ID NO: 54 - protein, 131 aa, Artificial Sequence
  CA18024 VGLUT1 Binder amino acid sequence
QVQLVESGGGLVQAGGSLRLSCAVSGSIFRANAMGWYRQAPGKRRELVARITRGGFTNYVDSVKGRFTISRDNVKNTVYLQMSTLKPEDTAVYYCYAGIRQIRVSYEQDYYGQGTQVTVSSHHHHHHEPEA

SEQ ID NO: 55 - protein, 137 aa, Artificial Sequence
  CA18437 rVGLUT1 Binder amino acid sequence
QVQLVESGGGLVQAGDSLRLSCAALGNSFSTYAIGWFRQAPGKEREFVGAVNWRGDITLYADSVKGRFTISRDNAKDTVYLQMNSLKPEDTAVYYCAADETTQPMGITLSPNDYDYWGQGTQVTVSSHHHHHHEPEA

SEQ ID NO: 56 - protein, 131 aa, Artificial Sequence
  CA18438 rVGLUT1 Binder amino acid sequence
QVQLVESGGGLVQPGGSLRLSCAASRSIFAINKMGWYRQVPGKQRELVADITSGGSTNYADSVKGRFTISNDNAKNTVYLQMNSLKPEDTAVYYCNAILDSDYGEDEYDYWGQGTQVTVSSHHHHHHEPEA

SEQ ID NO: 57 - protein, 131 aa, Artificial Sequence
  CA18439 rVGLUT1 Binder amino acid sequence
QVQLVESGGGLVQPGGSLRLSCVASGSIFSINLMAWYRQAPGKQREIIAVITSGGSTNYADSVKGRFTISRDNAKNTGYLQMNSLEPEDTAVYYCNAHIATVSEGYSRPYWGQGTQVTVSSHHHHHHEPEA

SEQ ID NO: 58 - protein, 132 aa, Artificial Sequence
  CA18440 rVGLUT1 Binder amino acid sequence
QVQLVESGGGLVQPGGSLRLSCKASGFTFSNYVMTWVRQAPGKGLEWVSHINGGGGTTAYSDSVKGRFTVSRDNAENTLYLQMGSLKPEDTAVYYCAESSIGNVWAPSYDYWGQGTQVTVSSHHHHHHEPEA

SEQ ID NO: 59 - protein, 127 aa, Artificial Sequence
  CA18441 rVGLUT1 Binder amino acid sequence
QVQLVESGGGLAQVGGSLRLSCVASGSSFSGDVMGWYRQPPGQQRELVARISSNGRRTIAESVKGRFTIGRDNAKNTIYLQMDSLRPQDTAVYYCNTNSRETGIDYWGQGTQVTVSSHHHHHHEPEA

SEQ ID NO: 60 - protein, 134 aa, Artificial Sequence
  CA16964 mCherry-trapper Binder amino acid sequence
QVQLVESGGGLVQAGGSLRLSCAASGRDFSFDGMGWFRQAPGKEREFVAAISWSGLRIGYSDSVKGRFTISRDNAETTVHLQMNSLKPEDTAVYYCATGSHATTKAQLYDYEYWGQGTQVTVSSHHHHHHEPEA

SEQ ID NO: 61 - protein, 136 aa, Artificial Sequence
  CA17302 mCherry-stripper Binder amino acid sequence
QVQLVESGGGLVQAGGSLRLSCVASGLPFSRYNMAWFRQAPGKEREFVADIDWSGDSTYDLDSVKGRFTISRDNAKNTVYLQMSSLLPEDTAVYYCAAHFKFILAQRIKWGDMDYWGKGTQVTVSSHHHHHHEPEA

SEQ ID NO: 62 - protein, 125 aa, Artificial Sequence
  CA18498 GR Binder amino acid sequence
QVQLVESGGGVVQPGGSLRLSCAASGSRFSRYHMGWYRQAPGKQRELVALITSVDRTKYADSVKGRFTISRDNAKNMMYLQMNSLKLEDTAVYYCKLRKGGADYWGQGTQVTVSSHHHHHHEPEA

SEQ ID NO: 63 - protein, 125 aa, Artificial Sequence
  CA18499 GR Binder amino acid sequence
QVQLVESGGGLVQPGGSLRLSCAASGFTFSSYPMNWVRQAPGKGLEWVSRITPGGVYRTYAASVKGRFTISRDNAKNMLYLQMNTLKPEDTAVYYCTKDGAVRPRGQGTQVTVSSHHHHHHEPEA

SEQ ID NO: 64 - protein, 131 aa, Artificial Sequence
  CA18501 GR Binder amino acid sequence
QVQLVESGGGLVQAGGSLRLSCAASASASIFRSNVMGWYRQPPGNQRELVATITAGGSPNYADSVKARFTISRDNAKNIVYLQMNDLKPEDTAVYYCNLRSRSSYPYMDYWGKGTQVTVSSHHHHHHEPEA

SEQ ID NO: 65 - protein, 128 aa, Artificial Sequence
  CA18502 GR Binder amino acid sequence
QVQLVESGGGSVQAGGSLRLSCAASGNTASIRTMGWYRQAPGKRRELVAAFTSAGSINYEDSVKGRFTISRNNAKDTVYLQMNSLKPEDTAVYYCNLQQYERHLKDYWGQGTQVTVSSHHHHHHEPEA

SEQ ID NO: 66 - protein, 130 aa, Artificial Sequence
  CA18503 GR Binder amino acid sequence
QVQLVESGGGMVQPGGSLRLSCAASGFTFSKYDMNWVRQAPGKGPEWVSTIRRGGDVTYYADSVKGRFTISRDNAKNTVYLQMSSLKPDDTAVYYCKADRPGWGSEWEYWGQGTQVTVSSHHHHHHEPEA

SEQ ID NO: 67 - protein, 135 aa, Artificial Sequence
  CA18585 GR Binder amino acid sequence
QVQLVESGGGLVQPGGSLRLSCAASGFAFGIFNNYHMSWFRQAPGKERELAALISGIGTVTNYAASVKGRFTISRNNTKNTVSLQMNSLEAEDTAVYFCGGGRPSSRSPYGMDYWGKGTQVTVSSHHHHHHEPEA

SEQ ID NO: 68 - protein, 127 aa, Artificial Sequence
  CA18586 GR Binder amino acid sequence
QVQLVESGGGLVQAGGSLRLSCAASGRNLYMGIMGWYRQAPGKARELVATITRGGSTNYADSVKGRFTVSRDNAKRTVYLQMDTLKPEDTAIYYCGSNSGTYSLRFWGQGTQVTVSSHHHHHHEPEA

SEQ ID NO: 69 - protein, 10 aa, Artificial Sequence
  6xHis + EPEAtag
HHHHHHEPEA

SEQ ID NO: 70 - protein, 126 aa, Artificial Sequence
  CA12760 GFP-stripper amino acid sequence
QVQLVESGGGLVQAGGSLRLSCAASGRTFSTAAMGWFRQAPGKERDFVAGIYWTVGSTYYADSAKGRFTISRDNAKNTVYLQMDSLKPEDTAVYYCAARRRGFTLAPTRANEYDYWGQGTQVTVSS

SEQ ID NO: 71 - protein, 126 aa, Artificial Sequence
  CA15816 GFP-trapper amino acid sequence
QVQLVESGGGLVQAGGSLRLSCAASGRTFSTAAMGWFRQAPGKERDFVAGIYWAAGSTYYADSAKGRFTISRDNAKNTVYLQMDSLKPEDTAVYYCAARRRGFTLAPTRANEYDYWGQGTQVTVSS

(19) United States
(12) Patent Application Publication
(10) Pub. No.: US 2024/0287501 A1
(43) Pub. Date: Aug. 29, 2024

(54) Means and Methods for Selection of Specific Binders

(71) Applicants: VIB VZW, Ghent (BE); Vrije Universiteit Brussel, Brussel (BE)

(72) Inventors: Jan Steyaert, Beersel (BE); Els Pardon, Wezemaal (BE); Alexandre Wohlkönig, Brussel (BE); Thomas Zögg, Heverlee (BE); Valentina Kalichuk, Kraainem (BE)

(21) Appl. No.: 18/571,904

(22) PCT Filed: Jun. 23, 2022
(86) PCT No.: PCT/EP2022/067256

§ 371 Date: Dec. 19, 2023

(30) Foreign Application Priority Data
Jun. 23, 2021 (EP) 21181272.2
Jun. 24, 2021 (EP) 21181405.8

(51) Int. Cl. C12N 15/10

(52) U.S. Cl. CPC C12N 15/1037

(57) ABSTRACT

The disclosure relates to a novel method for selection and identification of specific polypeptide binding agents for a target of interest. More specifically, the selection method involves capturing the target of interest on a surface using a first binding agent to form an immobilized antigen complex, selecting for specific antigen-binding polypeptides present in a sample, preferably as a display library, and eluting the target protein and the selective polypeptide binder, using a second binding agent competing for the target binding site of the first binding agent. More specifically, the selection method presented herein provides for an efficient and highly selective medium- to high-throughput technology applicable to recombinant antibody libraries, including immune and unbiased or proteome-wide display libraries, wherein selections can be performed without a need for purified target protein, in physiological conditions.

### CROSS-REFERENCE TO RELATED APPLICATIONS

This application is a national phase entry under 35 U.S.C. § 371 of International Patent Application PCT/EP2022/067256, filed Jun. 23, 2022, designating the United States of America and published in English as International Patent Publication WO 2022/268993 on Dec. 29, 2022, which claims the benefit under Article 8 of the Patent Cooperation Treaty to European Patent Application Serial No. 21181272.2, filed Jun. 23, 2021, and to European Patent Application Serial No. 21181405.8, filed Jun. 24, 2021, the entireties of which are hereby incorporated by reference.

### FIELD OF THE INVENTION

The disclosure relates to a novel method for selection and identification of specific polypeptide binding agents for a target of interest. More specifically, the selection method involves capturing the target of interest on a surface using a first binding agent to form an immobilized antigen complex, selecting for specific antigen-binding polypeptides present in a sample, preferably as a display library, and eluting the target protein and the selective polypeptide binder, using a second binding agent competing for the target binding site of the first binding agent. More specifically, the selection method presented herein provides for an efficient and highly selective medium- to high-throughput technology applicable to recombinant antibody libraries, including immune and unbiased or proteome-wide display libraries, wherein selections can be performed without a need for purified target protein, in physiological conditions.

### Introduction

Over the years, a multiplicity of display and selection methods have been reported and widely applied as antibody discovery techniques, providing for means and methods to identify and isolate novel polypeptide binders, mainly monoclonal antibodies (mAbs), antibody fragment, or alike, starting from large collections. The production of monoclonal antibodies requires hybridoma cell lines, i.e. hybrid cell lines produced by the fusion of an antibody-producing lymphocyte with a tumor cell. The hybridoma technology, as pioneered in the 1970s by Kohler and Milstein, still remains state-of-the-art in antibody hit discovery. Though, the cloning of antibody genes as single domains or as fragments in combinatorial libraries found its way to more and higher throughput approaches for antibody discovery. The principle of genotype-phenotype coupling was born by this development, since this ‘barcoding’ was key for up to several billion different protein variants of which binders can be selected for via high-throughput identification in an iterative process. Several display technologies, but especially the expression of functional antibody fragments on the surface of filamentous phage, called phage display, used for in vitro selection of antibody fragments by McCafferty and Chiswell in Cambridge, Barbas in La Jolla as well as Breitling and Dübel in Heidelberg, revealed a faster and more efficient selection process, which in combination with clone identification set the basis for antibody screening platforms. Besides phages, selection methods using nonphage-display systems have been established, such as ribosome and mRNA-display, which are well-suited for purified antigen, as well as yeast display libraries, often applied in cell sorting-based selection methods, and even mammalian display, which benefits to therapeutic developments to apply a similar host, though slow growth rates are to be accounted for. For a recent review on the different approaches see for instance Valdorf et al. (2021).

Recombinant antibody libraries, universal libraries, naïve (non-immune) or immune-based libraries are applied for high-throughput selection of target-specific binders. Whereas immune-libraries are intended for specific target-selection, the so-called ‘universal libraries’ can be sub-divided into naïve, synthetic and semi-synthetic approaches allowing unbiased selection of novel binders (also see Almagro et al. 2019). After obtaining individual clones from iterative selection rounds, target-binding and biophysical properties of the polypeptide binders are screened for using typical antibody-binding assays, ranging from ELISAs to immunoprecipitation. Alternative selection methods evolving from the ‘golden standard’ for selection of recombinant display libraries are often based on customized needs, such as reported by Lakzaei et al., (2018), disclosing a biopanning method for Diphteria toxin binders, using soluble-antibody capturing which allows to capture binders specifically recognizing native epitopes, though still requiring acidic elution conditions, and without any control of selecting for specific conformations of interest. Another example concerns the Yin-Yang biopanning method which provides for an affinity selection against crude extracts, thereby solving the problem of antigen purification needs, and obtained specificity using negative selection (blocking agents) prior to positive phage selection (Lim et al. 2019).

The application of display technologies enabling the selection of Abs with therapeutic relevance have laid the basis for a vast number of market-approved therapeutics either by US, European or Chinese healthcare authorities. This observation supports the valuable impact those platform technologies have with respect to drug discovery. Moreover, combining display technologies with microfluidic systems and/or next-generation sequencing in antibody discovery has been enabling for the implementation of functional screenings, for instance, thereby further enriching the toolbox and pipeline for high throughput selection methods in therapeutic areas. Conventional methods applying phage display, or alternatives and further evolved platforms nowadays provide for high-tech and commonly applied display technologies to select for polypeptide binders, especially from recombinant antibody libraries, with great potential in drug discovery. However, there is still room for improvement to tackle certain technical bottlenecks such as the use for difficult-to-purify antigens under native conditions, for selection of binders against conformational epitopes of target antigens, and for increasing efficiency and specificity over the background when applied in high-throughput mode.

### SUMMARY OF THE INVENTION

The present disclosure is based on the finding that the NANEX technology method (as described further herein) is applicable for selecting and screening recombinant antibody libraries in physiological conditions, without a need for purified antigen.

Nanobody-based exchange chromatography (also referred to as NANEX) provides for a Nanobody (Nb)-based immune-displacement purification method wherein a pair of Nbs (a trapper as capturing agent and a stripper as eluting agent), competing for binding to the same or highly overlapping epitope on a target is used to analytically purify this target protein yielding small amounts of highly pure protein bound to the high affinity Nb (stripper) (as described previously in PCT/EP2020/087291). Such a highly-selective one-step purification method has the advantages of not requiring a concentration step, dialysis or proteolysis, and that physiological conditions can be maintained during protein capture and elution. Moreover, in cases where only one specific Nb is available, the use of Nbs as antigen-binding moieties in the NANEX purification method also allows one to start from a single binder, to obtain a pair via design of a lower affinity or faster dissociating variant (e.g. a mutant).

The present application describes the proof-of-concept demonstrating that the integration of the NANEX purification method in display library selections provides for a powerful approach with improvement of selection procedures to screen for binders in mild conditions, even for intractable protein targets. Even when unbiased Nb libraries were used for selection, specific high affinity target binders were identified in a very efficient manner. NANEX-based trapping of a target protein allows for the maintenance of native physiological conditions for the target, releases the need for purified target or antigen, since the target can be provided as a complex sample (e.g. a biological sample), and resulted in a novel innovative antibody selection process that is capable of phishing out potent binders from polypeptide binders raised against a complete proteome of targets. This is not possible in such an efficient manner using conventional selection procedures, and therefore positions the NANEX-based panning or selection method as a breakthrough in drug discovery of challenging targets or tool generation. More specifically, after only 2-3 rounds of selection, a significant robust enrichment for the different antigens was demonstrated using this selection approach, and moreover, the success was independent of their cellular abundance when selected from a proteome-wide immune library. Also, several families of native protein binders could be identified, even with few Nb family members (which often disappear after 3 rounds in conventional selection). In addition to selection of binders for soluble protein targets, we further also demonstrated proof of concept for membrane protein binder screening, and robust isolation of novel binders, specifically shown herein for Nb families, that may even be overlooked in conventional selection methods was demonstrated.

Although immune libraries against a plurality of proteins have been raised in llamas in the past (e.g. against a T. evansi secretome in Li et al., 2020), we surprisingly found that using a proteome-wide Nb library, NANEX-purified phage display selection resulted in target specific binders against the majority of tested targets, thereby demonstrating the selective power and reliability of this novel selection method. An additional benefit is that the requirement for purified antigen is avoided throughout the whole process, when making use for instance of endogenously GFP-tagged yeast clones in combination with GFP-specific NANEX, or of a trapper/stripper pair against endogenous protein epitopes which is very attractive to implement in selections where difficult to purify antigens are at stake.

So, this selection method has the advantages that target purification and subsequent selection of specific target binders is possible under physiological and native conditions, while obtaining highly specific and thus selective target protein-polypeptide binder interaction, and/or conformation-specific target protein binders, all possible at high-throughput conditions. Moreover, when applied on tagged target proteins, such as the GFP-specific trapper/stripper shown herein, a generic selection platform using a single trapper/stripper (or capturer/displacer) pair is provided.

So a first aspect of the disclosure relates to a selection process for protein binders from a plurality of binders (e.g. a display library) specifically binding a target protein wherein NANEX is used to form an immobilized complex between the capturing agent (or first protein binding agent, also called trapper) and the target, which is presented to the plurality of potential binders, allowing for selecting a protein binder associated to said target at a different binding site than the capturing agent. Said complex is then eluted using the eluting agent (or second protein binding agent, also called stripper), which outcompetes the trapper for binding the target and elutes the target in association with the selected binder as a tight target-stripper complex. The advantage in such a NANEX-based selection procedure being that a target can be captured from a complex native environment while retaining a particular/native conformation, without need for purified target protein, and that selection allows for identification of more/alternative binders as compared to or complementary to conventional selection procedures.

Said selection method comprises the steps of:
a) providing an immobilized first protein binding agent which specifically binds the target protein and a sample comprising the target protein, for obtaining an immobilized complex of the target bound to said first protein binding agent,
b) adding to said immobilized complex of step a) a solution comprising a plurality of polypeptide binders, for allowing said polypeptide binders to specifically bind the immobilized target protein,
c) providing to said solution of step b) a solution comprising a second protein binding agent, which binds the target protein and competes for said binding with the first binding agent, thereby displacing the target protein, for releasing the target protein into the solution, and
d) collecting the eluate of the second protein binding agent bound to the target protein, for isolation of a polypeptide binder bound to said target protein.

A further embodiment relates to said method, wherein the second protein binding agent has a higher affinity and/or a rate constant of dissociation (k_{off} value) that is lower or equal as compared to the k_{off} value of the first binding agent. Another embodiment relates to said method wherein the second and/or first protein binding agent comprises an antigen-binding domain, which specifically binds the target protein. More specifically, the first and/or second agent antigen-binding domain comprises an immunoglobulin fold, is an antibody, or active antibody fragment, single domain antibody, an immunoglobulin single variable domain (ISVD), a VHH, a Nanobody, or an antigen-binding chimeric protein, which is defined as an ISVD fused to a scaffold protein via at least two sites, and preferably the scaffold protein domain comprising HopQ, YgjK, or a derivative thereof, also called a MegaBody.

Further embodiment of any of said methods described herein provide for the application of a plurality of polypeptide binders of step b) which are provided in the form of a display library of binding agents, which may more specifically comprise a recombinant antibody library, including immune libraries, or non-immune libraries, such as naïve libraries, including (semi-)synthetic libraries, expressing and displaying binding agents that are (monoclonal) antibodies, single domain antibodies, Fabs, ISVDs, VHHs or Nanobodies, or any active fragments thereof.

In a further specific embodiment, said method as described herein is performed using phage display, as known by the skilled person. Alternatively, a specific setup or library is applied to perform said method using yeast, ribosome, bacteria, or mammalian display.

Further specific embodiments aiming to enrich the number of specific polypeptide binders for the target protein provide for a method wherein a reiterative process of selecting the polypeptide binders is applied, in a similar manner as known in the art for panning procedures, wherein steps a) to d) are repeated at least once, preferably twice or more, wherein said reiterative process is indicated as ‘x rounds’ of selection. Specifically, when phage display libraries are used, the collected elution containing the phages in step d) is reused to infect E. coli and return to step a).

In a further embodiment, the method as described herein specifically comprises a surface with the first protein binding agent for immobilizing the target protein wherein said surface comprises a matrix comprising beads, as exemplified herein, such as magnetic beads, or alternatively said surface is provided by a resin, a chromatographic or polymeric column, a plate setup, or a microchip.

Another specific embodiment relates to said method wherein the target protein is present in step a) in a sample that is a complex sample, comprising a mixture of components, such as biological material, a cell lysate, an extract, a soluble proteome, a proteome of a certain cell- or tissue-type, or a recombinant target protein as part of mixture of proteins. A further specific embodiment provides for said selection method applying an immunogen for obtaining the plurality of polypeptide binders wherein the immunogen comprises the same nature and composition as the sample comprising the target protein in step a). For example, the immunogen comprises a cell lysate being applied in immunization to generate a display library such as a recombinant antibody library, wherein such a cell lysate is also applied in step a) as sample comprising the target protein.

An alternative method as described herein provides for a generic method, wherein the first and second protein binding agent specifically bind a tag, wherein said tag is present on the target protein, preferably as a N- or C-terminal heterologous tag. In a preferred embodiment, said heterologous tag is specifically recognized by GFP-specific binders, said embodiment relating to the selection method as described herein wherein the trapper comprises at least the CDRs, or the sequence of VHH of SEQ ID NO:71, and the stripper comprises at least the CDRs, or the sequence of VHH of SEQ ID NO:70.

A further embodiment of said method described herein allows for selection of polypeptide binders which specifically bind the eluted protein complex in step d) in a direct or indirect manner to the target protein. More specifically, direct binding to the target is obtained for polypeptide binders that recognize the target protein itself, at a binding site different from the trapper/stripper binding site, and indirect binding may be obtained by polypeptide binders which bind a tag fused to the target protein, or which bind another protein or component that is co-captured in step a) and is bound to the immobilized surface (via the target protein). Said indirect polypeptide binders may thus comprise binders specifically binding and recognizing proteins or components that are interactors of the target protein, or that are a heterologous fusion partner/tag of the target protein.

In another aspect the invention thus also relates to a protein complex comprising the second protein binding agent bound to the target protein, as eluted in step d), wherein said target protein is further bound to at least one additional protein, and wherein the method as described herein resulted in the presence of a polypeptide binder bound to said additional protein in said complex.

A further embodiment provides for the selection method as described herein, wherein parallel selection of at least two different target proteins is aimed for, by providing a sample in step a) comprising said at least two different target protein, separately or as a fusion or crosslinked molecule, and a first and second protein binding agent for each of these different target proteins is provided in step a) and c), respectively.

A further method for selecting a polypeptide binder specific for a target or antigen protein relates to an alternative approach of providing the mixture in step c) of the above described method and comprises the steps of:
a) mixing a target protein sample with a sample comprising a plurality of polypeptide binders, preferably a display library, and
b) obtaining an immobilized complex on a surface by adding to the mixture of a) a first protein binding agent, which is preferably immobilized on a surface or which is subsequently immobilized, and
c) providing to said solution of step b) a solution comprising a second protein binding agent, which binds the target protein and competes for said binding with the first binding agent, thereby displacing the target protein, for releasing the target protein into the solution, and
d) collecting the eluate of the second protein binding agent bound to the target protein, for isolation of a polypeptide binder bound to said target protein.

Specifically said alternative method may preferably use purified target protein sample. In a preferred embodiment of this method, the first and/or second protein binding agent comprise a Nanobody.

A final aspect of the present invention relates to the use of the method as described in any of the embodiment provided herein, for selection of binders from immune libraries. Alternatively, the use of the method as described in any of the embodiment provided herein, for epitope binning on a target protein, or for novel epitope binding agent isolation. Moreover, the use of the method as described herein is intended for high-throughput purposes in selection of protein binders, specifically in antibody and drug discovery.

### DESCRIPTION OF THE FIGURES

The drawings described are only schematic and are non-limiting. In the drawings, the size of some of the elements may be exaggerated and not drawn on scale for illustrative purposes.

FIG. 1. Schematic representation of the use of Nanobody exchange chromatography (NANEX) for the selection of target-specific antibodies from antibody display libraries.

The principle of Nanobody exchange chromatography can be applied for the selection of target specific binding agents like e.g. antibodies from display libraries. A first Nanobody (the trapper) that is covalently attached to a solid support (herein a bead) is used to immobilize a target. The immobilized target is then incubated with a diverse repertoire of different binding agents (e.g. antibodies) that are expressed and displayed in a way to provide a physical link between the phenotype (binding behavior) and the encoding genotype, in this case a phage display library. Washing steps can optionally be used to remove irrelevant binding agents or antibodies. Similar to NANEX and particular to this invention, a second Nanobody (a soluble stripper) that competes with the trapper is then used to selectively elute the immobilized target in association with the stripper and target-specific binding domains or antibodies and their encoding genotype.

FIG. 2. Enrichment after the first and second rounds of selection on GFP using NANEX.

Nanobody exchange chromatography (NANEX) was used for the selection of GFP-specific antibodies from a Nanobody display library according to Example 1. A GFP trapper (CA15816 SEQ ID NO:2) was coupled to Magnetic beads and different NANEX beads were prepared by incubating them with different concentrations of GFP to trap the antigen. Trapper-coated beads that where not incubated with GFP were used as a negative control. After incubation with the library, phage were eluted either with a GFP-specific stripper (CA12760 SEQ ID NO:1) or with trypsin. Two rounds of selection were performed. Output phage from each elution were recovered by infecting E. coli and the enrichment was evaluated by comparing serial dilutions of these cells according to Pardon et al., (2014).

FIGS. 3A and 3B. Competition binding analysis of GFP-specific Nanobodies by Bio-layer interferometry.

The binding properties of the newly discovered GFP-specific Nanobodies were analysed by a Bio-layer interferometry (BLI) on an OctetRed (molecular devices). Streptavidin-coated Octet® biosensors were used to capture biotinylated GFP (100 nM). Next, the picomolar GFP stripper (CA12760; SEQ ID NO:1) was allowed to bind to GFP until a plateau was reached. These CA12760-saturated biosensors were next incubated separately in solutions containing each newly discovered Nb (as indicated in the legend of the figure), supplemented with CA12760 Nb. Assays were performed in HEPES 25 mM pH 7.5, NaCl 150 mM supplemented with BSA 0.1% and Tween₂₀ 0.005% at room temperature. All Nanobodies (CA17517, CA17676, CA17518 in A; CA17674, CA17673, CA17519, CA17520, CA17675 in FIG. 3B) except the stripper (CA12760 in FIG. 3A) or the irrelevant Nb (CA8780 in FIG. 3A) cause an apparent mass increase on the sensor, indicating that they bind an epitope that does not overlap with the epitope of the stripper.

FIGS. 4A and 4B. Characterization of FBA1-specific Nanobodies selected from a proteome-wide antibody library by NANEX using a GFP-specific trapper/stripper pair.

Four FBA1-specific Nanobodies (40=Nb clone CA17440 corresponding to SEQ ID NO: 3, 41=CA17441-SEQ ID NO: 4, 42=CA17442-SEQ ID NO: 5, 43=CA17443-SEQ ID NO: 6) were characterized by co-immunoprecipitation assays. Each FBA1-specific Nanobody was covalently linked to NHS-agarose beads. The beads that were functionalized with these FBA1-specific Nanobodies were incubated for one hour at 4° C. on a rotating device with either an EBY100 lysate or the lysate of the engineered Yeast strain expressing FBA1 (Yeast GFP fusion collection reference: GFP(+)22, G1) as a GFP-tagged protein. After washing, these separate beads were resuspended in SDS-PAGE loading dye, boiled and analyzed on SDS-PAGE (FIG. 4A). Separated proteins were transferred to a PVDF membrane and the Western blot (FIG. 4B) was developed with a GFP-specific antibody to confirm the presence of GFP-tagged FBA1.

FIGS. 5A and 5B. Characterization of PDC1-specific Nanobodies selected from a proteome-wide antibody library by NANEX using a GFP-specific trapper/stripper pair.

Two PDC1-specific Nanobodies (51=Nb clone CA17451 corresponding to SEQ ID NO: 7, 52=CA17452-SEQ ID NO: 8) were characterized by co-immunoprecipitation assays. Each PDC1-specific Nanobodies was covalently linked to NHS-agarose beads. The beads that were functionalized with these PDC1-specific Nanobodies were incubated for an hour at 4° C. on a rotating device with either an EBY100 lysate or the lysate of the engineered Yeast strain expressing PDC1 (Yeast GFP fusion collection reference: GFP(+)12, F8) as a GFP-tagged protein. After washing, these beads were resuspended in SDS-PAGE loading dye and analyzed on SDS-PAGE (FIG. 5A). Separated proteins were transferred to a PVDF membrane and the Western blot (FIG. 5B) was developed with GFP-specific antibody to confirm the presence of GFP-tagged PDC1.

FIGS. 6A and 6B. Characterization of SSA1-specific Nanobodies selected from a proteome-wide antibody library by NANEX using a GFP-specific trapper/stripper pair.

Six SSA1-specific Nanobodies (60=Nb clone CA17560 corresponding to SEQ ID NO: 19, 61=CA17561-SEQ ID NO: 20, 62=CA17562-SEQ ID NO: 21, 63=CA17563-SEQ ID NO: 22, 64=CA17564-SEQ ID NO: 23, 65=CA17565-SEQ ID NO: 24) were characterized by co-immunoprecipitation assays. Each SSA1-specific Nanobodies was covalently linked to NHS-agarose beads. The beads that were functionalized with these SSA1-specific Nanobodies were incubated for an hour at 4° C. on a rotating device with either an EBY100 lysate or the lysate of the engineered Yeast strain expressing SSA1 (Yeast GFP fusion collection reference: GFP(+)10, E4) as a GFP-tagged protein. After washing, these beads were resuspended in SDS-PAGE loading dye and analyzed on SDS-PAGE (FIG. 6A). Separated proteins were transferred to a PVDF membrane and the Western blot (FIG. 6B) was developed with GFP-specific antibody to confirm the presence of GFP-tagged SSA1.

FIGS. 7A and 7B. Characterization of PGI1-specific Nanobodies selected from a proteome-wide antibody library by NANEX using a GFP-specific trapper/stripper pair.

Three PGI1-specific Nanobodies (55=Nb clone CA17455 corresponding to SEQ ID NO: 15, 56=CA17456-SEQ ID NO: 16, 57=CA17457-SEQ ID NO: 17) were characterized by co-immunoprecipitation assays. Each PGI1-specific Nanobodies was covalently linked to NHS-agarose beads. The beads that were functionalized with these PGI1-specific Nanobodies were incubated for an hour at 4° C. on a rotating device with either an EBY100 lysate or the lysate of the engineered Yeast strain expressing PGI1 (Yeast GFP fusion collection reference: GFP(+)12, H11) as a GFP-tagged protein. After washing, these beads were resuspended in SDS-PAGE loading dye and analyzed on SDS-PAGE (FIG. 7A). Separated proteins were transferred to a PVDF membrane and the Western blot (FIG. 7B) was developed with GFP-specific antibody to confirm the presence of GFP-tagged PGI1.

FIGS. 8A and 8B. Characterization of SIS1-specific, ALD6-specific, BMH1-specific Nanobodies selected from a proteome-wide antibody library by NANEX using a GFP-specific trapper/stripper pair.

One SIS1-specific Nanobody (44=Nb clone CA17444 corresponding to SEQ ID NO: 9), three ALD6-specific Nanobodies (53=CA17453-SEQ ID NO: 10, 54=CA17454-SEQ ID NO: 11, 60=CA17460-SEQ ID NO: 12), two BMH1-specific Nanobodies (58=CA17458-SEQ ID NO: 13, 59=CA17459-SEQ ID NO: 14) were characterized by co-immunoprecipitation assays. Each target-specific Nanobodies was covalently linked to NHS-agarose beads. The beads that were functionalized with these target-specific Nanobodies were incubated for an hour at 4° C. on a rotating device with either an EBY100 lysate or the lysate of the engineered Yeast strain expressing the target (Yeast GFP fusion collection reference for SIS1: GFP(+)22, E5, for ALD6: GFP(+)15, F1, for BMH1: GFP(+)27, D5) as a GFP-tagged protein. After washing, these beads were resuspended in SDS-PAGE loading dye and analyzed on SDS-PAGE (FIG. 8A). Separated proteins were transferred to a PVDF membrane and the Western blot (FIG. 8B) was developed with GFP-specific antibody to confirm the presence of GFP-tagged targets.

FIGS. 9A and 9B. Characterization of SXM1-specific Nanobodies selected from a proteome-wide antibody library by NANEX using a GFP-specific trapper/stripper pair.

One SXM1-specific Nanobody (30=Nb clone CA17530 corresponding to SEQ ID NO: 18) was characterized by co-immunoprecipitation assays. The SXM1-specific Nanobody was covalently linked to NHS-agarose beads. The beads that were functionalized with this SXM1-specific Nanobody were incubated for an hour at 4° C. on a rotating device with either an EBY100 lysate or the lysate of the engineered Yeast strain expressing SXM1 GFP(+)04, H9) as a GFP-tagged protein. After washing, these beads were resuspended in SDS-PAGE loading dye and analyzed on SDS-PAGE (FIG. 9A). Separated proteins were transferred to a PVDF membrane and the Western blot (FIG. 9B) was developed with GFP-specific antibody to confirm the presence of GFP-tagged SXM1.

FIG. 10. NANEX to capture and immobilize PGI1 binders from the soluble fraction of a yeast lysate.

Beads were functionalized with the PGI1-specific Nanobody CA17455 clone (SEQ ID NO:15) to be used as a trapper. CA17455-functionalized beads were next incubated for one hour at 4° C. on a rotating device with an EBY100 lysate and washed. Next, these beads were incubated for 1 h with the same PGI1-specific Nanobody to elute the target (PGI1) in association with several interacting proteins (LYS20 UniProt P48570, TDH3 UniProt P00359, PNC1 UniProt P53184), as shown by mass spectrometry analysis.

FIG. 11. Characterization of GR-LBD-specific Nanobodies selected from a GR-LBD antibody library by NANEX using a GFP-specific trapper/stripper pair.

Five GR-LBD-specific Nanobodies (97=Nb clone CA17797 corresponding to SEQ ID NO: 35, 98=CA17798-SEQ ID NO: 36, 99=CA17799-SEQ ID NO: 37, 00=CA17800-SEQ ID NO: 38, 01=CA17801-SEQ ID NO: 39) were characterized by co-immunoprecipitation assays. Each GR-LBD-specific Nanobody was covalently linked to NHS-agarose beads. The beads that were functionalized with these GR-LBD-specific

Nanobodies were incubated for one hour at 4° C. on a rotating device with either a HEK293T lysate (supplemented with DEX or not) transfected with a PCDNA3.1 plasmid containing the GFP-GR (full-length) gene expressing full-length GR as a GFP-tagged protein. After washing, these separate beads were resuspended in SDS-PAGE loading dye, boiled and analyzed on SDS-PAGE (not shown). Separated proteins were transferred to a PVDF membrane and the Western blot was developed with a GR-specific antibody to confirm the presence of GFP-tagged GR. NC stands for negative control Nb. GFP=27 kDa; GFP-GR fusion=117 kDa; GR (cleaved)=90 kDa.

FIGS. 12A and 12B. Enrichment after the first and second round of selection on 94 different GFP-POIs using NANEX.

NANEX was used for the selection of POI-specific antibodies from a proteome-wide antibody display library according to example 3. A GFP trapper (CA15816 corresponding to SEQ ID NO:2) was coupled to magnetic beads and dispensed in 96 different wells. NANEX beads were incubated with different lysates (table 3) of engineered Yeasts expressing GFP-POIs. Following several washing steps, phage were added to the wells. After incubation with the library, phage were eluted with a GFP-specific stripper (CA12760 corresponding to SEQ ID NO:1). Two rounds of selection were performed (R1 in FIG. 12A and R2 in FIG. 12B). Output phage from each elution were recovered by infecting E. coli and the enrichment was evaluated by comparing serial dilutions of these cells according to Pardon et al. (2014). Data are present for each well as listed chronologically in Table 3 (A1-F12 for each of the 94 GFP-targets from left to right on X-axis G12 and H12 as last samples on X-axis for controls)).

FIG. 13. Characterization of POI-specific Nanobodies selected from a proteome-wide antibody library by NANEX using a GFP-specific trapper/stripper pair.

One HSP104-specific Nanobody (04=CA18504 SEQ ID NO:40), one MET6-specific Nanobody (05=CA18505 SEQ ID NO:41), two SBA1-specific Nanobodies (08=Nb clone CA18508 corresponding to SEQ ID NO:42, 09=CA18509-SEQ ID NO:43), one SOD1-specific Nanobody (10=CA18510-SEQ ID NO:44), and a ENO1-specific Nanobody (38=CA17938-SEQ ID NO:45), were characterized by co-immunoprecipitation assays. Each POI-specific Nanobodies was covalently linked to NHS-agarose beads. The beads that were functionalized with these POI-specific Nanobodies were incubated for an hour at 4° C. on a rotating device with the lysate of the engineered Yeast strain expressing the POIs (Yeast GFP fusion collection reference: GFP(+)05, A2 (HSP104); GFP(+)07, D4 (MET6); GFP(+)30, A6 (SBA1); GFP(+)33, F8 (SOD1) and GFP(+)17, D12 (ENO1) as a GFP-tagged protein. After washing, these beads were analyzed on SDS-PAGE (not shown) and Western blot was developed with GFP-specific antibody to confirm the presence of GFP-tagged POIs.

FIGS. 14A and 14B. Characterization of PGI1-specific Nanobodies selected from a proteome-wide antibody library by NANEX using a PGI1-specific trapper/stripper pair.

Six PGI1-specific Nanobodies (91=Nb clone CA17791 corresponding to SEQ ID NO: 46, 92=CA17792-SEQ ID NO: 47, 93=CA17793-SEQ ID NO: 48, 94=CA17794-SEQ ID NO: 49, 95=CA17795-SEQ ID NO: 50, 96=CA17796-SEQ ID NO: 51) were characterized by co-immunoprecipitation assays. Each PGI1-specific Nanobody was covalently linked to NHS-agarose beads. The beads that were functionalized with these PGI1-specific Nanobodies were incubated for an hour at 4° C. on a rotating device with either an EBY100 lysate or the lysate of the engineered Yeast strain expressing PGI1 (Yeast GFP fusion collection reference: GFP(+)12, H11) as a GFP-tagged protein. After washing, these beads analyzed on SDS-PAGE (FIG. 14A). Separated proteins were transferred to a PVDF membrane and the Western blot was developed with a GFP-specific antibody to confirm the presence of GFP-tagged PGI1 (FIG. 14B). 55=Nb clone CA17455 corresponding to SEQ ID NO: 15, used as trapper/stripper for PGI1, serves as a positive control.

FIG. 15. Characterization of rVGLUT1-specific Nanobodies selected from a rVGLUT1 antibody library by NANEX using a rVGLUT1-specific trapper/stripper pair.

A rVLGUT1-specific Nanobody (25=Nb clone CA18425 corresponding to SEQ ID NO: 53) was characterized by co-immunoprecipitation assays. The rVGLUT1-specific Nanobody was covalently linked to NHS-agarose beads. The beads that were functionalized with this rVGLUT1-specific Nanobody were incubated for one hour at 4° C. on a rotating device with a HEK293T lysate transfected with a plasmid expressing full-length rVGLUT1 as a cMyc-YFP-tagged protein. After washing, the beads were resuspended in SDS-PAGE loading dye, without boiling and analyzed on SDS-PAGE (not shown). Separated proteins were transferred to a PVDF membrane and the Western blot was developed with a c-Myc-specific antibody to confirm the presence of c-Myc-tagged rVGLUT1. NC stands for negative control Nb.

FIG. 16. Characterization of rVGLUT1-specific Nanobodies selected from a synaptic proteome antibody library by NANEX using a GFP-specific trapper/stripper pair.

Six rVLGUT1-YFP specific Nanobodies (24=Nb clone CA18024 corresponding to SEQ ID NO:54, 37=CA18437-SEQ ID NO:55, 38=CA18438-SEQ ID NO: 56, 39=CA18439-SEQ ID NO:57, 40=CA18440-SEQ ID NO:58, 41=CA18441-SEQ ID NO:59) were characterized by co-immunoprecipitation assays. Each rVGLUT1-YFP specific Nanobody was covalently linked to NHS-agarose beads. The beads that were functionalized with these rVGLUT1-specific Nanobodies were incubated for one hour at 4° C. on a rotating device with a HEK293T lysate transfected with a plasmid containing a gene expressing full-length rVGLUT1 as a c-Myc-YFP-tagged protein. After washing, the beads were resuspended in SDS-PAGE loading dye, without boiling and analyzed on SDS-PAGE (not shown). Separated proteins were transferred to a PVDF membrane and the Western blot was developed with a c-Myc-specific antibody to confirm the presence of c-Myc-tagged rVGLUT1. NC stands for negative control Nb.

FIG. 17. Characterization of GR-specific Nanobodies selected from an GFP-GR antibody library by NANEX using a mCherry-specific trapper/stripper pair.

Seven GR-specific Nanobodies (98=Nb clone CA18498 corresponding to SEQ ID NO: 62, 99=CA18499-SEQ ID NO: 63, 01=CA18501-SEQ ID NO: 64, 02=CA18502-SEQ ID NO: 65, 03=CA18503-SEQ ID NO: 66, 85=CA18585-SEQ ID NO: 67, 86=CA18586-SEQ ID NO: 68) were characterized by co-immunoprecipitation assays. Each GR-specific Nanobody was covalently linked to NHS-agarose beads. The beads that were functionalized with these GR-specific Nanobodies were incubated for one hour at 4° C. on a rotating device with a HEK293T lysate transfected with a pcDNA3.1 plasmid containing the mCherry-GR (full-length) gene expressing full-length GR as a mCherry-tagged protein. After washing, these separate beads were resuspended in SDS-PAGE loading dye, boiled and analyzed on SDS-PAGE (not shown). Separated proteins were transferred to PVDF membrane and the Western blot was developed with a GR-specific antibody to confirm the presence of GR. NC stands for negative control Nb.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions. By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., a polypeptide binder, or the target protein as identified and disclosed herein which has been removed from the molecules present in a sample or mixture, such as a production host, that are adjacent to said polypeptide. An isolated protein or peptide can be generated by amino acid chemical synthesis or can be generated by recombinant production or by purification from a complex sample.

The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link, such as a heterologous tag that is covalently linked to a target protein.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant”, “engineered” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product.

The term “binding site” refers to a region of a molecule or molecular complex, that, as a result of its shape and charge, favourably associates with another chemical entity, compound, proteins, peptide, antibody or Nb. For antibody-related molecules, the term “epitope” or “conformational epitope” is also used interchangeably herein. The term “pocket” includes, but is not limited to cleft, channel or site. The term “part of a binding pocket/site” or “partially overlapping epitope” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in target protein binding, or may be residues that are spatially related and define a three-dimensional compartment of the binding pocket, or that confer a conformational function. An ‘adjacent’ or ‘minimally overlapping’ binding site, as used herein, refers to ‘no overlapping amino acids (but binding to a site close by)’, or maximum of about 30% overlap in the binding amino acid residues respectively. An “epitope”, as used herein, refers to an antigenic determinant of a polypeptide, constituting a binding site or binding pocket on a target molecule. Said epitopes on the target protein may be comprise at least one amino acid that is essential for binding the binding agent, though preferably comprise at least 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography, multi-dimensional nuclear magnetic resonance, Cryo-EM, Hydrogen Deuterium-Exchange (HDX)-MS, as well as Cross-linking Mass-spectrometry (XL-MS), epitope binning, or used to a lower extent also Neutron scattering, X-ray Free electron-laser (XFEL) or Small-angle neutron scattering (SANS) and small-angle x-ray scattering (SAXS) technology. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. Affinity is typically measured and reported by the equilibrium dissociation constant (K_{D}), which is used to evaluate and rank order strengths of bimolecular interactions. The binding of an antibody to its antigen is a reversible process, and the rate of the binding reaction is proportional to the concentrations of the reactants. At equilibrium, the rate of [antibody] [antigen] complex formation is equal to the rate of dissociation into its components [antibody]+[antigen]. The measurement of the reaction rate constants can be used to define an equilibrium or affinity constant (1/K_{D}). In short, the smaller the K_{D} value the greater the affinity of the antibody for its target. The rate constants of both directions of the reaction are termed: the association reaction rate constant (kₒₙ), which is the part of the reaction used to calculate the “on-rate” (kₒₙ), a constant used to characterize how quickly the antibody binds to its target. Vice versa, the dissociation reaction rate constant (k_{off}), is the part of the reaction used to calculate the “off-rate” (k_{off}), a constant used to characterize how quickly an antibody dissociates from its target. In measurements as shown herein, the flatter the slope, the slower off-rate, or the stronger antibody binding. Vice versa, the steeper downside indicates a faster off-rate and weaker antibody binding. The ratio of the experimentally measured off- and on-rates (k_{off}/kₒₙ) is used to calculate the K_{D} value. Several determination methods are known to the skilled person to measure on and off rates and to thereof calculate the K_{D}, which is therefore, taking into account standard errors, considered as a value that is independent of the assay used.

A “binding agent”, or “agent” as used interchangeably herein, relates to a molecule that is capable of binding to another molecule, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others. Binding may be obtained through a covalent or non-covalent linkage.

The term “antibody” refers to an immunoglobulin (Ig) molecule or a molecule comprising an immunoglobulin (Ig) domain, which specifically binds with an antigen. ‘Antibodies’ can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains (ISVDs), Nanobodies (or VHH antibodies), domain antibodies, and single chain structures, such as a complete light chain or complete heavy chain.

The term “antibody fragment” and “active antibody fragment” or “functional variant” as used herein refer to a protein comprising an immunoglobulin domain or an antigen-binding domain comprising the required CDRs and/or structural features to specifically bind a target protein. Antibodies are typically tetramers of immunoglobulin molecules. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al. (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. For numbering of the amino acid residues of an IVD different numbering schemes can be applied. For example, numbering can be performed according to the AHo numbering scheme for all heavy (VH) and light chain variable domains (VL) given by Honegger, A. and Plückthun, A. (J. Mol. Biol. 309, 2001), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids in the article of Riechmann, L. and Muyldermans, S., 231(1-2), J Immunol Methods. 1999. It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein. Determination of CDR regions may also be done according to different methods, such as the designation based on contact analysis and binding site topography as described in MacCallum et al. (J. Mol. Biol. (1996) 262, 732-745). Or alternatively the annotation of CDRs may be done according to AbM (AbM is Oxford Molecular Ltd.'s antibody modelling package as described on http://www.bioinf.org.uk/abs/index.html), Chothia (Chothia and Lesk, 1987; Mol Biol. 196:901-17), Kabat (Kabat et al., 1991; 5^{th} edition, NIH publication 91-3242), IMGT (LeFranc, 2014; Frontiers in Immunology. 5 (22): 1-22), and/or alternative annotations including aHo, Gelfand, and Honegger; see, e.g., Dondelinger et al. 2018, Front Immunol 9:2278 for a review). Said annotations further include delineation of CDRs and framework regions (FRs) in immunoglobulin-domain-containing proteins, and are known methods and systems to a skilled artisan who thus can apply these annotations onto any immunoglobulin protein sequences without undue burden. These annotations differ slightly, but each intend to comprise the regions of the loops involved in binding the target. When CDRs is referred to herein, at least one of those above annotations is applicable, preferably the IMGT annotation.

VHHs or Nbs are often classified in different sequence families or even superfamilies, as to cluster the clonally related sequences derived from the same progenitor during B cell maturation (Deschaght et al. 2017. Front Immunol. 10; 8:420). This classification is often based on the CDR sequence of the Nbs, and wherein for instance each Nb family is defined as a cluster of (clonally) related sequences with a sequence identity threshold of the CDR3 region. Within a single VHH family defined herein, the CDR3 sequence is thus identical or very similar in amino acid composition, preferably with an identical length and at least 80% identity, or at least 85% identity, or at least 90% identity in the CDR3 sequence, resulting in Nbs of the same family binding to the same binding site, having the same effect or functional impact.

As used herein, the terms “determining,” “measuring,” “assessing,”, “identifying”, “screening”, and “assaying” are used interchangeably and include both quantitative and qualitative determinations.

### DETAILED DESCRIPTION

The present invention relates to a novel selection approach for identifying specific polypeptide binders for a protein of interest from a plurality of binders, specifically from a library of protein binders, wherein the NANEX-based affinity displacement method is integrated in first instance to isolate and immobilize the target protein through binding to a first protein binding agent that is coupled to a surface, and secondly by making use of the affinity displacement principle concept of NANEX for elution of the polypeptide binder in complex with the target protein. This integrated selection method provides for a medium- to high-throughput approach in recombinant antibody library screening and provides for a high selectivity complementary to the power of display technology. This novel method is first in its approach in the field of recombinant antibody library selection and panning, and has several advantages inherent to the NANEX-based system, as explained here below clarifying Nanobody Exchange Chromatography (NANEX) as originally established. Moreover, this novel approach provides for several benefits and improvements over (conventional) in vitro selection methods using phage-display, wherein selection involves exposure to immobilized antigen to allow antigen-specific phage antibodies to bind their targets during bio-panning, followed by the recovery of antigen-bound phage and subsequent infection in bacteria. Most in vitro selection methods depend on purified antigens and require harsh conditions (high salt, extreme pH, proteolysis) to elute the phage from the immobilized targets. In contrast, NANEX-based selection allows us to trap and immobilize complex antigens from native samples, next apply phage libraries and subsequently strip the antigen-bound phage in a target specific manner, all this under fully native conditions. NANEX also allows us to distinctively select for Nbs that bind epitopes that do not overlap with the trapper-stripper pair.

### NANEX Affinity Displacement

The Nanobody exchange chromatography method (referred to herein as ‘NANEX’, based on the previously described method in PCT/EP2020/087291), relates to the purification of proteins by affinity displacement chromatography, making use of small antigen-binding entities, thereby establishing a favorable kinetic context. In particular, a pair of target-specific protein binding agents specifically binding an epitope on a target in a competitive manner is used in a complementary kinetic context. A pair of binders for the same target may involve competing or non-overlapping binding site, or rather different binding sites at non-overlapping or different epitopes. Typically, affinity displacement acts via transient sandwich complexes through pairs of binders, within defined dose—and kinetic relations. However, in cases where a pair with the same or overlapping epitope is used, binding kinetics and the nature of the binding agent affect the balance between a cross-block or displacement. In NANEX, it was observed that when using antigen-binding domains built on immunoglobulin single variable domains (ISVDs), or more specifically Nbs, as displacers, targeting the same or largely overlapping and thus competing epitope as the trapper on a target protein is most efficiently established if the dissociation rate constant (k_{off}) is lower for the second than the first protein binding agent. Furthermore, purely based on their competitive nature, one could use the same binding agent, such as a Nanobody for binding (or trapping) and eluting (or stripping) to purify the target, to obtain a (suboptimal) satisfying yield of purified protein in the elution fraction. When searching for a binding agent that is capable of fully outcompeting the trapper binding to the target, which is desired in high-throughput applications such as the selection method described herein, one would find that the use of conventional antibody binders (such as monoclonal antibodies) to the same epitope mostly block any displacement reaction and one would rather use protein binding agents such as antibodies binding to an adjacent or minimally overlapping epitope as compared to the trapper, as to avoid such a block of the epitope by competition. However, by using NANEX, which provides for at least the stripper second protein binding agent an ISVD, binding the same, substantially the same, or large overlapping epitope as the first protein binding agent, and wherein said second ISVD-comprising protein binding agent has a lower dissociation rate constant (k_{off}), a very efficient displacement of the first protein binding agent is obtained, allowing elution of the target protein at high yields and with high specificity. So the compact but highly specific nature of the ISVD-type of antigen binders provides for a kinetic relation in competing binding modes to efficiently displace another antigen-binding protein, which is advantageous over the larger conventional antibodies with a larger antigen-binding site/paratope (composed of residues from 6 CDRs instead of 3 CDRs for the ISVDs). In addition, this reaction may take place in mild physiological condition, and still providing efficient elution, which is therefore perfectly suitable for integration in the selection method described herein, since polypeptide binders from the plurality of binders that are bound to the target protein can remain in complex with the target protein, and be co-eluted with the stripper-target complex. So by integrating NANEX into this novel selection approach, the combined NANEX-purification and selection-conditions established herein resulted in a next-generation improved selection for polypeptide binders, specifically in antibody drug discovery. Indeed, the results as exemplified herein have clearly shown that the integrated NANEX-selection procedure provides for robust and reliable selection of displayed polypeptide binders in a more efficient and in-depth manner as compared to conventional selection panning methods.

The ‘ISVD-based displacement’, or more particularly ‘Nanobody exchange’ or ‘Nanobody exchange chromatography’ or ‘NANEX’, as interchangeably used herein, which previously focused on analytical purification for straightforward highly pure protein complex elution, now finds its application in antibody drug discovery and target-binder identification approaches. The strength of this novel selection method is thus in the use of NANEX for presenting the target, since the Nb-based trapper/stripper compete for the same or highly overlapping epitope and provide for high affinity binders that do not disturb other binders at different epitope. This allows not only to find binders for new or different epitopes, but also to find binders for certain conformations of the target (when trapper/stripper is used that locks the target in a particular conformation).

When using NANEX purification, the elution complex thus contains the stripper or displacer, which has the advantage that this allows to apply ISVD-comprising second protein binding agents (called strippers, or Nanostripper in the case of Nanobodies) that are additionally functionalized, i.e. they provide for a specific function to the eluted protein complex. Such a functionalization may relate to visualization of the protein complex (via fluorescence or labelling of the agent) or relates to functioning as a chaperone or adapter protein (including for instance but not limited to a MegaBody), among other examples, to elute the target in a functionalized complex (see also below). Moreover, following the elution step and a regeneration procedure, the affinity matrix, which may be any type of surface, such as (magnetic) beads, a column, a well in a plate, or a resin, is ready for the next affinity purification and/or selection cycle and can be used in high-throughput platforms, such as a screening platform, a chip, or a microfluidics setup or device.

By using the NANEX or Nanobody exchange chromatography-integrated selection method as described herein, a leap forward can be foreseen in high-throughput selection of binders for several target classes such as intractable proteins or difficult to purify protein. Moreover, selection of protein binding agents with conformation-selective recognition of antigens or targets, fixed in a certain conformational state by the trapper, such as a stabilized conformation, an active/inactive conformation, more specifically an agonist, partial agonist or biased agonist conformation can be selected for. Alternatively, when NANEX-trapper/strippers for a heterologous tag, such as GFP, are set forth in this selection method described herein, a generic high-throughput selection platform can be developed, providing further benefits to automated and high efficient antibody screening technology. With the rapid advancement of such technologies in biotechnology, it is foreseeable that the invention will impact the efficiency and potential of novel therapeutic drug screening as well as increase throughput and the potential of proteomics, MS-based, structural and other analytics.

### NANEX-Based Selection of Polypeptide Binders

The method for selection of a polypeptide binder specific for a target protein thus relates in a first aspect to a method comprising the steps of:
a) mixing a first protein binding agent that is immobilized on a surface and specifically binding a target protein with a sample comprising the target protein, for obtaining a complex on the surface,
b) providing to said complex of step a) a sample comprising a plurality of polypeptide binders,
c) adding to the mixture of step b) a sample comprising a second protein binding agent, which competes with the first binding agent for binding to said target protein, and which by specifically binding the target protein displaces the first binding agent from the target protein, and
d) eluting the second protein binding agent bound to the target protein, for isolation of a polypeptide binder bound to said target protein.

The feature as to ‘compete for the target protein binding’ required for efficient elution of the target-stripper complex in association with a polypeptide binder may be interpreted as a stripper competing for the same epitope, or may also mean competing in a different manner, such a kinetically or allosterically, since especially in the field of Nbs, allosteric interactions with a target are known to represent the binding mode. So in one embodiment, the stripper may compete for binding the target by binding to minimally overlapping or adjacent epitopes, or alternatively, the stripper can even disrupt the interaction between the trapper and the target by binding to an allosteric site on the target, by inducing a conformational change of the target. Competing binding agents may be established using several methods as known in the art, for example, but not limited to, a competition ELISA, alphalisa, Octet measurements or bio-layer interferometry (BLI), SPR Biacore, Microscale thermophoresis (MST), amongst others.

So the competitive elution mode is used for elution of the target from its immobilized surface, and thus not involve any competition with regards to the selected polypeptide binder (which is ideally also bound to the target at the stage of elution). Though, the use of at least an ISVD as antigen-binding second protein binding agent or stripper is desired in this competitive elution as used in NANEX since the use of ISVDs or Nbs provides for more favorable kinetics as compared to for instance the use of large conventional antibodies as displacers.

The method of the present invention comprises a second protein binding agent, which is in solution, and soluble in elution conditions. Said elution conditions preferably relate to physiological conditions, as known to the skilled person. The term ‘soluble’ as used herein refers to the fact that the protein binding agent is in a functional form, meaning that it is capable of specifically binding its target within the expected range of its affinity for the epitope. Said first protein binding agent is immobilized for the method of the present invention in step a), wherein it may be immobilized on a surface via covalent or other means of coupling. The agent may be a coupled to beads, which may be agarose or magnetic beads, or may be present on a surface or matrix, more specifically on packed as an affinity column, which may be suited for preparative as well as analytical scales, more particularly, which may be a microcolumn in the order of below 1 mL column volume, or even in submicromolar volumes, or even provided on a chip using microfluidics technology. Most preferably, said first protein binding agent is immobilized on a solid support or resin. A ‘resin’ or ‘affinity resin’ as used interchangeably herein, is an activated affinity chromatography support for the immobilization of biomolecules such as ISVDs or other protein binding agents. In a specific embodiment, said first protein binding agent comprises an ISVD and is coupled to a resin using known coupling methods from the art (see examples). The method as presented herein has the advantage that upon elution in step d), using physiological conditions. Optionally, depending on the type of sample used in a) and the duration of the selection, a mild regeneration step of the immobilized surface may allow to reuse said immobilized complex in a second round, though preferably, further rounds of selection include a repetition of the method steps a) to d) so the integrity and stability of the target protein is assured.

Depending on the nature of the sample, an optional washing of the immobilized surface after step a) or step b) of the method of the present invention may be required, under neutral, or very mild, or rather harsh conditions, or may require repetitions of washing steps to remove any unbound abundant components that were present in the sample comprising the target protein, or the remove the unbound remainders of the plurality of polypeptide binders.

In a further embodiment, said method as described herein comprises a second protein binding agent with a k_{off} that is lower as compared to the k_{off} of the first protein binding agent. Further, said method delivers the most optimal result when the first binding agent or trapper has a higher dissociation rate or lower or equal affinity as compared to the second binding agent, and vice versa, when the second binding agent has a lower dissociation rate and/or the same or higher affinity for the epitope as compared to the first binding agent. From current state of the art knowledge, the rate constant of dissociation (or off-rate or k_{off}) and the rate constant of association (or on-rate or kₒₙ) are interrelated as K_{D}=K_{off}/kₒₙ, wherein K_{D} is defined as the dissociation constant, which is inversely correlated with the affinity of a binding agent for its target, as described also in detail in the definitions above. So, if the dissociation constant K_{D} value is low(er), the affinity is high(er) (if kₒₙ is the same). Alternatively, if kₒₙ is higher, the K_{D} is lower and the affinity is higher (if k_{off} is the same). So, for the selection method of the present invention, the protein binding agents described herein are relatively different in k_{off} and/or affinity (or K_{D}) for the same, substantially the same or largely overlapping epitope. The method as described herein refers more specifically to the k_{off} of the second binding agent being lower than the K_{off} of the first binding agent for the same, substantially same, or largely overlapping epitope of the target protein, with ‘lower’ referring herein to a value that is at least 2-fold lower, 5-fold lower, or 10-fold lower, or at least 30-fold lower, or at least 100-fold lower, or at least 200-fold, at least 300-fold, at least 400-fold, or at least 500-fold lower. More preferably, said k_{off} value of the second binding agent is in the range of at least 2-fold lower to at least 10-fold lower, or at least 5-fold lower to at least 20-fold lower, or at least 10-fold lower to at least 30-fold lower, or at least 100-fold lower, as compared to the K_{off} value of the first protein binding agent.

Similarly, the affinity of the second binding agent may be equal or higher than the affinity of the first binding agent for the epitope of the target protein, wherein ‘higher affinity’ refers to a ‘K_{D} value’ of the second protein binding agent being a K_{D} value that is at least 2-fold lower, or at least 5-fold lower, 10-fold lower, 20-fold lower or 100-fold lower, or in the range of at least 2- to at least 2000-fold lower, as compared to the K_{D} value of the first protein binding agent. In a preferred embodiment, the purification method as described herein discloses a first binding agent with a K_{D} value for the epitope of the target protein of 1 mM to about 1 nanomolar and discloses a second protein binding agent with a K_{D} value, optionally with substantially the same or largely overlapping epitope for said target, of 1 nanomolar or lower, optionally down to 1 picomolar. More preferably, said first binding agent has a K_{D} in the nano- to millimolar range (i.e. 10E-9 to 10E-3) and the second binding agent has a K_{D} value in the femto- to micromolar range (i.e. 10E-12 to 10E-6), most preferable with a relative difference between the first and second binding agent of at least 2-fold. In one embodiment said K_{D} value for the first protein binding agent is at least 2-fold higher than the K_{D} of the displacer, a difference which is driven by the difference in k_{off} value, especially when the displacer binds to the same or largely overlapping epitope.

### A Plurality of Polypeptide Binders

The method as described herein selects from a sample comprising a “plurality of polypeptide or proteinaceous binders”, which may in fact be any type of proteins or peptides or polypeptides, such as in the broadest sense a collection of proteins acting as candidates to specifically bind the target protein, wherein said ‘plurality of polypeptide binders’ in the broadest sense may thus for instance be derived from a cell-extract, a specific tissue or signal transduction cascade, or unbiased proteome samples. In a more specific aspect, the ‘plurality of protein binders’ is provided as a sample comprising a repertoire of binders as fragments expressed and/or displayed from a library. More specifically display libraries are mostly known for antibody type of molecules. Recombinant antibody libraries are thus in scope of such a sample with a plurality of protein binders, as such antibody libraries will often provide for protein binders selected to specifically bind the target protein.

The ‘plurality of polypeptide binders’ may thus be structurally provided by binding agents comprising a binding domain (potentially binding the target protein), and provided by a protein, a peptide, or a peptidomimetic, and more specifically an ‘antigen-binding’ domain, as present in many different antibody and antibody-like molecules, as described herein, for instance but not limited to antibodies or active antibody fragments, such as Fab, Fab′ and F(ab′)2, Fd, single-chain Fvs (scFv), single-chain antibodies, disulfide-linked Fvs (dsFv) and fragments comprising either a VL or VH domain, a heavy chain antibody (hcAb), a single domain antibody (sdAb), a minibody, the variable domain derived from camelid heavy chain antibodies (VHH or Nanobody), the variable domain of the new antigen receptors derived from shark antibodies (VNAR), a protein scaffold including an Alphabody, designed ankyrin-repeat domains (DARPins), fibronectin type III repeats, anticalins, knottins, engineered CH2 domains (nanoantibodies), among others.

In a preferred embodiment said ‘plurality of polypeptide binders’ is thus provided by a sample comprising a display library, allowing to select for a phenotype-to-genotype coupling, as described herein, which is key to facilitate the selection of antigen-specific antibodies or binders (antigen-binding domain containing polypeptides), a critical feature of antibody display technologies used for identification of therapeutic hits. So the term “display libraries” as used herein provides for those recombinant libraries that allow to physically link the phenotype (antigen-binding behaviour) to the genotype of a repertoire of polypeptide binders.

Moreover, the types of recombinant antibody libraries intended herein includes mAb libraries based on immune fragments (that is, biased towards certain specificities present in immunized animals or naturally immunized, or infected, humans) or naive fragments (not biased toward specificities found in the immune system). The latter type of fragment can be derived from nonimmune natural or semi-synthetic sources. Nonimmune (or naive) libraries are derived from natural, unimmunized, rearranged V genes (e.g., from the IgM B-cell pool) to reduce antigen-induced biases in the repertoire, and were the first libraries used to isolate anti-self antibodies—otherwise difficult to obtain by immunization. Synthetic antibody libraries are constructed entirely in vitro using oligonucleotides that introduce areas of complete or tailored degeneracy into the CDRs of one or more V genes.

As known in the art, several display technologies allow for different approaches in selection of recombinant antibody libraries, wherein phage display is the dominating technology, though alternatives including yeast, ribosome, bacteria, and mammalian display are also envisaged in the method as described herein.

In a specific embodiment, the method of selection as described herein is used for multiple selection rounds, in a reiterative process as to enrich for polypeptide binders present in the library, such as for the phage display libraries used in the method shown herein. Specifically when the collected elution contains the phage displaying the target-specific Nb, bound to the target-stripper complex, the phage solution for reinfecting E. coli is provided by the collected elution, as to provide the phage for a following round.

### Target Protein Sample and Trapper/Stripper Pairs in NANEX-Based Selection

As previously indicated, the target protein of the method described herein provides an epitope for the first and second protein binding agents (trapper and stripper) applied in steps a) and d) of the method, wherein said epitope may be a native, naturally occurring, and/or an endogenously available epitope on said target protein. When trapper and stripper recognize the native or endogenous protein, as present in the sample of step a), this will allow to capture the target from the sample to obtain the immobilized antigen-trapper complex. Another option is provided by having the epitope presented on a recombinantly produced target protein, provided as a purified or partially purified sample, not requiring a tag per se. For protein binding agent pairs (trapper/stripper) specifically binding a non-tagged target protein, in order to compete for the same target, one may screen and select to provide for a pair of competing binding agents, or one may design towards a higher affinity and lower affinity pair of protein binding agents. Indeed, using a 3D-structure of the stripper or 2nd protein binding agent bound to the target allows to design mutations in the binding site of the protein binding agent that will result in a reduced affinity or higher k_{off}, and thereby provide for compatible trapper or 1st protein binding agent. Besides, more straightforward methods, not requiring structural information also allow to determine pairs based on a single binding agent, once the sequence is known. As shown in the examples, in a non-limiting way, for GFP, when used as a ‘target protein’, based on a screening for different binders, their competing nature was analysed by epitope mapping using BLI, or alternatively, based on the sequence of a nanomolar binder, or stripper, using an alanine mutation scan in the CDR3 region may also be performed, known to be most critical in defining the binding kinetics, new pairs with lower dissociation rate constants can be identified, to function as trappers in the NANEX method. In this way, pairs of protein binding agents binding the same epitope with a different k_{off} or affinity will be obtained simply by introducing single or multiple mutations.

In an alternative embodiment relating to a method wherein the trapper and stripper comprise an ISVD, the ‘monovalent’ format may be used as a trapper, and a ‘multivalent’ format as a stripper, since multivalent formats have a higher avidity as compared to the monovalent forms, with higher k_{off}, resulting in more optimal elution yields and target protein purity as well. The term ‘monovalent format’ herein refers to an ISVD, as used herein, that can only recognize one antigenic determinant, while the term ‘multivalent’ format refers to an ISVD as used herein that can recognize more than one antigenic determinant, such as—but not limited to—bivalent, trivalent or tetravalent formats. Moreover, instead of a multivalent stripper, also a multiparatopic or multispecific stripper may be envisaged, wherein said stripper may comprise an identical building block binding to the same antigenic determinant, and at least one or more building blocks binding that may be different and may bind the same or another epitope on the target protein, or alternatively, an epitope on another target protein in complex with the first target protein.

In a further embodiment, the method as described herein makes use of a first and second protein binding agent (trapper and stripper), wherein at least one of those binding agents comprises an antigen-binding domain, as defined herein, or more specifically comprising at least one antibody, ISVD, VHH, Nanobody, or antigen-binding chimeric protein, defined herein as an ISVD fused to a scaffold protein via at least two sites, and preferably the scaffold protein domain comprising HopQ, YgjK or a derivative or variant thereof. The latter definition of said antigen-binding chimeric proteins is in fact also called a MegaBody, which may thus be applied in the method of the present invention as a first and/or second protein binding agent. The term MegaBody as used herein refers to the novel fusion proteins disclosed in Steyaert et al. (WO2019/086548A1), referring to the fusion protein comprising an antigen-binding domain, which is connected to a scaffold protein, wherein said scaffold protein is coupled to said antigen-binding domain at one or more amino acid sites accessible or exposed at the surface of said domain, resulting in an interruption of the topology of said antigen-binding domain. Said antigen-binding chimeric protein is further characterized in that it retains its antigen-binding functionality as compared to the antigen-binding domain not fused to said scaffold protein. The MegaBody as described herein relates to the particular MegaBody or antigen-binding chimeric protein for which the antigen-binding domain comprises an immunoglobulin single variable domain (ISVD) or a Nanobody, which is fused or connected to a scaffold protein, at an accessible surface of said ISVD domain (B turn or loop, excluding the CDRs), resulting in an interruption of the topology of said antigen-binding domain, and retaining its antigen-binding functionality, i.e. the specific epitope recognition. In a specific embodiment, said second protein binding agent relates to the MegaBody or antigen-binding chimeric protein comprising an ISVD connected to the scaffold protein via an insertion of the scaffold protein in the first beta-turn connecting the beta-strand A and B of the ISVD (as defined according to IMGT nomenclature, and as defined in WO2019/086548A1). In an even more specific embodiment, the scaffold protein used herein is the HopQ or YgjK scaffold protein, wherein the fusion of the scaffold interrupts the topology of the ISVD, but not its overall 3D-structure, neither its epitope-binding specificity. The ‘HopQ’ or ‘HopQ-derived’ scaffold as used herein relates to a protein scaffold of the Adhesin domain of the type 1 HopQ of Helicobacter pylori strain G27 (Protein Database: PDB 5LP2), or a circularly permutated protein thereof, also called cHopQ or c7HopQ (see also WO2019/086548A1). The ‘YgjkK or ‘YgjK-derived’ scaffold as used herein relates to a protein scaffold of the Escherichia coli K12 YgjK (PDB 3W7S), or a circularly permutated gene encoding said protein thereof, also called cYgjK (see also WO2019/086548A1).

Another embodiment relates to the method for selection, wherein the sample comprising a target protein in step a) as described herein, provides for a target protein with an epitope recognized by the first and second protein binding agent that is present on the scaffold protein in a MegaBody (target protein). Said MegaBody may preferably be built using a scaffold protein derived from HopQ or YgjK protein, hence said HopQ or YgjK protein scaffolds containing the epitope specifically binding to the protein binding agents of the method. Said pair of protein binding agents specifically binding the scaffold protein epitopes present on a MegaBody as disclosed herein, or as disclosed in Steyaert et al. (WO2019/086548A1), or as may be described elsewhere has the further advantage that the method can be applied to capture or scavenge MegaBody-bound target proteins from complex mixtures, or may be applied as a generic selection tool, similar to other tagged target proteins.

Another embodiment relates to the method as described herein wherein the target protein comprises a tag or a heterologous tag or a label or detectable label. The term “detectable label” or “labelling”, or “tagging” refers to detectable labels or tags allowing the detection, visualization, and/or isolation, further purification and/or immobilization of the target protein of interest, or alternatively when present on the stripper, of the elution complex, or isolated or purified (poly-)peptides or complex described herein, and is meant to include any labels/tags known in the art for these purposes. In a further embodiment, protein binding agents specifically binding an epitope on a tag as present on a fusion protein comprising the target protein. Particularly preferred are fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase; and (other) enzymatic labels (e.g., peroxidase, alkaline phosphatase, beta-galactosidase, urease or glucose oxidase). Also included are affinity tags, such as chitin binding protein (CBP), maltose binding protein (MBP), glutathione-S-transferase (GST), poly(His) (e.g., 6× His or His6), Strep-Tag®, Strep-tag II® and Twin-Strep-Tag®; solubilization tags, such as thioredoxin (TRX), poly(NANP) and ubiquitin, or Small ubiquitin-like modifier (SUMO) or SMT3; chromatography tags, such as a FLAG-tag; epitope tags, such as V5-tag, myc-tag and HA-tag, or EPEA (CaptureSelect C-tag; U.S. Pat. No. 9,518,084B2), or furthermore intein-chitin binding domain (intein-CBD), Streptavidin/Biotin-based tags, His-Patch ThioFusion (thioredoxin based), or HaloTag, or even reporter tags, such as HRP, or Alkaline phosphatase. A number of non-limiting examples is provided for example in Kimple et al. (2015 Table 9.9.1). Also included are combinations of any of the foregoing labels or tags.

In further embodiment, the method as described herein may comprise a sample containing the target protein, to apply in step a), which is a ‘complex sample’ or complex mixture of proteins, and/or other components. Said sample may be an in vitro mixture of components, or a biological sample, wherein the term ‘biological sample’ refers to serum, urine, cells and tissues, among other body fluids. A ‘complex mixture’ may also be provided as a lysate or cellular extract, or any mixture of components containing the target protein, which may also be a recombinantly produced target protein that is optionally spiked into a complex mixture (as to capture certain conformations in different conditions or particular environments). ‘Complex samples’ may also be provided as whole proteomes or soluble proteomic solutions containing entire protein repertoires of an organism, cellular context or tissue, optionally corresponding to the proteome of a certain signaling cascade, a disease stage or condition and so forth.

In a specific embodiment, the ‘complex sample’ comprising the target protein as provided in step a) of the method has also been applied as an immunogen or antigen to immunize an animal for obtaining the ‘plurality of polypeptide binders’ used in step b) of the described method. In such a method, a display library comprising a plurality of polypeptide binders that is less- or partially- or unbiased is applied for the selection, rather than a biased library often only raised against a specific (single) antigen (see examples).

As mentioned above, the method as described herein may result in elution of the complex comprising not only the stripper and target protein bound to a selected polypeptide binder, but said complex may comprise further proteins bound to the target, especially when starting from a complex sample in step a), wherein the binding of the target protein to the surface via the first protein binding agent thus already resulted in binding of target+interactor to said surface. In such embodiments, the selection of protein binders upon adding the plurality of protein binders may thus result in identification of protein binders that bind directly or indirectly to the target protein. Direct binding means that there is a direct interaction and specificity for the target protein, whereas indirect binding means herein that said polypeptide binders binds to another component of the eluted protein complex, such as a protein (from the complex sample used in step a)) that in itself has been bound and is present in the complex binding the target protein. So in a specific embodiment, the method allows to not just identify polypeptide binders for the target protein, but also for its interactors co-immobilized in step a) of the method.

Finally, it should be clear to the skilled person how and when the method for selection of polypeptide binders is useful, as ample utilities can be identified, with as non-limiting examples the use for selecting specific binders from recombinant antibody libraries in drug discovery, as well as for epitope binning or identification of novel epitopes on a target, i.e. epitopes different from the known binders (such as the trapper and stripper). As envisaged herein, said selection method is suitable for medium- and even high-throughput purposes.

It is to be understood that although particular embodiments, specific configurations as well as materials and/or molecules, have been discussed herein for methods, samples and products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Introduction

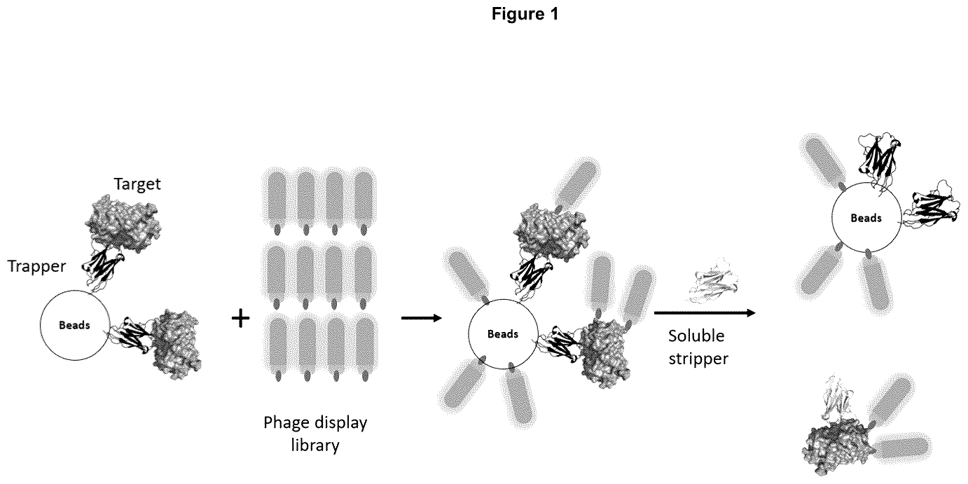

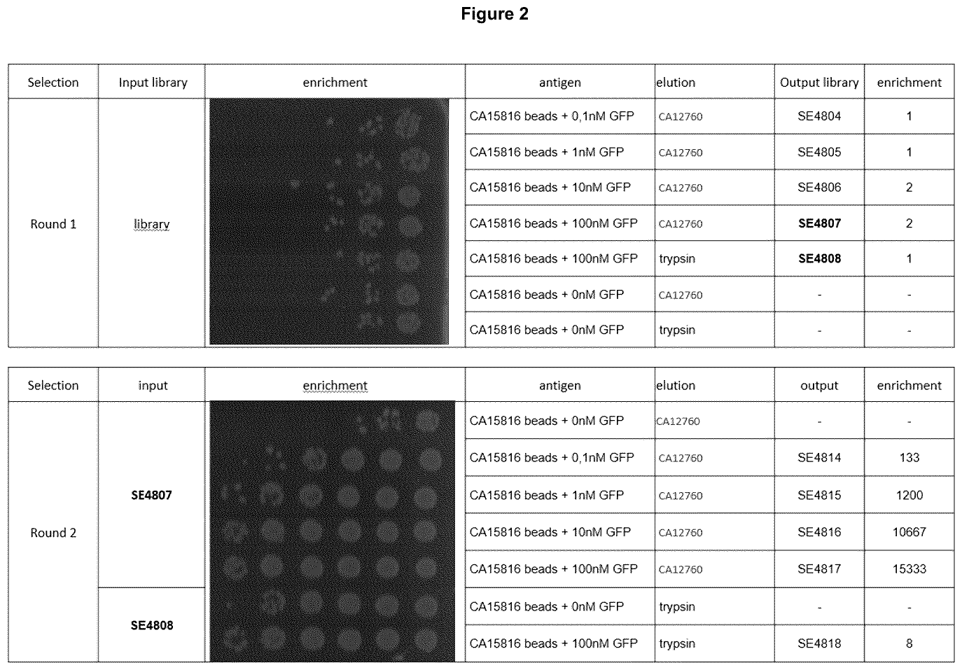

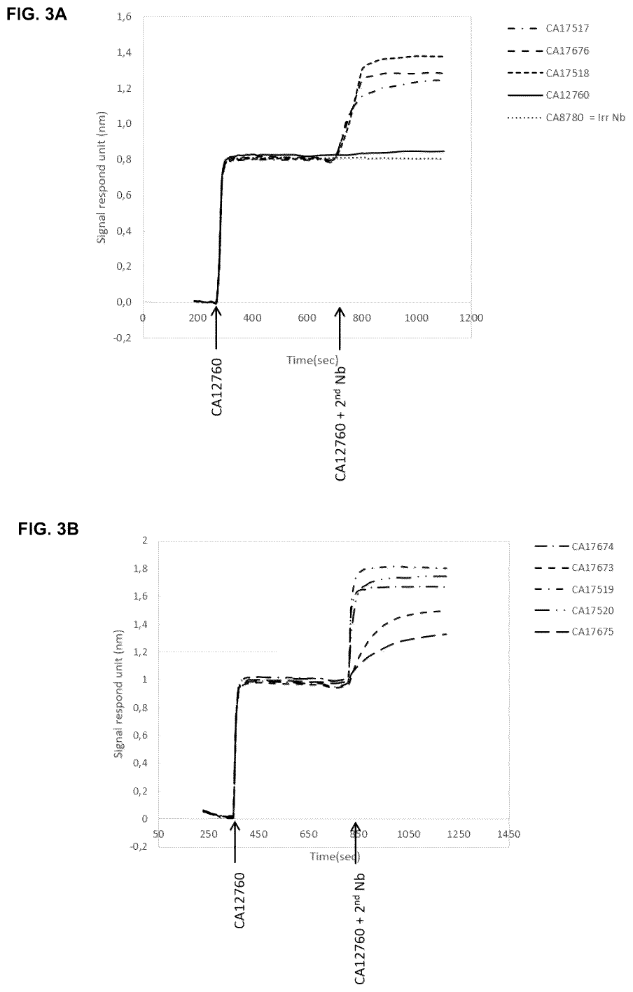

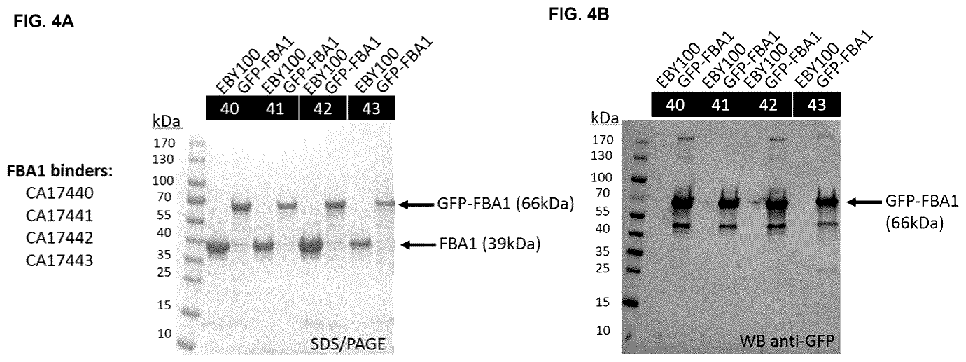

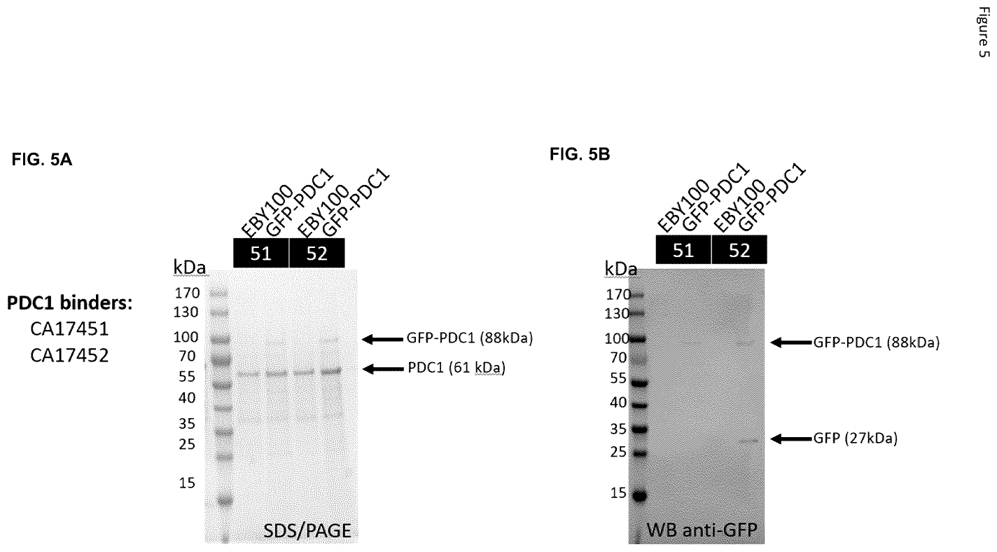

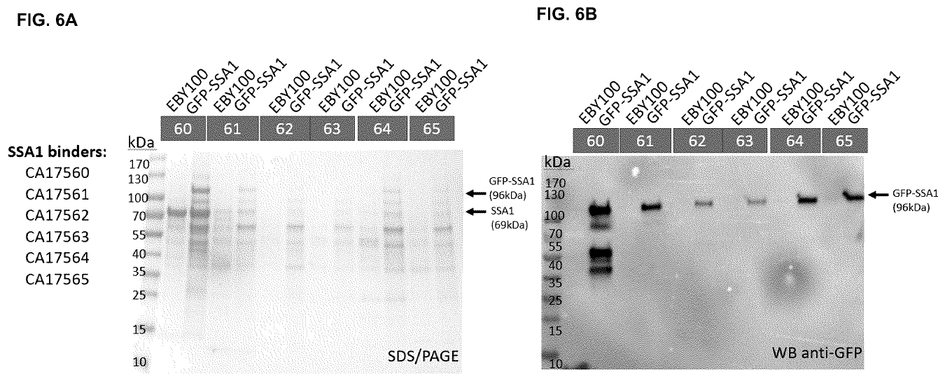

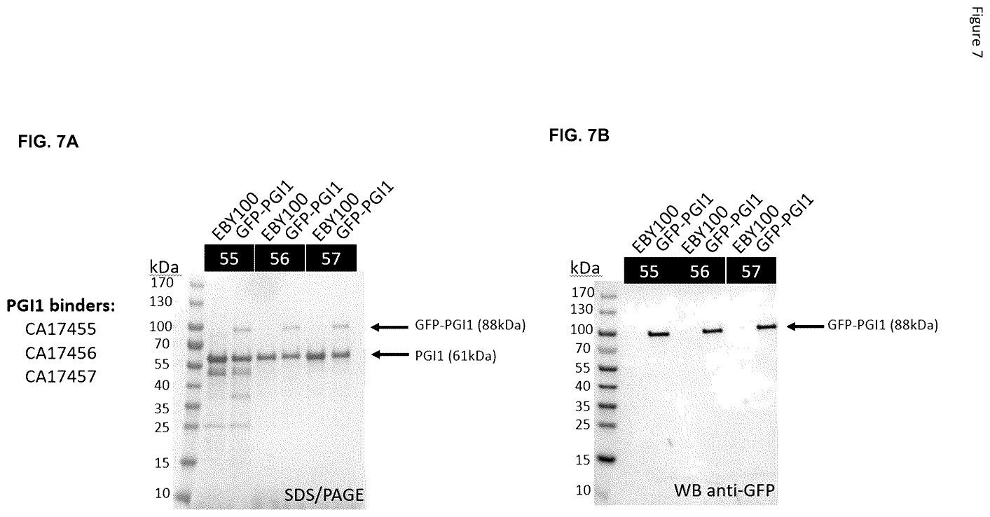

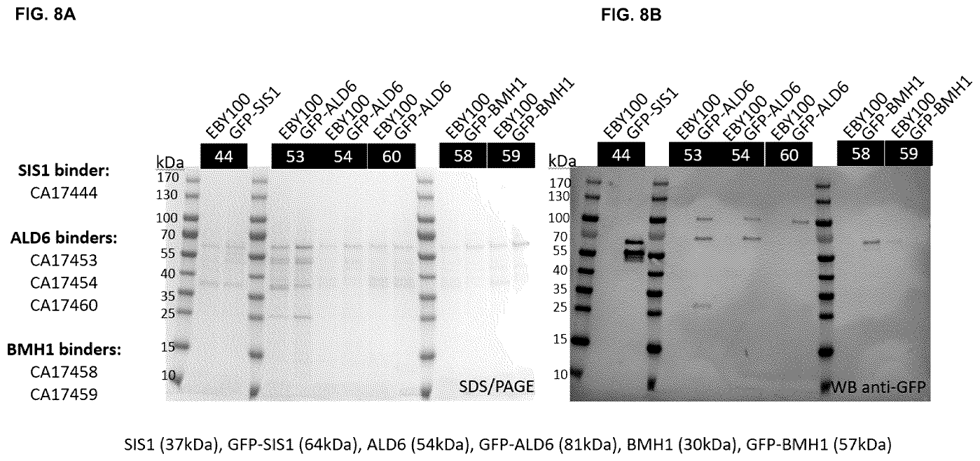

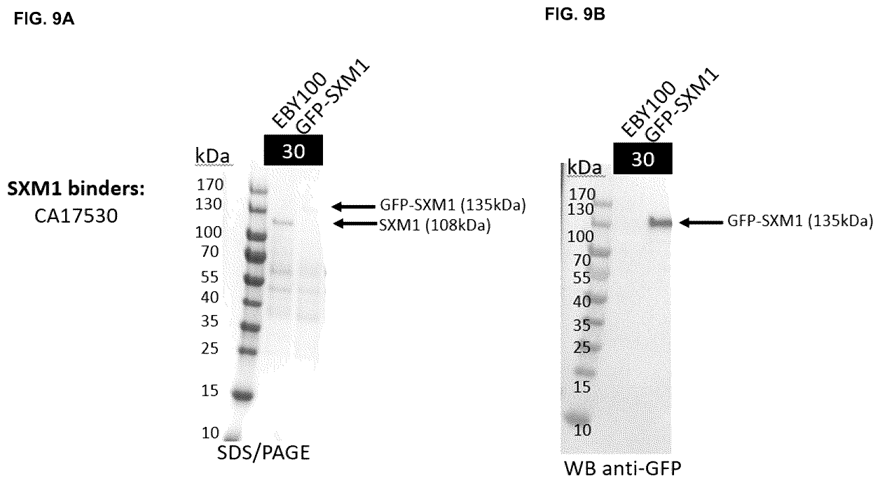

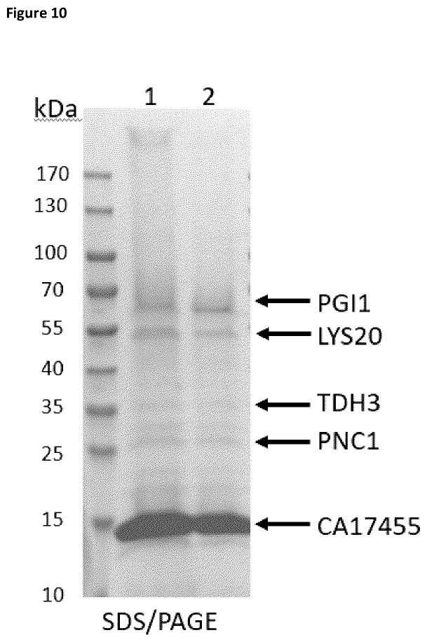

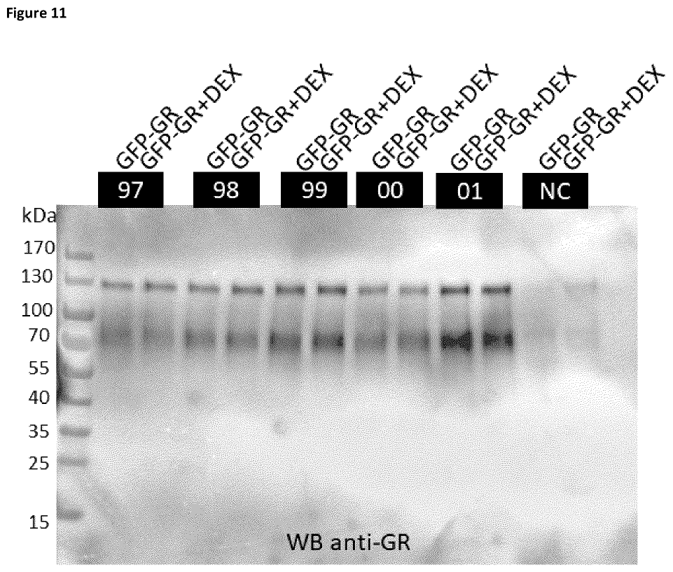

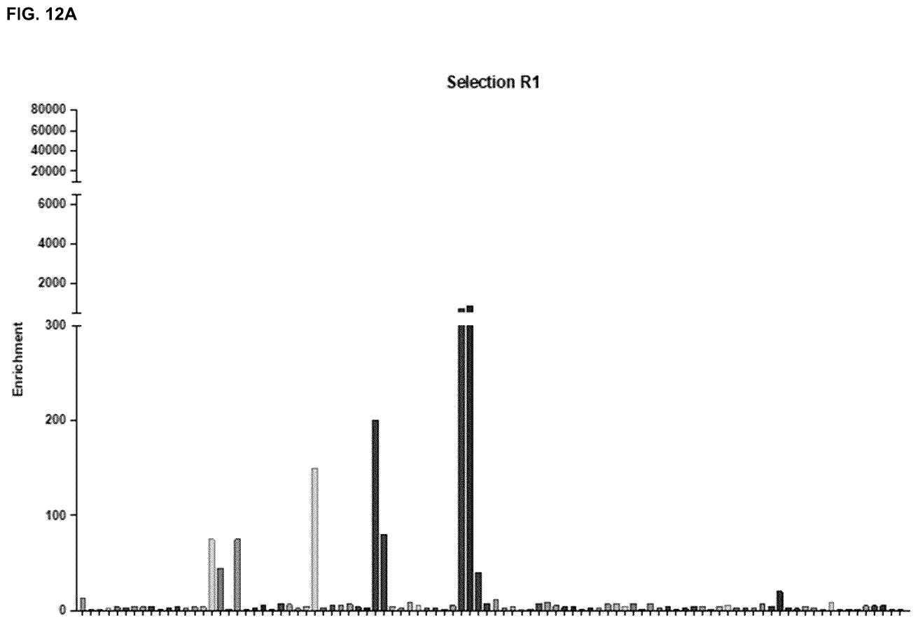

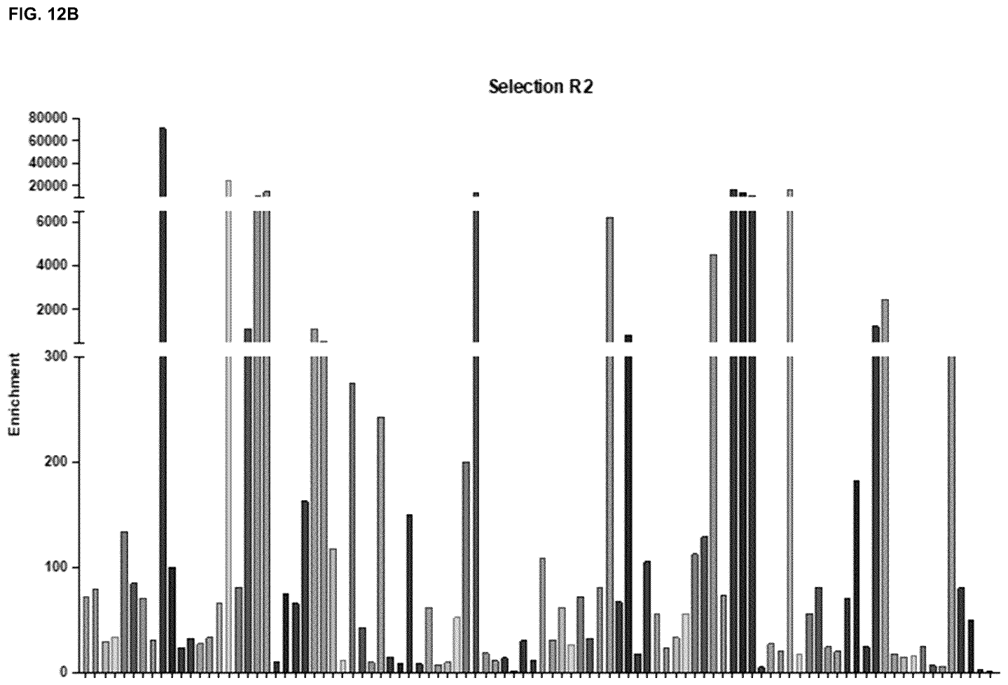

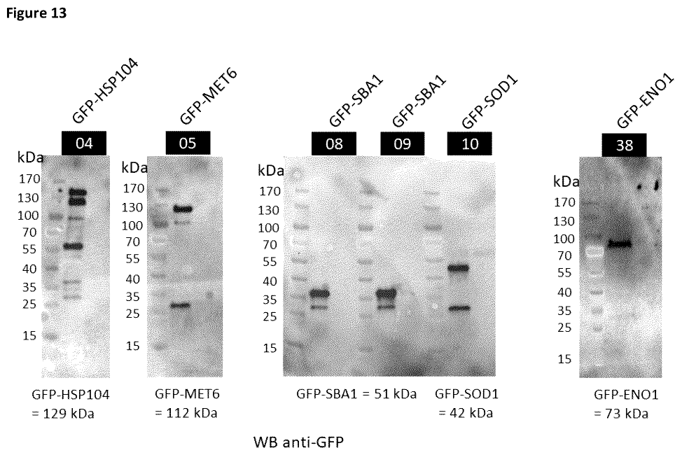

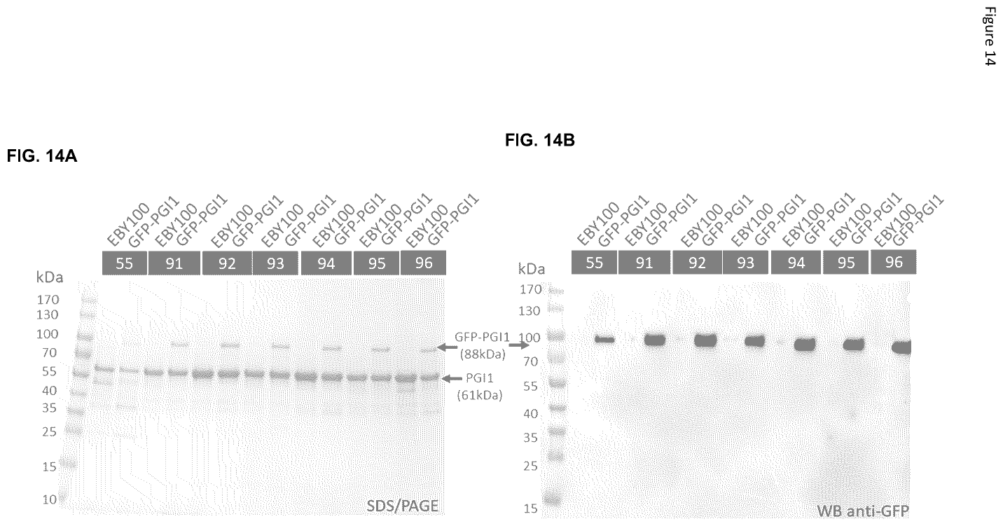

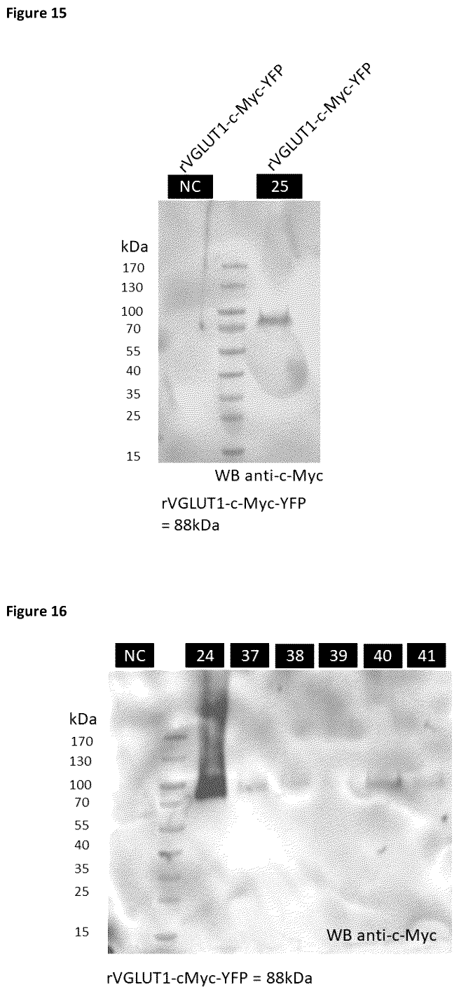

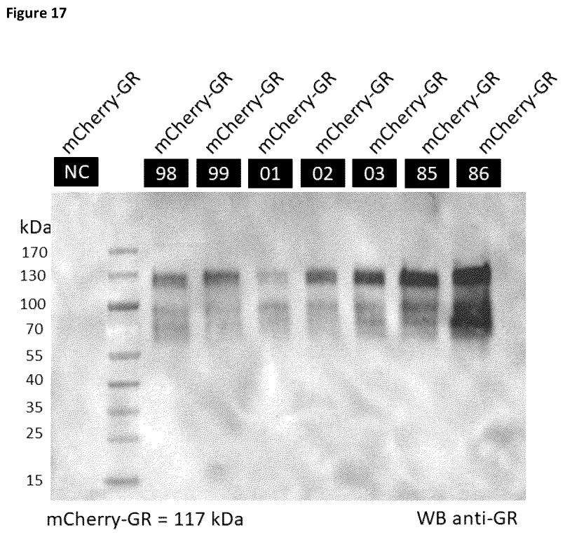

The present invention is based on the principle of the Nanobody exchange chromatography method (referred to herein as ‘NANEX’, based on the previously described method in PCT/EP2020/087291), which is applied herein for the selection of target specific binding agents like e.g. antibodies from display libraries. In particular, a first binding agent (referred to herein as the ‘trapper’), in particular a Nanobody, that is (preferably covalently) attached to a solid support is used to immobilize an antigen or target or protein of interest (as used interchangeably herein). The immobilized antigen is then incubated with a diverse repertoire of different binding agents (e.g. antibodies) that are expressed and displayed in a way to provide a physical link between each binding domain's phenotype (binding behavior) and the encoding genotype, for example by page display, yeast display, ribosome display, or any other method. Washing steps can optionally be used to remove irrelevant binding agents or antibodies. Similar to the NANEX purification method, and particular to this method, a second binding agent (referred to herein as the ‘stripper’ or ‘displacer’), in particular a Nanobody, that competes with the trapper for binding to the antigen is then used to selectively elute the immobilized antigen in association with the stripper and antigen-specific binding domain(s) or antibodies and their encoding genotype. Binders can then be amplified to generate a new repertoire enriched in binding agents or antibodies specific for the antigen, allowing to repeat this cycle until binders dominate the population and can be characterized as monoclonal binding domains or antibodies (FIG. 1). Thereby, a novel selection method for specific target protein binders is provided herein, which for the first time integrates the principle of NANEX-based immune displacement purification to increase efficiency and selectivity of the selection process.

### Example 1. NANEX to Select Novel GFP-Specific Nanobodies from an Antibody Library Using a GFP-Specific Trapper/Stripper Pair and GFP Protein as the Target

Generation of a GFP antibody library. A llama was immunized once a week for six times with in total 850 μg GFP (SEQ ID NO:25), and a Phage display library was generated as described (Materials and methods).

Discovery of novel GFP-specific Nanobodies. For the discovery of novel GFP-specific Nanobodies that bind non-overlapping epitopes, as compared to the epitopes of the trapper/stripper pair, we immobilized a GFP specific trapper with reference CA15816 (SEQ ID NO:2) that has an affinity for GFP of 2.7 nM on magnetic beads according to the manufacturer instructions. These trapper-coated beads were blocked and washed 5 times with PBS before use. Different concentrations ranging from 0.1 nM to 100 nM of GFP (SEQ ID NO:25) were mixed with these trapper-coated beads to trap GFP on the NANEX beads. Trapper-coated beads that were not incubated with GFP were used as a negative control. Upon incubation with GFP (the antigen), all magnetic beads were routinely washed 3 times and incubated in 96 well plates with the GFP antibody library displayed on phage. After an incubation period of 2 h at 4° C., beads were routinely washed 12 times with PBS-Tween. For the selection of novel GFP-specific antibodies that bind epitopes that do not overlap with those of the trapper/stripper pair, the non-covalent interaction between the trapper and GFP was selectively disrupted by adding a high-affinity stripper CA12760 (SEQ ID NO:1) that competitively binds to an epitope that overlaps with the trapper epitope. Therefore, the beads were incubated for 30 min with the high-affinity CA12760 (GFP-specific stripper) that binds the same epitope on GFP. GFP with bound phage was selectively eluted with this GFP-specific stripper. Phage were next amplified to generate a new repertoire enriched in antibodies. For comparison, we eluted bound phage with Trypsin from the trapper-coated beads prepared with 100 nM of GFP as described (Pardon et al., 2014).

For the 2^{nd} round of selection using NANEX, we followed the same strategy, using the output phage obtained from the trapper-coated beads prepared with 100 nM of GFP on all beads. The enrichment seen after 1 or 2 rounds of selection are shown in FIG. 2. A significantly higher background is observed after 2 rounds of selection if the elutions are performed by trypsin as compared to NANEX. Eight different GFP-specific Nanobody families were selected based on their CDR3 sequences (Nb clone CA17517 corresponding to SEQ ID NO:26, CA17518-SEQ ID NO:27, CA17519-SEQ ID NO:28, CA17520-SEQ ID NO:29, CA17673-SEQ ID NO:30, CA17674-SEQ ID NO:31, CA17675-SEQ ID NO:32, CA17676-SEQ ID NO:33).

Epitope analysis. To demonstrate that the newly selected Nanobody families bind a different epitope on the target as compared to the trapper/stripper epitope, we set out a Bio-layer interferometry (BLI) binding experiment. For this we loaded the Streptavidin biosensor with biotinylated GFP, next we allowed CA12760 (SEQ ID NO:1), the GFP-stripper with picomolar affinity, to bind to GFP. After washing, this biosensor loaded with the GFP-CA12760 complex was incubated with the newly discovered Nbs. In each case, we observed a further increase in mass, indicating that all these Nanobodies bind to another epitope and do not displace CA12760 (FIG. 3).

### Example 2. NANEX to Select Novel Target-Specific Nanobodies from an Antibody Library Using a GFP-Specific Trapper/Stripper Pair and a GFP-Tagged Target Protein

Trapper/stripper pairs as for example the GFP-specific pair that was introduced in Example 1 can also be used first to capture a GFP-tagged protein of interest from a complex mixture (for example a cell lysate) and to selectively elute the GFP-tagged protein of interest from a matrix (beads, plates, etc). Here, we phage displayed the Nanobody repertoire of an immune library raised against the Human Glucocorticoid receptor ligand binding domain (GR-LBD) by immunizing a llama. A GFP-specific trapper that was covalently attached to a solid support was used to immobilize a GFP-tagged version of GR. This immobilized antigen GFP-GR was then incubated with the Nanobody display library derived from the immunized animal using the KingFisher™ Flex Purification System (ThermoScientific). Similar to NANEX and particular to this invention, a GFP-specific stripper that competes with the trapper was then used to selectively elute the immobilized GFP-GR in association with LBD-specific Nanobodies displayed on phage. Phage that were selectively recovered with this GFP-specific stripper were next amplified to generate a new repertoire enriched in antibodies specific stripper were next amplified to generate a new repertoire enriched in antibodies (specifically Nbs herein) specific for the LBD, allowing to repeat this cycle until Nanobodies specific for the LBD dominated the population to be characterized as monoclonal LBD binders.

Generation of a GR-LBD antibody library. A construct encoding the ligand binding domain (LBD, 369-777) of the Human Glucocorticoid receptor (GR) was used to express recombinant LBD in the cytoplasm of E. coli in the presence of Dexamethasone (Dex). The GR-LBD domain was purified to homogeneity as a soluble protein by affinity purification (Ni-NTA) followed by dialysis in a buffer (20 mM NaH₂PO₄ PH 8.0, 150 mM NaCl, 10% glycerol, 1 mM DTT, and 10 μM Dex). A llama was immunized weekly over a period of 6 weeks with a total of 110 μg GR-LBD and a Phage display library was generated as described (Materials and methods).

Discovery of novel GR-LBD-specific Nanobodies. The full-length gene encoding the human Glucocorticoid receptor (NR3C1) was introduced in a pCDNA3 expression vector to generate a GFP-tagged fusion of the target. This vector was transfected in HEK293T cells using poly-ethyleneimine (PEI) as transfection agent. 48 h after transfection, 50 million cells were harvested, washed with ice cold PBS buffer and lysed using a Dounce homogenizer in 5 mL of lysis buffer (10 mM Hepes pH 7.4, 10 μM Dex, 10% glycerol, 10 μM ZnCl₂, 2.5 mM MgCl₂, 2.5 mM DTT, 0.5% NP40 substitute) supplemented with 50 μg/ml DNAse I and protease inhibitors. The lysate was clarified by centrifugation at 20,000 g. The supernatant containing the GFP-tagged GR (the target) was collected and incubated for 1 h at 4° C. in a 96 well deepwell block with 20 μL of magnetic beads that were covalently functionalized with the GFP-specific trapper to immobilize the GFP-tagged GR on these beads. Using the KingFisher flex device, these beads were collected and washed with 0.5 ml of washing buffer (10 mM Hepes pH 7.4, 10 μM Dex, 10% glycerol, 10 UM ZnCl₂, 2.5 mM MgCl₂, 2.5 mM DTT) before they were incubated for 1 h at 4° C. with the phage displaying the GR-LBD Nanobody library (1,4·10¹⁴ phages). Next, these beads were washed 9 times with 0.5 mL of washing buffer. Next, the GFP-specific stripper Nanobody was then used to selectively elute the immobilized GR in association with GR-specific Nanobodies displayed on phage. Eluted phage were used to infect exponentially grown E. coli TG1 cells and incubated for 30 min at 37° C. without shaking. Next, LB medium (supplement with 100 μg/mL ampicillin and 2% wt/vol glucose) was added and the culture grown overnight at 37° C. The following day the culture was centrifuged at 3000 g, the cell pellet was resuspended in LB (supplemented with 100 μg/mL ampicillin and 20% glycerol (vol/vol)) and stored at −80° C. as a glycerol stock for later use. An un-transfected HEK293T lysate (which does not contain any GFP-tagged protein) was spiked with 20 μg of wild type GFP to be used as the (negative) control.

After 3 rounds of selection by panning using NANEX technology, individual clones were isolated from the enriched sub-library by plating tenfold serial dilutions of the overnight-grown phage-infected TG1 cells in LB. 96 individual clones were picked and grown in a 96-well plate containing LB supplemented with 100 μg/mL ampicillin. Plasmids containing the Nanobody encoding genes were purified, sequenced and grouped into sequence families (Material and methods). A representative member of each sequence family was selected and the encoding plasmid was transformed into the E. coli WK6 expression strain to express and purify each Nanobody of interest (Nb clone CA17797 corresponding to SEQ ID NO:35, CA17798-SEQ ID NO:36, CA17799-SEQ ID NO:37, CA17800-SEQ ID NO:38, CA17801-SEQ ID NO:39). The specificity of the GR-specific Nanobodies was verified by linking separate Nanobodies on NHS-agarose beads for co-immunoprecipitation assays. The beads that were functionalized with these GR-specific Nanobodies were incubated for an hour at 4° C. on a rotating device with a lysate of HEK293T cells transfected with the GFP-GR vector. After washing, these beads were resuspended in SDS-PAGE loading dye and analyzed on SDS-PAGE. Separated proteins were transferred to a PVDF membrane and the Western blot was developed with a GR-specific antibody to confirm the presence of GFP-tagged GR (FIG. 11).

From this example we conclude that the NANEX-based selection method allows to easily select target-specific antibodies from an antibody library using a GFP-specific trapper/stripper pair and a GFP-tagged target protein.

### Example 3. NANEX to Select Novel Target-Specific Binders from a Proteome-Wide Antibody Library Using a GFP-Specific Trapper/Stripper Pair and a GFP-Tagged Target

Trapper/stripper pairs as for example the GFP-specific pair that was introduced in Example 1 can also be used, first to capture a GFP-tagged protein of interest from a complex mixture (for example a cell lysate) on a matrix (beads, plates, etc.). Similar to NANEX and particular to this method, a second Nanobody (the stripper) that competes with the trapper is then used to selectively elute the immobilized antigen in association with antigen-specific binding domains or antibodies and their encoding genotype.

For Example 3, we immunized a llama with all the soluble yeast proteins (soluble yeast proteome) to raise an immune response against all soluble yeast proteins. The Nanobody repertoire of this immunized animal was displayed on phage to generate a proteome-wide antibody library. In parallel, a GFP-specific trapper that was covalently attached to a solid support was used to immobilize the GFP-tagged version of FBA1 (Huh et al., 2003). This immobilized antigen (GFP-FBA1) was then incubated with the Nanobody display library derived from the immunized animal using a KingFisher Flex device.

Similar to NANEX and particular to this invention, a GFP-specific stripper that competes with the trapper was then used to selectively elute the immobilized GFP-FBA1 in association with FBA1-specific Nanobodies displayed on phage. Phage that were selectively recovered with this GFP-specific stripper were next amplified to generate a new repertoire enriched in antibodies specific for FBA1, allowing to repeat this cycle until Nanobodies specific for FBA1 dominated the population to be characterized as monoclonal FBA1 binders.

Generation of a proteome-wide antibody library. A llama was immunized weekly over a period of 6 weeks with a total of 3 mg of all the soluble yeast proteins (soluble proteome) of Saccharomyces cerevisiae strain EBY100 (ATCC® MYA-4941™) to generate a proteome-wide antibody library. To prepare this proteome-wide mixture of soluble antigens, a 1-liter culture of EBY100 was grown in YPD media and harvested at mid-log phase (OD600=0.6). Cells were collected by centrifugation and resuspended in PBS supplemented with 50 μg/ml DNAse I and EDTA-free protease inhibitors (Complete™ Roche). Cells were then lysed using a French press and centrifuged for 30 min at 20,000 g. The supernatant containing all soluble proteins was collected, syringe filtered using a 0.22 μm filter, aliquoted and stored at −80° C. The total protein concentration in the lysate was 1.5 mg/ml as determined by BCA protein quantification (Pierce™ BCA Protein Assay Kit, 23225, ThermoFisher). After immunization, a Phage display library was generated as described (Materials and methods).

Discovery of novel FBA1-specific Nanobodies. We used NANEX to selectively capture GFP-FBA1 from a lysate of an engineered yeast strain (Yeast GFP fusion collection reference: GFP(+)22, G1) expressing FBA1 (Systematic Name YKL060C) as a GFP-tagged protein (Huh et al., 2003) on trapper-coated magnetic beads (see Example 1). These magnetic beads were washed, incubated with the phage displaying the proteome-wide Nanobody library (1,4·10¹⁴ phages) and washed again. Next, the GFP-specific stripper Nanobody was then used to selectively elute the immobilized GFP-FBA1 in association with FBA1-specific Nanobodies displayed on phage using the KingFisher Flex device. Eluted phage were used to infect exponentially grown E. coli TG1 cells and incubated for 30 min at 37° C. without shaking. Next, LB medium (supplement with 100 μg/mL ampicillin and 2% wt/vol glucose) was added and the culture grown overnight at 37° C. The following day the culture was centrifuged at 3000 g, the cell pellet was resuspended in LB (supplement with 100 μg/mL ampicillin and 20% glycerol (vol/vol)) and stored at −80° C. as a glycerol stock for later use. Yeast lysate from the reference strain EBY100 (which does not contain any GFP-tagged protein) was spiked with 20 μg of wild type GFP to be used as the (negative) control.

Characterization of FBA1-specific Nanobodies. After 3 rounds of selection by panning using NANEX technology, individual clones were isolated from the enriched sub-library by plating tenfold serial dilutions of the overnight-grown phage-infected E. coli TG1 cells in LB. 96 individual clones were picked and grown in a 96-well plate containing LB supplemented with 100 μg/mL ampicillin. Plasmids containing the Nanobody encoding genes were purified, sequenced and grouped into sequence families (Material and methods). A representative member of each sequence family was selected. Four different FBA1-specific Nanobodies, derived from four different sequence families were selected based on their CDR3 sequences (Nb clone CA17440 corresponding to SEQ ID NO:3, CA17441-SEQ ID NO:4, CA17442-SEQ ID NO:5, and CA17443-SEQ ID NO:6). Their encoding plasmid was transformed into the E. coli WK6 expression strain to express and purify each Nanobody of interest. The specificity of the FBA1-specific Nanobodies was confirmed by linking separate Nanobodies on NHS-agarose beads for co-immunoprecipitation assays. The beads that were functionalized with these FBA1-specific Nanobodies and incubated for an hour at 4° C. on a rotating device with either an EBY100 lysate expressing native FBA1 or the lysate of the engineered yeast strain (GFP(+)22, G1) expressing FBA1 as a GFP-tagged protein. After washing, these beads were resuspended in SDS-PAGE loading dye and analyzed on SDS-PAGE (FIG. 4). Separated proteins were also transferred to a PVDF-membrane and the Western blot was developed with a GFP-specific antibody to confirm the presence of the GFP-tagged FBA1 (FIG. 4). For each FBA1-specific Nanobody analyzed in this way, co-immunoprecipitation with the EBY100 lysate gives a predominant band observed at 39 kDa, corresponding to the expected molecular weight of FBA1. Moreover, when incubated with the lysate of the Yeast strain expressing FBA1 as a GFP-tagged protein the predominant band is observed at 66 kDa, consistent with the molecular weight of GFP-FBA1 (27 kDa+39 kDa=66 kDa). Western blot analysis with anti-GFP antibodies confirmed that the 66 kDa band contains the GFP-tag. The identity of FBA1 was further confirmed by analyzing the relevant bands (39 kDa and 66 kDa) by mass spectrometry.

From this example we conclude that the NANEX-based selection method allows to easily select target-specific antibodies from proteome-wide antibody libraries.

### Example 4. NANEX for the Parallel Selection of Target-Specific Binders from a Proteome-Wide Antibody Library Using a GFP-Specific Trapper/Stripper Pair and Separate GFP-Tagged Targets

To proof that the NANEX-based selection method as described herein is broadly applicable for the discovery of antibodies against diverse targets, we selected specific binders for 12 different soluble Yeast proteins (Table 1) from a proteome-wide antibody library following a parallel approach. Therefore, 12 engineered yeast strains, each expressing another GFP-tagged protein of interest (Yeast GFP Fusion Collection, ThermoFisher) were selected. These proteins were chosen according to their abundance in S. cerevisiae, spanning from one of the most abundant proteins in Yeast, FBA1 (YKL060C), to RIF2 (YLR453C), a protein that controls telomere length and that has less than a thousand molecules per cell (SGD Project. http://www.yeastgenome.org).

TABLE 1
Properties of 12 GFP-tagged proteins that were used as
representative targets of the soluble yeast proteome.

| Standard | Systematic | Median Abundance | Yeast GFP Fusion |
|---|---|---|---|
| Name | Name | (molecules/cell) | Collection reference |
| FBA1 | YKL060C | 737009 | GFP(+)22, G1 |
| PDC1 | YLR044C | 581219 | GFP(+)12, F8 |
| SSA1 | YAL005C | 255901 | GFP(+)10, E4 |
| PGI1 | YBR196C | 158738 | GFP(+)12, H11 |
| ALD6 | YPL061W | 135000 | GFP(+)15, F1 |
| BMH1 | YER177W | 65471 | GFP(+)27, D5 |
| ACC1 | YNR016C | 29049 | GFP(+)01, B2 |
| SIS1 | YNL007C | 28050 | GFP(+)22, E5 |
| SXM1 | YDR395W | 7202 | GFP(+)04, H9 |
| TOP2 | YNL088W | 3960 | GFP(+)01, H11 |
| TAM41 | YGR046W | 1666 | GFP(+)20, A8 |
| RIF2 | YLR453C | 976 | GFP(+)20, E1 |

The GFP-specific trapper that was introduced in Example 1 was used to separately capture each GFP-tagged target from a cell lysate of the corresponding engineered yeast strain on beads. These different beads were next incubated separately with the proteome-wide antibody library and the GFP-specific stripper was used to selectively elute the different GFP-tagged proteins of interest in association with antigen-specific binding domains or antibodies and their encoding genotype. Phage that were recovered with this GFP-specific stripper were next amplified to generate a new repertoire enriched in antibodies specific for each target protein or protein of interest (POI), allowing to repeat this cycle until Nanobodies specific for the POI dominated the population to be characterized as monoclonal binders.

Generation of a proteome-wide antibody library. The same library that was described under Example 3 was also used to perform the experiments described in Example 4.

Discovery of novel POIs-specific Nanobodies. Similar to Examples 1, 2 and 3 we used NANEX to selectively capture 12 different GFP-POIs from lysates of engineered Yeast strains expressing the different POIs (Table 1) as GFP-tagged proteins (Huh et al., 2003) on 12 separate trapper-coated magnetic beads. After washing, these separate magnetic beads were incubated with the phage displaying the proteome-wide Nanobody library (1,4·10¹⁴ phages) and washed again. Particular for this example, the GFP-specific stripper Nanobody was then used to selectively elute the immobilized GFP-POIs in association with POIs-specific Nanobodies displayed on phage. Selectively recovered phage were used to infect exponentially grown E. coli TG1 cells and incubated for 30 min at 37° C. without shaking. Next, LB medium (supplemented with 100 μg/mL ampicillin and 2% wt/vol glucose) was added and the culture grown overnight at 37° C. The following day the culture was centrifuged at 3000 g, the cell pellet was resuspended in LB (supplement with 100 μg/mL ampicillin and 20% glycerol (vol/vol)) and stored at −80° C. as a glycerol stock for later use. Yeast lysate from the reference strain EBY100 (which does not contain any GFP-tagged protein) was spiked with 20 μg of wild type GFP to be used as the (negative) control.

For each target selection of binders in this example, as detailed in Table 2, after 3 rounds of selection by panning using NANEX technology, individual clones were isolated from the enriched sub-library by plating tenfold serial dilutions of the overnight-grown phage-infected E. coli TG1 cells in LB. 96 individual clones were picked and grown in a 96-well plate containing LB supplemented with 100 μg/ml ampicillin. Plasmids containing the Nanobody encoding genes were purified, sequenced and grouped into sequence families (Material and methods). A representative member of each sequence family was selected. Their encoding plasmid was transformed into the E. coli WK6 expression strain to express and purify each Nanobody of interest. The specificity of the target-specific Nanobodies was confirmed by linking separate Nanobodies on NHS-agarose beads for co-immunoprecipitation assays. The beads that were functionalized with these target-specific Nanobodies were incubated for one hour at 4° C. on a rotating device with either an EBY100 lysate expressing native target protein or the lysate of the engineered Yeast strain (Yeast GFP fusion collection reference, as specified in Table 2) expressing the target protein as a GFP-tagged protein. After washing, these beads were resuspended in SDS-PAGE loading dye and analyzed on SDS-PAGE (as in the Figure specified in Table 2 for each target). Separated proteins were transferred to a PVDF membrane and the Western blot was developed with GFP-specific antibody to confirm the presence of GFP-tagged target. For each target-specific Nanobody analysed in this way, the co-immunoprecipitation with the EBY100 lysate gives a predominant band corresponding to the expected molecular weight of the target protein. Moreover, when incubated with the lysate of the Yeast strain expressing target as a GFP-tagged protein the predominant band is observed at a MW consistent with the molecular weight of GFP-target. Western blot anti-GFP confirmed that the latter band contains a GFP tag.

TABLE 2
Yeast protein targets used for selection of Nb binders (as described in Example 4).

| | | | | SDS- | MW | MW GFP- |
|---|---|---|---|---|---|---|
| Target | Yeast | # Nb | Selected Nb | PAGE/ | target | target |
| protein | collection ref. | families | sequences | WB FIG. | (kDa) | (kDa) |
| PDC1 | GFP(+)12, F8 | 2 | CA17451 | FIG. 5 | 61 | 88 |
| | | | SEQ ID NO: 7 | | | |
| | | | CA17452 | | | |
| | | | SEQ ID NO: 8 | | | |
| SSA1 | GFP(+)10, E4 | 6 | CA17560 | FIG. 6 | 69 | 96 |
| | | | SEQ ID NO: 19 | | | |
| | | | CA17561 | | | |
| | | | SEQ ID NO: 20 | | | |
| | | | CA17562 | | | |
| | | | SEQ ID NO: 21 | | | |
| | | | CA17563 | | | |
| | | | SEQ ID NO: 22 | | | |
| | | | CA17564 | | | |
| | | | SEQ ID NO: 23 | | | |
| | | | CA17565 | | | |
| | | | SEQ ID NO: 24 | | | |
| PGI1 | GFP(+)12, H11 | 3 | CA17455 | FIG. 7 | 61 | 88 |
| | | | SEQ ID NO: 15 | | | |
| | | | CA17456 | | | |
| | | | SEQ ID NO: 16 | | | |
| | | | CA17457 | | | |
| | | | SEQ ID NO: 17 | | | |
| SIS1 | GFP(+)22, E5 | 1 | CA17444 | FIG. 8 | 37 | 64 |
| | | | SEQ ID NO: 9 | | | |
| ALD6 | GFP(+)15, F1 | 3 | CA17453 | FIG. 8 | 54 | 81 |
| | | | SEQ ID NO: 10 | | | |
| | | | CA17454 | | | |
| | | | SEQ ID NO: 11 | | | |
| | | | CA17460 | | | |
| | | | SEQ ID NO: 12 | | | |
| BMH1 | GFP(+)27, D5 | 2 | CA17458 | FIG. 8 | 30 | 57 |
| | | | SEQ ID NO: 13 | | | |
| | | | CA17459 | | | |
| | | | SEQ ID NO: 14 | | | |
| SXM1 | GFP(+)04, H9 | 1 | CA17530 | FIG. 9 | 108 | 135 |
| | | | SEQ ID NO: 18 | | | |

From Example 4, we conclude that NANEX allows to easily select target-specific antibodies against several different targets from proteome-wide antibody libraries.

### Example 5. High-Throughput NANEX for the Parallel Selection of Target-Specific Binders from a Proteome-Wide Antibody Library Using a GFP-Specific Trapper/Stripper Pair and Separate GFP-Tagged Targets

Example 4 describes the parallel selection of antigen-specific antibodies for different targets from a proteome-wide antibody library using NANEX with a GFP-specific trapper/stripper pair. In Example 5 we have scaled up this process for the discovery of antigen-specific binders for 94 different GFP-tagged antigens from a proteome-wide antibody library following a parallel (high-throughput) approach. 96 different selections were performed in parallel using a KingFisher instrument (ThermoFisher Scientific), allowing most of the steps to be automated.

Therefore, 94 representative yeast strains, each expressing another GFP-tagged protein of interest (Yeast GFP Fusion Collection, ThermoFisher) were selected according to the MW and the abundance of the protein (Table 3). 2 negative controls (yeast without a GFP fusion protein and lysis buffer without yeast lysate) were added to the 96 well plate.

As for Example 3 and 4, we immunized a Llama with all the soluble yeast proteins (soluble yeast proteome) to raise an immune response against all soluble yeast proteins. The Nanobody repertoire of this immunized animal was displayed on phage to generate a proteome-wide antibody library.

The GFP-specific trapper CA15816 (SEQ ID NO:2) that was introduced in Example 1 was covalently attached to a solid support (magnetic beads) and dispensed in 96 different wells. Each condition was used to separately capture each GFP-tagged target from a cell lysate of the corresponding engineered yeast strain on beads. Captured GFP-tagged targets were incubated separately with the proteome-wide antibody library from Example 3 and 4 and the GFP-specific stripper CA12760 (SEQ ID NO:1) was used to selectively elute the different GFP-tagged proteins of interest in association with antigen-specific binding domains or antibodies and their encoding genotype. Phage that were selectively recovered with this GFP-specific stripper were next amplified to generate a new repertoire enriched in antibodies specific for each POI, allowing to repeat this cycle until Nanobodies specific for the POI dominated the population to be characterized as monoclonal binders.

TABLE 3
94 representative yeast strains, each expressing a GFP-tagged protein of interest.

| | | | | MW | MW GFP- | |
|---|---|---|---|---|---|---|
| | | Target | Yeast | target | target | |
| Well | ORF name | protein | collection ref. | (kDa) | (kDa) | Molecules/cell |
| A1 | YLL026W | HSP104 | GFP(+)05, A2 | 102 | 129 | 35086 +/− 12172 |
| B1 | YPR160W | GPH1 | GFP(+)05, G2 | 103 | 130 | 8611 +/− 2558 |
| C1 | YDR074W | TPS2 | GFP(+)05, C4 | 103 | 130 | 16558 +/− 6235 |
| D1 | YER091C | MET6 | GFP(+)07, D4 | 86 | 113 | 111404 +/− 75050 |
| E1 | YOR361C | PRT1 | GFP(+)07, A6 | 88 | 115 | 26937 +/− 9088 |
| F1 | YGR264C | MES1 | GFP(+)07, A9 | 86 | 113 | 29753 +/− 10302 |
| G1 | YAL043C | PTA1 | GFP(+)07, C1 | 88 | 115 | 2822 +/− 637 |
| H1 | YGL009C | LEU1 | GFP(+)07, C2 | 86 | 113 | 56013 +/− 24899 |
| A2 | YFR009W | GCN20 | GFP(+)07, F8 | 85 | 112 | 13923 +/− 2477 |
| B2 | YKL054C | DEF1 | GFP(+)07, F11 | 84 | 111 | 20936 +/− 16375 |
| C2 | YIL078W | THS1 | GFP(+)07, F12 | 85 | 112 | 29362 +/− 10889 |
| D2 | YLR153C | ACS2 | GFP(+)09, B4 | 75 | 102 | 42561 +/− 24760 |
| E2 | YPR074C | TKL1 | GFP(+)09, G4 | 74 | 101 | 73106 +/− 45786 |
| F2 | YDR129C | SAC6 | GFP(+)09, H4 | 72 | 99 | 25994 +/− 5216 |
| G2 | YBR121C | GRS1 | GFP(+)09, H8 | 75 | 102 | 42610 +/− 6183 |
| H2 | YLR044C | PDC1 | GFP(+)12, F8 | 61 | 88 | 581219 +/− 457355 |
| A3 | YER165W | PAB1 | GFP(+)12, B2 | 64 | 91 | 62422 +/− 31155 |
| B3 | YHR064C | SSZ1 | GFP(+)12, G4 | 58 | 85 | 41718 +/− 10307 |
| C3 | YPR145W | ASN1 | GFP(+)12, A5 | 64 | 91 | 48488 +/− 20357 |
| D3 | YMR105C | PGM2 | GFP(+)12, B6 | 63 | 90 | 21225 +/− 17383 |
| E3 | YGL076C | RPL7A | GFP(+)12, D12 | 28 | 55 | 78377 +/− 26064 |
| F3 | YCR053W | THR4 | GFP(+)14, C4 | 57 | 84 | 48158 +/− 10714 |
| G3 | YGR155W | CYS4 | GFP(+)14, E8 | 56 | 83 | 45620 +/− 13902 |
| H3 | YAL038W | CDC19 | GFP(+)14, G12 | 55 | 82 | 144590 +/− 77748 |
| A4 | YHR183W | GND1 | GFP(+)15, A7 | 54 | 81 | 107317 +/− 65557 |
| B4 | YER133W | GLC7 | GFP(+)15, A8 | 36 | 63 | 14600 +/− 7042 |
| C4 | YDR381W | YRA1 | GFP(+)15, D12 | 25 | 52 | 26822 +/− 10902 |
| D4 | YBR196C | PGI1 | GFP(+)12, H11 | 61 | 88 | 158738 +/− 89784 |
| E4 | YBR126C | TPS1 | GFP(+)15, E3 | 56 | 83 | 20541 +/− 7422 |
| F4 | YFR053C | HXK1 | GFP(+)15, E9 | 54 | 81 | 40800 +/− 31783 |
| G4 | YGL253W | HXK2 | GFP(+)15, F8 | 54 | 81 | 136905 +/− 64001 |
| H4 | YNR001C | CIT1 | GFP(+)16, C1 | 53 | 80 | 11990 +/− 4866 |
| A5 | YDL103C | QRI1 | GFP(+)16, G1 | 53 | 80 | 6707 +/− 3561 |
| B5 | YOR198C | BFR1 | GFP(+)16, F4 | 55 | 82 | 33400 +/− 10203 |
| C5 | YBR118W | TEF2 | GFP(+)16, B12 | 50 | 77 | 532551 +/− 389013 |
| D5 | YPR080W | TEF1 | GFP(+)16, F12 | 50 | 77 | 533920 +/− 332537 |
| E5 | YER043C | SAH1 | GFP(+)17, C6 | 49 | 76 | 78556 +/− 27388 |
| F5 | YBR143C | SUP45 | GFP(+)17, C12 | 49 | 76 | 36685 +/− 5922 |
| G5 | YGR254W | ENO1 | GFP(+)17, D12 | 47 | 74 | 99031 +/− 64617 |
| H5 | YDL091C | UBX3 | GFP(+)17, F1 | 53 | 80 | 1356 +/− 829 |
| A6 | YOR375C | GDH1 | GFP(+)17, G2 | 50 | 77 | 55615 +/− 32892 |
| B6 | YPR163C | TIF3 | GFP(+)18, G1 | 49 | 76 | 24565 +/− 6617 |
| C6 | YGR285C | ZUO1 | GFP(+)18, C3 | 49 | 76 | 46597 +/− 15022 |
| D6 | YLR438W | CAR2 | GFP(+)18, C9 | 46 | 73 | 21955 +/− 10239 |
| E6 | YCR012W | PGK1 | GFP(+)19, F1 | 45 | 72 | 535389 +/− 420545 |
| F6 | YIL033C | BCY1 | GFP(+)19, H1 | 47 | 74 | 10285 +/− 2495 |
| G6 | YKR059W | TIF1 | GFP(+)20, C1 | 45 | 72 | 52628 +/− 8788 |
| H6 | YGL105W | ARC1 | GFP(+)21, A2 | 42 | 69 | 38196 +/− 5218 |
| A7 | YPL061W | ALD6 | GFP(+)15, F1 | 54 | 81 | 135000 +/− 101795 |
| B7 | YLR048W | RPS0B | GFP(+)21, H4 | 28 | 55 | 88691 +/− 70495 |
| C7 | YPR035W | GLN1 | GFP(+)21, A6 | 42 | 69 | 38231 +/− 37111 |
| D7 | YPL081W | RPS9A | GFP(+)21, D10 | 22 | 49 | 66820 +/− 32245 |
| E7 | YDR012W | RPL4B | GFP(+)21, G11 | 39 | 66 | 36864 +/− 13336 |
| F7 | YDL055C | PSA1 | GFP(+)21, A12 | 40 | 67 | 104294 +/− 58655 |
| G7 | YJR105W | ADO1 | GFP(+)23, A3 | 36 | 63 | 41323 +/− 9660 |
| H7 | YMR142C | RPL13B | GFP(+)23, A8 | 23 | 50 | 47410 +/− 8017 |
| A8 | YJR009C | TDH2 | GFP(+)23, A9 | 36 | 63 | 171296 +/− 120482 |
| B8 | YKL085W | MDH1 | GFP(+)23, D7 | 36 | 63 | 20985 +/− 13089 |
| C8 | YBR189W | RPS9B | GFP(+)23, D8 | 22 | 49 | 70257 +/− 56063 |
| D8 | YCR036W | RBK1 | GFP(+)23, E7 | 37 | 64 | 3462 +/− 978 |
| E8 | YGR086C | PIL1 | GFP(+)23, F3 | 38 | 65 | 29832 +/− 9050 |
| F8 | YLR354C | TAL1 | GFP(+)23, F6 | 37 | 64 | 44083 +/− 13889 |
| G8 | YGR192C | TDH3 | GFP(+)23, F8 | 36 | 63 | 746358 +/− 666753 |
| H8 | YNL097C | PHO23 | GFP(+)23, F9 | 37 | 64 | 1688 +/− 627 |
| A9 | YDR044W | HEM13 | GFP(+)23, F10 | 38 | 65 | 5996 +/− 1996 |
| B9 | YJL052W | TDH1 | GFP(+)23, H8 | 36 | 63 | 80420 +/− 58298 |
| C9 | YKL216W | URA1 | GFP(+)24, E8 | 35 | 62 | 30111 +/− 14692 |
| D9 | YDR447C | RPS17B | GFP(+)29, F1 | 16 | 43 | 83909 +/− 15758 |
| E9 | YJL136C | RPS21B | GFP(+)29, G1 | 10 | 37 | 99708 +/− 53478 |
| F9 | YDR226W | ADK1 | GFP(+)30, A1 | 24 | 51 | 56107 +/− 23955 |
| G9 | YJL140W | RPB4 | GFP(+)30, A2 | 25 | 52 | 7894 +/− 986 |
| H9 | YHR174W | ENO2 | GFP(+)17, E12 | 47 | 74 | 736617 +/− 596163 |
| A10 | YLR075W | RPL10 | GFP(+)30, B2 | 25 | 52 | 74858 +/− 24916 |
| B10 | YGL037C | PNC1 | GFP(+)30, F5 | 25 | 52 | 15409 +/− 6776 |
| C10 | YKL117W | SBA1 | GFP(+)30, A6 | 24 | 51 | 28577 +/− 11847 |
| D10 | YOL139C | CDC33 | GFP(+)30, D8 | 24 | 51 | 40636 +/− 12693 |
| E10 | YDL130W | RPP1B | GFP(+)30, H11 | 11 | 38 | 119013 +/− 96613 |
| F10 | YLR287C-A | RPS30A | GFP(+)30, A12 | 7 | 34 | 46315 +/− 9980 |
| G10 | YKL056C | TMA19 | GFP(+)33, A1 | 19 | 46 | 90676 +/− 40006 |
| H10 | YDR418W | RPL12B | GFP(+)33, C2 | 18 | 45 | 79137 +/− 37701 |
| A11 | YDR155C | CPR1 | GFP(+)33, A4 | 17 | 44 | 95442 +/− 30670 |
| B11 | YPL037C | EGD1 | GFP(+)33, B7 | 17 | 44 | 46777 +/− 28777 |
| C11 | YGL031C | RPL24A | GFP(+)33, H7 | 18 | 45 | 57123 +/− 34444 |
| D11 | YGR148C | RPL24B | GFP(+)33, A8 | 18 | 45 | 36900 +/− 26916 |
| E11 | YJR104C | SOD1 | GFP(+)33, F8 | 16 | 43 | 76043 +/− 43547 |
| F11 | YDR070C | FMP16 | GFP(+)36, D2 | 11 | 38 | 6853 +/− 5916 |
| G11 | YLR325C | RPL38 | GFP(+)36, B6 | 9 | 36 | 32579 +/− 17098 |
| H11 | YJL138C | TIF2 | GFP(+)20, B1 | 45 | 72 | 92322 +/− 25936 |
| A12 | YFR032C-A | RPL29 | GFP(+)36, F9 | 7 | 34 | 72573 +/− 56245 |
| B12 | YOR298C-A | MBF1 | GFP(+)36, A10 | 16 | 43 | 42645 +/− 26071 |
| C12 | YDL061C | RPS29B | GFP(+)36, B10 | 7 | 34 | 31924 +/− 12802 |
| D12 | YDL133C-A | RPL41B | GFP(+)36, H10 | 3 | 30 | 103128 +/− 16021 |
| E12 | YDL184C | RPL41A | GFP(+)36, A11 | 3 | 30 | 106439 +/− 26618 |
| F12 | YBR127C | VMA2 | GFP(+)40, F1 | 58 | 85 | 37171 +/− 10094 |

Information on the molecular weight (MW) and the estimated Molecules/cell originates from the SGD database (https://www.yeastgenome.org/).

Generation of a proteome-wide antibody library. The same library that was described under Examples 3 was used to perform the experiments described in Example 5.

Discovery of novel POI-specific Nanobodies. Similar to Examples 2 to 4 we used NANEX to selectively capture 94 different GFP-POIs from lysates of engineered Yeast strain expressing the different POIs (Table 3) as GFP-tagged proteins (Huh et al., 2003) on 94 separate trapper-coated magnetic beads. After washing, these separate magnetic beads were incubated with the phage displaying the proteome-wide Nanobody library (1,4·10¹⁴ phages) and washed again. Particular for this invention, the GFP-specific stripper Nanobody was then used to selectively elute the immobilized GFP-POIs in association with POIs-specific Nanobodies displayed on phage. Eluted phage were used to infect exponentially grown E. coli TG1 cells and incubated for 30 min at 37° C. without shaking. Next, LB medium (supplemented with 100 μg/mL ampicillin and 2% wt/vol glucose) was added and the culture grown overnight at 37° C. The following day the culture was centrifuged at 3000 g, the cell pellet was resuspended in LB (supplement with 100 μg/mL ampicillin and 20% glycerol (vol/vol)) and stored at −80° C. as a glycerol stock for later use. Yeast lysate from the reference strain EBY100 (which does not contain any GFP-tagged protein) or lysis buffer (without yeast) was spiked with 20 μg of wild type GFP to be used as the (negative) control. 2 rounds of panning were sufficient to observe significant enrichment for nearly all of the 94 different POIs-specific phage (FIG. 12).

For each target selection of binders in this example, as detailed in Table 3, after 2 rounds of selection by panning using NANEX technology, individual clones were isolated from the enriched sub-library by plating tenfold serial dilutions of the overnight-grown phage-infected E. coli TG1 cells in LB. 10 different POIs which showed different enrichment in R2 were chosen, for each POI 12 individual clones were picked and grown in a 96-well plate containing LB supplemented with 100 μg/mL ampicillin. Plasmids containing the Nanobody encoding genes were purified, sequenced and grouped into sequence families (Material and methods). A representative member each sequence family was selected. Their encoding plasmid was transformed into the E. coli WK6 expression strain to express and purify each Nanobody of interest. The specificity of the target-Nanobodies was confirmed by linking separate Nanobodies on NHS-agarose beads for co-immunoprecipitation assays. The beads that were functionalized with these target-specific Nanobodies were incubated for an hour at 4° C. on a rotating device with the lysate of the engineered Yeast strain (Yeast GFP fusion collection reference, as specified Table 3) expressing the target protein as a GFP-tagged protein. After washing, these beads were resuspended in SDS-PAGE loading dye and analyzed on SDS-PAGE. Separated proteins were transferred to a PVDF membrane and the Western blot was developed with GFP-specific antibody (GFP mouse mAb (GF28R), MA5-15256, ThermoFisher Scientific) to confirm the presence of GFP-tagged target (FIG. 13).

From Example 5, we conclude that NANEX allows to easily select target-specific antibodies against several different targets in parallel in a high throughput scale from a proteome-wide antibody library.

TABLE 4
Yeast protein targets used for selection and characterization
of Nb binders (as described in Example 5).

| | Yeast | | MW | MW GFP- |
|---|---|---|---|---|
| Target | collection | Selected | target | target |
| protein | ref. | Nb sequences | (kDa) | (kDa) |
| HSP104 | GFP(+)05 A2 | CA18504 SEQ ID NO: 40 | 102 | 129 |
| MET6 | GFP(+)07 D4 | CA18505 SEQ ID NO: 41 | 86 | 113 |
| SBA1 | GFP(+)30 A6 | CA18508 SEQ ID NO: 42 | 24 | 51 |
| | | CA18509 SEQ ID NO: 43 | | |
| SOD1 | GFP(+)33 F8 | CA18510 SEQ ID NO: 44 | 16 | 43 |
| ENO1 | GFP(+)17 D12 | CA17938 SEQ ID NO: 45 | 47 | 74 |

### Example 6. NANEX to Select Novel Yeast PGI1-Specific Binders from a Proteome-Wide Antibody Library Using a PGI1-Specific Trapper/Stripper Pair and Endogenous PGI1 from a Crude Yeast Cell Lysate

Examples 1 to 5 use a trapper/stripper pair to immobilize GFP as a target or a particular GFP-tagged target to a matrix and subsequently elute this target in association with antigen-specific binding domains or antibodies and their encoding genotype. In Example 6, we show that trapper/stripper pairs can bind directly to an un-tagged target protein, different than GFP, for its immobilization to a matrix and subsequent elution.

Generation of a proteome-wide antibody library. The same library that was described under Example 3 was used to perform the experiments described in Example 6.

Discovery of novel PGI1-specific Nanobodies. Similar to Examples 3, 4 and 5 we used NANEX to selectively capture the POI from yeast lysate. In particular, PGI1-specific Nanobody CA17455 (SEQ ID NO: 15) was immobilized on magnetic beads, as previously described herein, to be used as a trapper. These CA17455-coated beads were incubated for one hour at 4° C. on a rotating device with an EBY100 lysate, comprising the endogenous PGI1 target protein, or with lysate of an engineered Yeast strain (Yeast GFP fusion collection reference GFP(+)12, H11) expressing PGI1 (Systematic Name YBR196C, Table 1) as a GFP-tagged protein (Huh et al., 2003). After washing, these magnetic beads were incubated with the phage displaying the proteome-wide Nanobody library (1,4·10¹⁴ phages) and washed again. Next, the same PGI1-specific Nanobody (herein in the role of a stripper) was then used to selectively elute the immobilized PGI1 in association with PGI1 specific Nanobodies displayed on phage. Selectively recovered phage were used to infect exponentially grown E. coli TG1 cells and incubated for 30 min at 37° C. without shaking. Next, LB medium (supplemented with 100 μg/ml ampicillin and 2% wt/vol glucose) was added and the culture grown overnight at 37° C. The following day the culture was centrifuged at 3000 g, the cell pellet was resuspended in LB (supplement with 100 μg/mL ampicillin and 20% glycerol (vol/vol)) and stored at −80° C. as a glycerol stock for later use. Yeast lysis buffer (which does not contain any yeast protein) was used as the (negative) control. Two rounds of panning were sufficient to observe significant enrichment for PGI1-specific phage.

Individual clones were isolated from the enriched sub-library by plating tenfold serial dilutions of the overnight-grown phage-infected E. coli TG1 cells in LB. 96 individual clones were picked and grown in a 96-well plate containing LB supplemented with 100 μg/mL ampicillin. Plasmids containing the Nanobody encoding genes were purified, sequenced and grouped into sequence families (Material and methods). A representative member of each sequence family was selected. Six different PGI1 specific Nanobodies, derived from four different sequence families (Nb clone CA17791 corresponding to SEQ ID NO: 46, CA17792-SEQ ID NO:47, CA17793-SEQ ID NO:48, CA17794-SEQ ID NO:49, CA17795-SEQ ID NO:50, and CA17796-SEQ ID NO:51) were selected based on their CDR3 sequences. Their encoding plasmid was transformed into the E. coli WK6 expression strain to express and purify each Nanobody of interest. The specificity of the PGI1-specific Nanobodies was confirmed by linking separate Nanobodies on NHS-agarose beads for co-immunoprecipitation assays. The beads that were functionalized with these PGI1-specific Nanobodies and incubated for an hour at 4° C. on a rotating device with either an EBY100 lysate expressing native (un-tagged) PGI1 or the lysate of the engineered yeast strain (GFP(+)12, H11) expressing PGI1 as a GFP-tagged protein. After washing, these beads were resuspended in SDS-PAGE loading dye and analyzed on SDS-PAGE (FIG. 14A). Separated proteins were also transferred to PVDF membranes and the Western blot was developed with a GFP-specific antibody to confirm the presence of the GFP-tagged PGI1 (FIG. 14B). For each PGI1-specific Nanobody analyzed in this way, co-immunoprecipitation with the EBY100 lysate gives a predominant band observed at 61 kDa, corresponding to the expected molecular weight of PGI1. Moreover, when incubated with the lysate of the yeast strain expressing PGI1 as a GFP-tagged protein the predominant band is observed at 88 kDa, consistent with the molecular weight of GFP-PGI1 (27 kDa+61 kDa=88 kDa). Western blot analysis with anti-GFP antibody confirmed that the 88 kDa band contains the GFP-tag.

From this example we conclude that the NANEX-based selection method allows to easily select target-specific antibodies from proteome-wide antibody libraries using a target specific trapper/stripper pair and a native (un-tagged) target, expressed endogenously and captured directly from a crude cell lysate without any additional purification steps.

Further to this we also found that capturing PGI1 from the yeast lysate followed by elution with the stripper, did not just capture PGI1 from the yeast lysate, but a number of interacting proteins were co-eluted and could be identified as binders of PGI1, as captured herein from its cellular context. In particular, PGI1-specific Nanobody CA17455 (SEQ ID NO: 15) was immobilized on magnetic beads, as described above, as a trapper. These CA17455-coated beads were incubated for one hour at 4° C. on a rotating device with an EBY100 lysate, comprising the endogenous PGI1 target protein, and washed. Next, these coated beads were incubated for 1 h with the same PGI1-specific Nanobody (used herein as a stripper) to elute the target protein (PGI1), which revealed several protein bands on SDS-PAGE (FIG. 10), of which the identity was confirmed by mass-spec analysis to involve several PGI1 interacting proteins (LYS20 UniProt P48570, TDH3 UniProt P00359, PNC1 UniProt P53184) (FIG. 10). This finding supports that this method allows to provide target proteins in a physiological context including potential interacting partners for selecting novel binders from a library.

### Example 7. NANEX to Select Novel Nanobodies Specific for a Membrane Protein as Target from an Antibody Library Using a rVGLUT1 Specific Trapper/Stripper Pair and rVGLUT1 from a Cell Lysate

Examples 1 to 6 used a trapper/stripper pair to select for binders against a soluble protein of interest. Here, we used our method to select from the phage displayed Nanobody repertoire of an immune library raised against a membrane protein, in particular the Rat Vesicular GLUtamate Transporter 1 (rVGLUT1, UniProt entry Q62634). In this example, a rVGLUT1-specific Nanobody CA17875 (SEQ ID NO:52) was immobilized on magnetic beads, as previously described herein, to be used as a trapper for the membrane protein rVGLUT1. This immobilized antigen was then incubated with the Nanobody display library derived from the immunized animal. Particular to this invention, the same Nanobody was used as a trapper and then used as a stripper to selectively elute the immobilized rVGLUT1 in association with target-specific Nanobodies displayed on phage. Phage that were selectively recovered with this rVGLUT1-specific stripper were next amplified to generate a new repertoire enriched in Nanobodies specific for rVGLUT1.

Generation of a rVGLUT1 antibody library. Rat VGLUT1 was purified, and llama immunization was performed as described in Schenck et al., 2017. After immunization, a Phage display library was generated as described (Materials and methods).

Discovery of novel rVGLUT1-specific Nanobodies. The full-length rat VGLUT1 was produced in HEK293T cells as published (Schenck et al., 2017) and cells were lysed in 5 ml of ice-cold lysis buffer (250 mM NaCl, 25 mM HEPES pH 7.5, 10% glycerol, 2% DDM) supplemented with protease inhibitors by incubation on a rotating device for 1 h at 4° C. The lysate was clarified by centrifugation at 20,000 g for 20 min. The supernatant containing the target was collected and incubated for 1 h at 4° C. on a rotating device with 5 μL of magnetic beads that were covalently functionalized with the rVGLUT1 specific Nanobody CA17875 (SEQ ID NO:52) to immobilize rVGLUT1 on these beads. The beads were collected using a magnet and were washed with washing buffer (150 mM NaCl, 20 mM Hepes pH 7.5, 10% glycerol and 0.03% DDM) before they were incubated for 1 h at 4° C. with the phage displaying the rVGLUT1 Nanobody library (1,4·10¹⁴ phages). Next, these beads were washed 9 times with 0.5 ml of washing buffer. Particular for this invention, the same rVGLUT1-specific Nanobody (herein in the role of a stripper) was then used to selectively elute the immobilized rVGLUT1 in association with rVGLUT1-specific Nanobodies displayed on phage. Eluted phage were used to infect exponentially grown E. coli TG1 cells and incubated for 30 min at 37° C. without shaking. Next, LB medium (supplement with 100 μg/mL ampicillin and 2% wt/vol glucose) was added and the culture grown overnight at 37° C. The following day the culture was centrifuged at 3000 g, the cell pellet was resuspended in LB (supplemented with 100 μg/mL ampicillin and 20% glycerol (vol/vol)) and stored at −80° C. as a glycerol stock for later use. Lysis buffer (which does not contain any target protein) was used as the (negative) control.

After 2 rounds of selection by panning using NANEX technology, individual clones were isolated from the enriched sub-library by plating tenfold serial dilutions of the overnight-grown phage-infected TG1 cells in LB. 96 individual clones were picked and grown in a 96-well plate containing LB supplemented with 100 μg/mL ampicillin. Plasmids containing the Nanobody encoding genes were purified, sequenced and grouped into sequence families (Material and methods). A representative member of one sequence family of interest was selected and the encoding plasmid was transformed into the E. coli WK6 expression strain to express and purify it (Nb clone CA18425 corresponding to SEQ ID NO:53).

The specificity of the rVGLUT1-specific Nanobody was verified by linking it on NHS-agarose beads for co-immunoprecipitation assays. The beads that were functionalized with this VGLUT1-specific Nanobody were incubated for one hour at 4° C. on a rotating device with a lysate of HEK293T cells transfected with a rVGLUT1 C-terminally tagged with Venus-YFP, and containing a c-Myc tag (Schenck et al., 2017). After washing, these beads were resuspended in SDS-PAGE loading dye and loaded on SDS-PAGE. Separated proteins were transferred to a PVDF membrane and the Western blot was developed with a c-Myc-specific antibody to confirm the presence of the c-Myc-tagged rVGLUT1 (88 kDa for the rVGLUT1-c-Myc-YFP construct, FIG. 15).

From this example we conclude that the NANEX-based selection method allows to select target-specific antibodies from an antibody library using a specific trapper/stripper pair and a membrane target protein.

### Example 8. NANEX to Select Novel rVGLUT1-Specific Nanobodies from a Synaptic Proteome Antibody Library Using a GFP-Specific Trapper/Stripper Pair and a YFP-Tagged Target Protein

The GFP-specific trapper/stripper pair that was introduced in Example 1 can also be used to capture a Yellow fluorescent protein (YFP) tagged target. As the mutations introduced into GFP to create YFP protein do not change the epitope important for trapper/stripper binding, YFP-tagged proteins can efficiently be purified with this GFP-specific trapper/stripper pair from a complex mixture (for example a cell lysate) and to selectively elute the YFP-tagged protein of interest from a matrix (beads, plates, etc). Here, we phage displayed the Nanobody repertoire of an immune library raised against the synaptic proteome, by immunizing a llama with a mouse brain extract enriched in synaptic vesicles, containing—amongst other membrane proteins—the Vesicular GLUtamate Transporter 1 (VGLUT1). In parallel, a GFP-specific trapper CA15816 (SEQ ID NO:2) that was covalently attached to a solid support was used to immobilize a C-terminal Venus-YFP tagged version of rat VGLUT1. This immobilized antigen (rVGLUT1-YFP) was then incubated with the Nanobody display library derived from the immunized animal. Similar to NANEX and particular to this invention, a GFP-specific stripper CA12760 (SEQ ID NO:1) that competes with the trapper was then used to selectively elute the immobilized rVGLUT1-YFP in association with target-specific Nanobodies displayed on phage.

Generation of a synaptic proteome antibody library. Mouse brain extracts enriched in synaptic vesicles were prepared as previously described (Takamori et al., 2006). A llama was immunized weekly over a period of 6 weeks, and a Phage display library was generated as described (Materials and methods).

Discovery of novel VGLUT1-specific Nanobodies. The full-length rat VGLUT1 was produced in HEK293T cells as C-terminal Venus-YFP tagged protein as published (Schenck et al., 2017). The cells were lysed in 5 ml of ice-cold lysis buffer (250 mM NaCl, 25 mM HEPES pH 7.5, 10% glycerol, 2% DDM) supplemented with protease inhibitors by incubation on a rotating device for 1 h at 4° C. The lysate was clarified by centrifugation at 20,000 g for 20 min. The supernatant containing the target was collected and incubated for 1 h at 4° C. on a rotating device with 5 μl of magnetic beads that were covalently functionalized with the GFP-specific Nanobody trapper CA15816 (SEQ ID NO:2) to immobilize YFP-tagged rVGLUT1 on these beads. The beads were collected using a magnet and were washed with washing buffer (150 mM NaCl, 20 mM Hepes pH 7.5, 10% glycerol and 0.03% DDM) before they were incubated for 1 h at 4° C. with the phage displaying the synaptic proteome Nanobody library (1,4·10¹⁴ phages). Next, these beads were washed 9 times with 0.5 mL of washing buffer. Particular for this example, the GFP-specific stripper Nanobody CA12760 (SEQ NO:1) was then used to selectively elute the immobilized rVGLUT1-YFP in association with rVGLUT1-specific Nanobodies displayed on phage. Even though a mouse brain extract was used, the VGLUT1 from mouse and rat only differ in 1 amino acid in their sequence, and thus cross-reactive binders are expected to be present in the mouse-derived library. Eluted phage were used to infect exponentially grown E. coli TG1 cells and incubated for 30 min at 37° C. without shaking. Next, LB medium (supplement with 100 μg/mL ampicillin and 2% wt/vol glucose) was added and the culture grown overnight at 37° C. The following day the culture was centrifuged at 3000 g, the cell pellet was resuspended in LB (supplemented with 100 μg/mL ampicillin and 20% glycerol (vol/vol)) and stored at −80° C. as a glycerol stock for later use. Lysis buffer (which does not contain any target protein) was spiked with 6 μg of wild type GFP to be used as the (negative) control.

After 2 rounds of selection by panning using NANEX technology, individual clones were isolated from the enriched sub-library by plating tenfold serial dilutions of the overnight-grown phage-infected TG1 cells in LB. 96 individual clones were picked and grown in a 96-well plate containing LB supplemented with 100 μg/mL ampicillin. Plasmids containing the Nanobody encoding genes were purified, sequenced and grouped into sequence families (Material and methods). A representative member of each sequence family was selected, and the encoding plasmid was transformed into the E. coli WK6 expression strain to express and purify five Nanobodies of interest (Nb clone CA18024 corresponding to SEQ ID NO:54, CA18437-SEQ ID NO:55, CA18438-SEQ ID NO:56, CA18439-SEQ ID NO:57 CA18440-SEQ ID NO:58, and CA18441-SEQ ID NO:59). The specificity of the rVGLUT1-specific Nanobodies was verified by linking separate Nanobodies on NHS-agarose beads for co-immunoprecipitation assays. The beads that were functionalized with these rVGLUT1-specific Nanobodies were incubated for an hour at 4° C. on a rotating device with a lysate of HEK293T cells transfected with a rVGLUT1-YFP, also containing a c-Myc tag. After washing, these beads were resuspended in SDS-PAGE loading dye and loaded on SDS-PAGE. Separated proteins were transferred to a PVDF membrane and the Western blot was developed with a c-Myc-specific antibody to confirm the presence of the c-Myc-tagged rVGLUT1-YFP (88 kDa, FIG. 16).

From this example we conclude that the NANEX-based selection method allows to select for a panel of novel antibodies specific for membrane protein targets from a proteome-wide antibody library using a specific GFP-trapper/stripper pair and a YFP-tagged membrane protein as target.

### Example 9. NANEX to Select Novel Target-Specific Nanobodies from an Antibody Library Using a mCherry-Specific Trapper/Stripper Pair and a mCherry-Tagged Target Protein

Examples 1 to 5 and 8 used a GFP-specific trapper/stripper pair to capture GFP, GFP-tagged proteins of interest or GFP variants such as YFP for NANEX based selections. In Example 9 a trapper/stripper pair specific for the mCherry tag (Shaner et al., 2004) was used to capture a soluble protein of interest from a complex mixture and to selectively elute said protein of interest from a matrix (beads, plates, etc). Here, we phage displayed the Nanobody repertoire of an immune library raised against a NANEX-purified, crosslinked, full-length, GFP-tagged human Glucocorticoid receptor (GR) by immunizing a llama. In parallel, an mCherry-specific trapper Nb clone CA16964 (SEQ ID NO: 60) that was covalently attached to a solid support was used to immobilize the mCherry-tagged version of the full-length GR. This immobilized antigen (mCherry-GR) was then incubated with the Nanobody display library derived from the immunized animal. Similar to NANEX, an mCherry-specific stripper Nb clone CA17302 (SEQ ID NO: 61) that competes with the trapper Nb was then used to selectively elute the immobilized mCherry-GR in association with GR-specific Nanobodies displayed on phage. Phage that were selectively eluted with this mCherry-specific stripper were next amplified to generate a new repertoire enriched in Nanobodies specific for GR, allowing to repeat this cycle until Nanobodies specific for GR dominated the population to be characterized as monoclonal GR binders.

Generation of a full-length GR antibody library. A construct encoding the Human Glucocorticoid receptor (NR3C1) tagged with GFP was used to express recombinant GFP-GR in HEK293T cells following transient transfection. 48 hours post transfection, cells were collected and lysed in 10 mM Hepes pH 7.4, 10% glycerol, 20 mM Na₂Molybdate, 50 μg/mL DNase I, 10 UM ZnCl₂, 2.5 mM MgCl₂, 2.5 mM DTT, 0.5% NP40 substitute and 1 Complete EDTA-free protease inhibitor tablet using a Dounce homogenizer. GFP-GR was purified to homogeneity as a soluble protein by using a NANEX purification involving the GFP-specific trapper CA15816 (SEQ ID NO:2) and stripper CA12760 (SEQ ID NO:1) pair. The GFP-GR eluted product was used for immunization of a llama weekly over a period of 6 weeks. After immunization, a Phage display library was generated as described (Materials and methods).

Discovery of novel GR-specific Nanobodies. The full-length gene encoding the human Glucocorticoid receptor (NR3C1) was introduced in a pcDNA3.1 expression vector to generate a mCherry-tagged fusion of the target. This vector was transfected in HEK293T cells using poly-ethyleneimine (PEI) as transfection agent. 48 h after transfection, 50 million cells were harvested, washed with ice cold PBS buffer and lysed using a Dounce homogenizer in 5 mL of lysis buffer (10 mM Hepes pH 7.4, 10% glycerol, 20 mM Na₂ Molybdate, 10 μM ZnCl₂, 2.5 mM MgCl₂, 2.5 mM DTT, 0.5% NP40 substitute) supplemented with 50 μg/mL DNAse I and protease inhibitors. The lysate was clarified by centrifugation at 20,000 g. The supernatant containing the mCherry-tagged GR was collected and incubated for 1 h at 4° C. on a rotating device with 5 μl of magnetic beads that were covalently functionalized with the mCherry-specific trapper with reference CA16964 (SEQ ID NO:60) to immobilize the mCherry-tagged GR on these beads. These beads were collected and washed with 0.5 mL washing buffer (10 mM Hepes pH 7.4, 10% glycerol, 20 mM Na₂Molybdate, 10 UM ZnCl₂, 2.5 mM DTT, 0.05% Tween20) using KingFisher™ Flex Purification System (ThermoScientific) prior to incubation for 1 h at 4° C. with the phage displaying the GFP-tagged full length GR Nanobody library (1,4·10¹⁴ phages). Next, these beads were washed 9 times with 0.5 ml of washing buffer. Particular for this invention, the mCherry-specific stripper Nanobody CA17302 (SEQ ID NO:61) was then used to selectively elute the immobilized mCherry-GR in association with GR-specific Nanobodies displayed on phage. Eluted mCherry-GR target protein containing associated phage were used to infect exponentially grown E. coli TG1 cells and incubated for 30 min at 37° C. without shaking. Next, LB medium (supplement with 100 μg/mL ampicillin and 2% wt/vol glucose) was added and the culture grown overnight at 37° C. The following day the culture was centrifuged at 3000 g, the cell pellet was resuspended in LB (supplemented with 100 μg/mL ampicillin and 20% glycerol (vol/vol)) and stored at −80° C. as a glycerol stock for later use. An un-transfected HEK293T lysate (which does not contain mCherry-tagged protein) was spiked with 10 μg of wild type mCherry (mCherry, E. coli Recombinant Protein, TP790040, OriGene) to be used as the (negative) control.

After three rounds of selection by panning using NANEX technology, individual clones were isolated from the enriched sub-library by plating tenfold serial dilutions of the overnight-grown phage-infected TG1 cells in LB. 96 individual clones were picked and grown in a 96-well plate containing LB supplemented with 100 μg/mL ampicillin. Plasmids containing the Nanobody encoding genes were purified, sequenced and grouped into sequence families (Material and methods). A representative member of each sequence family was selected, and the encoding plasmid was transformed into the E. coli WK6 expression strain to express and purify seven Nanobodies of interest (Nb clone CA18498 corresponding to SEQ ID NO: 62, CA18499-SEQ ID NO: 63, CA18501-SEQ ID NO: 64, CA18502-SEQ ID NO: 65, CA18503-SEQ ID NO: 66, CA18585-SEQ ID NO: 67, CA18586-SEQ ID NO: 68). The specificity of the GR-specific Nanobodies was verified by linking separate Nanobodies on NHS-agarose beads for co-immunoprecipitation assays. The beads that were functionalized with these GR-specific Nanobodies were incubated for one hour at 4° C. on a rotating device with a lysate of HEK293T cells transfected with the mCherry-GR expression vector. After washing, these beads were resuspended in SDS-PAGE loading dye and loaded on SDS-PAGE. Separated proteins were transferred to a PVDF membrane and the Western blot was developed with a GR-specific antibody (GR (G-5) mouse IgG2b mAb, sc-393232, Santa Cruz Biotechnology) to confirm the presence of mCherry-tagged GR (FIG. 17).

From this example we conclude that the NANEX-based selection method allows to easily select target-specific antibodies from an antibody library using a mCherry-specific trapper/stripper pair and a mCherry-tagged target protein.

### Example 10. NANEX to Select Novel GFP-Specific Nanobodies from an Antibody Library Using a GFP-Specific Trapper/Stripper Pair and GFP Protein as the Target in a Plate Format

In Examples 1 to 9 NANEX-based selection is performed using magnetic beads (Dynabeads® MyOne™ Tosylactivated, ThermoFisher) covalently coated with a trapper Nanobody. To proof that the NANEX-based selection method as described herein is broadly applicable for different types of matrices, in this Example we show that a trapper/stripper pair can also be used to immobilize a particular target on a surface of a plastic plate, through non-covalent interaction, and elute this target in association with antigen-specific binding domain of antibodies and their encoding genotype.

Generation of a GFP antibody library. The same library that was described under Example 1 was also used to perform the experiments described in Example 10.

Discovery of novel GFP-specific Nanobodies. For the discovery of novel GFP-specific Nanobodies in a plate format, as compared to magnetic beads, we immobilized a biotinylated GFP-specific trapper reference CA15816 (SEQ ID NO:2) on a Neutravidin coated flat-bottom 96-well plate (Nunc Immuno Plate F96 MaxiSorp, 439454, ThermoFisher). These trapper-coated wells were blocked with 4% milk in PBS and washed 5 times with PBS prior to adding 100 μl of 100 nM of GFP (SEQ ID NO:25). Trapper-coated wells that were not incubated with GFP were used as a negative control. Upon incubation with GFP (the antigen), all wells were routinely washed 5 times with PBS and incubated with the GFP antibody library displayed on phage. After an incubation period of 1 h and 30 min at room temperature, the plates were routinely washed 15 times with PBS-Tween₂₀ 0.05%. For the selection of novel GFP-specific antibodies, the non-covalent interaction between the trapper and GFP was selectively disrupted by adding a high-affinity stripper CA12760 (SEQ ID NO:1) that competitively binds to an epitope that overlaps with the trapper epitope. Therefore, the wells were incubated for 30 min with the high-affinity CA12760 (GFP-specific stripper) that binds the same epitope as the trapper on GFP.

For the 2nd round of selection using NANEX, we followed the same strategy, using the output phage obtained from the first round. Four different Nanobody families were identified based on their CDR3 sequences. Among them the three biggest families were identical to three of the families, discovered and confirmed for GFP binding with Bio-layer interferometry (BLI) in Example 1 (CA17518-SEQ ID NO:27, CA17520-SEQ ID NO:29, CA17674-SEQ ID NO:31).

From this example we conclude that the NANEX-based selection method allows to easily select target specific antibodies from antibody libraries using different types of matrices coated with a target-specific trapper Nanobody.

### Materials and Methods

### Cells.

EBY100(ATCC® MYA-4941™)
Genotype: MATa AGA1::GAL1AGA1::URA3 ura352 trp1 leu2delta200 his3delta200 pep4::HIS3 prbd1.6 R can1 GAL.
Yeast GFP Fusion Collection, ThermoFisher, Catalog number: 95702, (Huh et al., 2003).
E. coli TG1 (electrocompetent cells; Lucigen, cat. no. 60502-1) for cloning and preparation of phage libraries.
E. coli WK6 non-suppressor strain (su-) for the expression of Nanobodies (Zell et al., 1987).
HEK 293T (ATCC′ 293T CRL-3216™)

### Monoclonal Antibodies for Western Blotting.

Anti-GFP: mouse mAb (GF28R), MA5-15256, ThermoFisher Scientific.
Anti-GR: (G-5) mouse IgG2b mAb, sc-393232, Santa Cruz Biotechnology.
Anti-c-Myc: mouse mAb (clone 9E10), 11667203001, Roche.

Nanobody coated magnetic beads for panning. NANEX-based selection was performed using magnetic beads (Dynabeads® MyOne™ Tosylactivated, ThermoFisher) coated with a trapper Nanobody specific for GFP (CA15816). 2 mg of purified CA15816 were coated on 50 mg of magnetic beads (˜40 μg antibody/mg beads) according to the manufacturer protocol, beads were then resuspended in 1 ml of PBS.

Yeast lysate for NANEX-based selection. Each selected Yeast clone was grown in 200 ml of YPB media for 72 h at 30° C. (shaking at 175 rpm). Cells were collected by centrifugation for 5 minutes at 4000 rpm. The cell pellet was weighted to normalize all the different lysates. Cell lysates were prepared using the yeast lysis reagent Yper (Y-PER™ Plus, Yeast Protein Extraction Reagent, ThermoFisher), 1 g of cell pellet was resuspended in 2.5 mL Yper (supplemented with 1 mM DTT and EDTA-free protease inhibitor) and incubated for 1 hour at 37° C. Cell lysates were centrifuged for 10 minutes at 20,000 g, the soluble fraction was collected and stored at −80° C.

Negative control. Yeast EBY100 was grown in 200 ml of YPB media for 72 h at 30° C. (shaking at 175 rpm). Cells were collected by centrifugation for 5 minutes at 4000 rpm. The cell lysate was prepared using the yeast lysis reagent Yper (Y-PER™ Plus, Yeast Protein Extraction Reagent, ThermoFisher), 1 g of cell pellet was resuspended in 2.5 mL Yper (supplemented with 1 mM DTT and EDTA-free protease inhibitor) and incubated for 1 hour at 37° C. The cell lysate was centrifuged for 10 minutes at 20,000 g, the soluble fraction was collected and stored at −80° C.

Generation of antibody libraries. The antigen, protein of interest, or proteome of interest was used to immunized a llama. After immunization, a blood sample was collected to clone a diverse set of the affinity matured Nanobodies with specificity for the target or protein of interest. Peripheral blood lymphocytes (PBLs) were isolated from the noncoagulated blood for the purification of total RNA and the synthesis of cDNA. This cDNA served as a template to amplify the open reading frames coding for the variable domains (Nbs) of the heavy-chain antibodies and Nb fragments were cloned into an appropriate phage display vector and phage particles were prepared according to Pardon et al., 2014.

Selection procedure GFP-nanobodies. Pannings or selections for novel GFP Nanobodies was mainly performed as described (Pardon et al., 2014) with an adaptation of the capturing step and the elution step. Briefly, GFP trapper coated beads were used and incubated with different concentration of GFP ranging from 0.1 nM to 100 nM. in a total volume of 100 μL to trap GFP on these NANEX beads after which all magnetic beads were routinely washed 3 times. Next these beads were incubated in 96 well plates with the GFP antibody library displayed on phage. After an incubation period of 2 h at 4° C., beads were routinely washed 12 times with PBS-Tween. For eluting the GFP specific phage, beads were incubated in a total volume of 100 μL for 30 min with 20 UM of the high-affinity CA12760 (GFP-stripper) that binds the same epitope on GFP as the trapper or phage were aspecific eluted from the beads with trypsin (250 μg/mL) for 30 min.

Parallel selection procedure using KingFisher™ Flex Purification System (ThermoScientific). For panning experiments, 300 μL of CA15816-coated magnetic beads were resuspended in 2200 μl of PBS/4% Skimmed Milk and blocked overnight at 4° C. on a rotating device. Before the panning, beads were washed twice in PBS using a magnet. Beads were then resuspended in 480 μL of PBS.

The 12 different Yeast lysates were thawed and 400 μl of each lysate added to a well of a 96 deep-well plate. As negative control for each panning, 400 μl of lysate of the EBY100 strain was supplemented with 20 μg of purified GFP. 20 μL of pre-blocked CA15816-coated magnetic beads were added to each well. The beads and lysates were incubated for 1 h 30 min at 4° C. on a shaking platform.

The next steps of the selection were performed with a KingFisher™ Flex Purification System using 96 well plates. The magnetic beads from each well were harvested and washed for 30 s with 500 μL PBS-Tween. Next these beads were incubated with the proteome-wide antibody library displayed on phage for a period of 1h 30, followed by 9 washes of 30 s with 500 μL PBS-Tween. For eluting the GFP-tagged antigen specific phage, beads were incubated in a total volume of 100 μl for 30 min with 20 M of the high-affinity CA12760 (GFP-stripper) that binds the same epitope on GFP as the trapper.

Identification of selected Nanobody binders. Plasmids containing the Nanobody encoding genes were purified, and then sequenced using MP57 as the sequencing primer. Nanobodies with a similar CDR3 sequence (identical length and >80% sequence identity) were grouped into sequence families (Pardon et al., 2014). It is well known that Nanobodies from the same sequence family derive from the same B-cell lineage and bind to the same epitope on the target.

Nanobody coated agarose beads for antigen co-immunoprecipitation. Antigen-specific Nanobody beads were prepared using NHS-Activated Sepharose 4 Fast Flow (Cytiva) and purified Nanobody. Coupling on the beads was performed according to the manufacturer protocol. 0.5 mg of purified Nanobody were coupled to 150 μl of beads, then resuspended in 0.5 mL of PBS.

Biotinylation. GFP (SEQ ID NO:25) and GFP-specific Nb CA15816 (SEQ ID NO:2) were biotinylated for BLI or NANEX selection, respectively, by using the Thermo Scientific EZ-Link sulfo-NHS-LC-biotinylation kit, according to the manufacturer's instructions.

Epitope mapping of GFP Nanobodies by Bio-layer interferometry (BLI). Streptavidin-coated Octet® biosensors were used to capture biotinylated GFP (100 nM). Unbound biotinylated GFP is washed off from biosensor by two washing steps (60 seconds in buffer). Next, Streptavidin-coated Octet® biosensors with GFP were first incubated for 400 seconds with 20 nM CA12760, the GFP stripper, washed briefly, followed by an incubation of 400 seconds in a premix of 20 nM CA12760 with the different Nbs to be tested. Data were analyzed on an OctetRed (molecular devices). All assays were performed in HEPES 25 mM pH 7.5, NaCl 150 mM supplemented with BSA 0.1% and Tween₂₀ 0.005% at room temperature.

### SEQUENCE LISTING

Amino acid sequences as presented in SEQ ID NO:1-68 (except SEQ ID NO:25) provide for the binders as named therein, and were used in the examples including the C-terminal 6×His tag (also referred to herein as His tag) and EPEA tag, as shown. Further embodiments also include the use of said amino acid binding molecules which have no tags (e.g. SEQ ID NO:70 and SEQ ID NO:71) presenting the VHH sequence of SEQ ID NO:1 and SEQ ID NO:2, resp., or with alternative tags instead of or in addition to said 6×His and EPEA tag, attached to the remaining part of the amino acid sequence at the N-terminus or C-terminus.
SEQ ID NO:1: CA12760 GFP-stripper amino acid sequence (including C-terminal 6×His+EPEA tag)
SEQ ID NO:2: CA15816 T54A/V55A mutant GFP-trapper amino acid sequence (mutated residue in bold underlined; C-term. 6×His+EPEA)
QVQLVESGGGLVQAGGSLRLSCAASGRTFSTAAMGWFRQAPGKERDFVAGIYWAAGSTYYADSAKGRFTISRDNA
KNTVYLQMDSLKPEDTAVYYCAARRRGFTLAPTRANEYDYWGQGTQVTVSSHHHHHHEPEA
SEQ ID NO:3: CA17440 FBA1 binder amino acid sequence (with His-tag and EPEA-tag)
SEQ ID NO:4: CA17441 FBA1 binder amino acid sequence (with His-tag and EPEA-tag)
SEQ ID NO:5: CA17442 FBA1 binder amino acid sequence (with His-tag and EPEA-tag)
SEQ ID NO:6: CA17443 FBA1 binder amino acid sequence (with His-tag and EPEA-tag)
SEQ ID NO:7: CA17451 PDC1 binder amino acid sequence (with His-tag and EPEA-tag)
SEQ ID NO:8: CA17452 PDC1 binder amino acid sequence (with His-tag and EPEA-tag)
SEQ ID NO:9: CA17444 SIS1 binder amino acid sequence (with His-tag and EPEA-tag)
SEQ ID NO:10: CA17453 ALD6 binder amino acid sequence (with His-tag and EPEA-tag)
SEQ ID NO:11: CA17454 ALD6 binder amino acid sequence (with His-tag and EPEA-tag)
SEQ ID NO:12: CA17460 ALD6 binder amino acid sequence (with His-tag and EPEA-tag)
SEQ ID NO:13: CA17458 BMH1 binder amino acid sequence (with His-tag and EPEA-tag)
SEQ ID NO:14: CA17459 BMH1 binder amino acid sequence (with His-tag and EPEA-tag)
SEQ ID NO:15: CA17455 PGI1 binder amino acid sequence (with His-tag and EPEA-tag)
SEQ ID NO:16: CA17456 PGI1 binder amino acid sequence (with His-tag and EPEA-tag)
SEQ ID NO:17: CA17457 PGI1 binder amino acid sequence (with His-tag and EPEA-tag)
SEQ ID NO:18: CA17530 SXM1 binder amino acid sequence (with His-tag and EPEA-tag)
SEQ ID NO:19: CA17560 SSA1 binder amino acid sequence (with His-tag and EPEA-tag)
SEQ ID NO:20: CA17561 SSA1 binder amino acid sequence (with His-tag and EPEA-tag)
SEQ ID NO:21: CA17562 SSA1 binder amino acid sequence (with His-tag and EPEA-tag)
SEQ ID NO:22: CA17563 SSA1 binder amino acid sequence (with His-tag and EPEA-tag)
SEQ ID NO:23: CA17564 SSA1 binder amino acid sequence (with His-tag and EPEA-tag)
SEQ ID NO:24: CA17565 SSA1 binder amino acid sequence (with His-tag and EPEA-tag)
SEQ ID NO:25: GFP protein
SEQ ID NO:26: CA17517 GFP binder amino acid sequence (with His-tag and EPEA-tag)
SEQ ID NO:27: CA17518 GFP binder amino acid sequence (with His-tag and EPEA-tag)
SEQ ID NO:28: CA17519 GFP binder amino acid sequence (with His-tag and EPEA-tag)
SEQ ID NO:29: CA17520 GFP binder amino acid sequence (with His-tag and EPEA-tag)
SEQ ID NO:30: CA17673 GFP binder amino acid sequence (with His-tag and EPEA-tag)
SEQ ID NO:31: CA17674 GFP binder amino acid sequence (with His-tag and EPEA-tag)
SEQ ID NO:32: CA17675 GFP binder amino acid sequence (with His-tag and EPEA-tag)
SEQ ID NO:33: CA17676 GFP binder amino acid sequence (with His-tag and EPEA-tag)
SEQ ID NO:34: CA8780 irrelevant binder amino acid sequence (with His-tag and EPEA-tag)
SEQ ID NO:35: CA17797 GR-LBD binder amino acid sequence (with His-tag and EPEA-tag)
SEQ ID NO:36: CA17798 GR-LBD binder amino acid sequence (with His-tag and EPEA-tag)
SEQ ID NO:37: CA17799 GR-LBD binder amino acid sequence (with His-tag and EPEA-tag)
SEQ ID NO:38: CA17800 GR-LBD binder amino acid sequence (with His-tag and EPEA-tag)
SEQ ID NO:39: CA17801 GR-LBD binder amino acid sequence (with His-tag and EPEA-tag)
SEQ ID NO:40: CA18504 HSP104 binder amino acid sequence (with His-tag and EPEA-tag
SEQ ID NO:41: CA18505 MET6 binder amino acid sequence (with His-tag and EPEA-tag)
SEQ ID NO:42: CA18508 SBA1 binder amino acid sequence (with His-tag and EPEA-tag)
SEQ ID NO:43: CA18509 SBA1 binder amino acid sequence (with His-tag and EPEA-tag)
SEQ ID NO:44: CA18510 SOD1 binder amino acid sequence (with His-tag and EPEA-tag)
SEQ ID NO:45: CA17938 ENO1 binder amino acid sequence (with His-tag and EPEA-tag)
SEQ ID NO:46: CA17791 PGI1 binder amino acid sequence (with His-tag and EPEA-tag)
SEQ ID NO:47: CA17792 PGI1 binder amino acid sequence (with His-tag and EPEA-tag)
SEQ ID NO:48: CA17793 PGI1 binder amino acid sequence (with His-tag and EPEA-tag)
SEQ ID NO:49: CA17794 PGI1 binder amino acid sequence (with His-tag and EPEA-tag)
SEQ ID NO:50: CA17795 PGI1 binder amino acid sequence (with His-tag and EPEA-tag)
SEQ ID NO:51: CA17796 PGI1 binder amino acid sequence (with His-tag and EPEA-tag)
SEQ ID NO:52: CA17875 VGLUT1 binder amino acid sequence (with His-tag and EPEA-tag)
SEQ ID NO:53: CA18425 VGLUT1 binder amino acid sequence (with His-tag and EPEA-tag)
SEQ ID NO:54: CA18024 VGLUT1 binder amino acid sequence (with His-tag and EPEA-tag)
SEQ ID NO:55: CA18437 rVGLUT1 binder amino acid sequence (with His-tag and EPEA-tag)
SEQ ID NO:56: CA18438 rVGLUT1 binder amino acid sequence (with His-tag and EPEA-tag)
SEQ ID NO:57: CA18439 rVGLUT1 binder amino acid sequence (with His-tag and EPEA-tag)
SEQ ID NO:58: CA18440 rVGLUT1 binder amino acid sequence (with His-tag and EPEA-tag)
SEQ ID NO:59: CA18441 rVGLUT1 binder amino acid sequence (with His-tag and EPEA-tag)
SEQ ID NO:60: CA16964 mCherry-trapper binder amino acid sequence (with His-tag and EPEA-tag)
SEQ ID NO:61: CA17302 mCherry-stripper binder amino acid sequence (with His-tag and EPEA-tag)
SEQ ID NO:62: CA18498 GR binder amino acid sequence (with His-tag and EPEA-tag)
SEQ ID NO:63: CA18499 GR binder amino acid sequence (with His-tag and EPEA-tag)
SEQ ID NO:64: CA18501 GR binder amino acid sequence (with His-tag and EPEA-tag)
SEQ ID NO:65: CA18502 GR binder amino acid sequence (with His-tag and EPEA-tag)
SEQ ID NO:66: CA18503 GR binder amino acid sequence (with His-tag and EPEA-tag)
SEQ ID NO:67: CA18585 GR binder amino acid sequence (with His-tag and EPEA-tag)
SEQ ID NO:68: CA18586 GR binder amino acid sequence (with His-tag and EPEA-tag)
SEQ ID NO:69: 6×His-EPEA tag
SEQ ID NO:70: CA12760 GFP-stripper VHH amino acid sequence
SEQ ID NO:71: CA15816 GFP-trapper VHH amino acid sequence

### REFERENCES

Almagro, J. C., et al. (2019). Phage display libraries for antibody therapeutic discovery and development. Antibodies 8: 44.
Huh W K, et al. (2003). Global analysis of protein localization in budding yeast. Nature 425(6959):686-91. Lakzaei et al. (2018). A comparison of three strategies for biopanning of phage-scFv library against diphtheria toxin. J. Cell Physiol. 234 (6), p: 9486-9494.
Li et al. (2020). An unbiased immunization strategy results in the identification of enolase as a potential marker for Nanobody-based detection of Trypanosoma evansi. Vaccines 8, 415.
Lim et al. (2019). Development of a Phage Display Panning Strategy Utilizing Crude Antigens: Isolation of MERS-COV Nucleoprotein human antibodies. Scientific Reports 9, 6088.
Pardon et al. (2014). A general protocol for the generation of Nanobodies for structural biology. Nature Protocols. 9: 674-693.
Schenck et al. (2017) Generation and Characterization of Anti-VGLUT Nanobodies Acting as Inhibitors of Transport. Biochemistry 56(30):3962-3971
Shaner, et al. (2004). Improved monomeric red, orange and yellow fluorescent proteins derived from Discosoma sp. red fluorescent protein. Nat Biotechnol 22, 1567-1572.
Takamori et al. (2006). Molecular anatomy of a trafficking organelle. Cell 127(4):671-3.
Valdorf et al., (2021). Antibody display technologies: selecting the cream of the crop. Biol. Chem. https://doi.org/10.1515/hsz-2020-0377.
Zell et al., (1987). DNA mismatch-repair in Escherichia coli counteracting the hydrolytic deamination of 5-methyl-cytosine residues. EMBO J. 6, 1809-1815

1. A method of isolating a polypeptide binder, the method comprising:
a) mixing a first protein binding agent that is immobilized on a surface and specifically binds a target protein with a first sample comprising the target protein, so as to obtain a complex of the first protein binding agent and the target protein, the complex immobilized on the surface,
b) providing to the complex of step a) a second sample comprising a plurality of polypeptide binders so as to create a mixture,
c) adding to the mixture of step b) a third sample comprising a second protein binding agent, which competes with the first binding agent for specifically binding to the target protein, and wherein specific binding the target protein by the second protein binding agent displaces the first binding agent from the target protein, and
d) eluting the second protein binding agent bound to the target protein, so as to isolate a polypeptide binder bound to the target protein.

2. The method according to claim 1, wherein the rate constant of dissociation (k_{off} value) of the second protein binding agent is lower or equal as compared to the k_{off} value of the first binding agent.

3. The method according to claim 1, wherein the second and/or first protein binding agent comprises an antigen-binding domain.

4. The method according to claim 3, wherein the antigen-binding domain comprises an immunoglobulin single variable domain (ISVD), a VHH, a Nanobody, or an antigen-binding chimeric protein comprising an ISVD fused to a scaffold protein via at least two sites.

5. The method of according to claim 1, wherein the second sample comprising a plurality of polypeptide binders comprises a display library of binding agents.

6. The method according to claim 5, wherein the display library is a recombinant library, and/or an immune library or (semi-)synthetic, non-immune, or naïve library of binding agents, wherein the naïve library of binding agents comprises antibodies, single domain antibodies, ISVDs, VHHs, or nanobodies.

7. The method of according to claim 1, wherein the method is performed using phage, yeast, ribosome, bacteria, or mammalian display and/or wherein after step d), the method steps a) to d) are repeated at least once to enrich the number of polypeptide binders eluting in step d).

8. The method of according to claim 1, wherein the surface comprises (magnetic) beads, a resin, a column, a plate, or a chip.

9. The method of according to claim 1, wherein the first sample comprising the target protein in a) comprises a complex mixture.

10. The method according to claim 9, wherein the complex mixture was applied as an immunogen for obtaining the plurality of polypeptide binders and/or a display library.

11. The method of according to claim 1, wherein the first and/or second protein binding agent specifically binds to a heterologous tag present on the target protein.

12. The method according to claim 10, wherein the tag is GFP or YFP, and/or the first protein binding agent comprises the CDRs of SEQ ID NO:71 and the second protein binding agent comprises the CDRs of SEQ ID NO:70.

13. The method of according to claim 1, wherein the target protein immobilized on the surface is a protein complex comprising at least one or more additional proteins, and/or the polypeptide binder is bound to the complex via binding to at least one or more of the proteins comprised in said protein complex.

14. The method of according to claim 1, wherein step a) and b) are replaced by the following steps:
a. mixing a target protein sample with a sample comprising a plurality of polypeptide binders so as to obtain a mixture, and
b. obtaining an immobilized complex on a surface by adding the mixture of a) to a first protein binding agent, which is preferably immobilized on a surface or subsequently immobilized.

15. (canceled)

16. (canceled)

17. (canceled)

18. The method according to claim 4, wherein the scaffold protein domain comprises HopQ or YgjK.

19. The method according to claim 7, wherein steps a) to d) are repeated at least twice to enrich the number of polypeptide binders eluting in step d).

20. The method according to claim 9, wherein the complex mixture is a biological sample, a cell lysate, or a proteome sample.
